(19) United States
(12) Patent Application Publication
(10) Pub. No.: US 2010/0248390 A1
(43) Pub. Date: Sep. 30, 2010

(54) COMPOSITIONS AND METHODS FOR IDENTIFYING LIGANDS OF ODORANT RECEPTORS

(75) Inventors: Hiroaki Matsunami, Durham, NC (US); Joel D. Mainland, Durham, NC (US)

Correspondence Address: Casimir Jones, S.C., 2275 DEMING WAY, SUITE 310, MIDDLETON, WI, 53562, US

(73) Assignee: DUKE UNIVERSITY, Durham, NC (US)

(21) Appl. No.: 12/715,528

(22) Filed: Mar. 2, 2010

Related U.S. Application Data
(60) Provisional application No. 61/156,703, filed on Mar. 2, 2009.

(51) Int. Cl. G01N 33/566; G06F 19/00

(52) U.S. Cl. 436/501; 702/19

(57) ABSTRACT

The present invention relates to compositions and methods for identifying odorant-odorant receptor interactions. In particular, the present invention relates to in silico methods for identifying odorant receptor-odorant interactions based on chemical and physical properties of odorant receptors and odorants.

### CROSS REFERENCE TO RELATED APPLICATIONS

This application claims priority to provisional application 61/156,703, filed Mar. 2, 2009, which is herein incorporated by reference in its entirety.

### STATEMENT REGARDING FEDERALLY SPONSORED RESEARCH OR DEVELOPMENT

This invention was made with government support under Grant No. DC005782 awarded by the National Institutes of Health. The government has certain rights in the invention.

### FIELD OF THE INVENTION

The present invention relates to compositions and methods for identifying odorant-odorant receptor interactions. In particular, the present invention relates to in silico methods for identifying odorant receptor-odorant interactions based on chemical and physical properties of odorant receptors and odorants.

### BACKGROUND OF THE INVENTION

Olfactory dysfunction arises from a variety of causes and profoundly influences a patient's quality of life. Approximately 2 million Americans experience some type of olfactory dysfunction. Studies show that olfactory dysfunction affects at least 1% of the population under the age of 65 years, and well over 50% of the population older than 65 years. The sense of smell determines the flavor of foods and beverages and serves as an early warning system for the detection of environmental hazards, such as spoiled food, leaking natural gas, smoke, or airborne pollutants. The losses or distortions of smell sensation can adversely influence food preference, food intake and appetite.

Olfactory disorders are classified as follows: 1) anosmia: inability to detect qualitative olfactory sensations (e.g., absence of smell function), 2) partial anosmia: ability to perceive some, but not all, odorants, 3) hyposmia or microsmia: decreased sensitivity to odorants, 4) hyperosmia: abnormally acute smell function, 5) dysosmia (cacosmia or parosmia): distorted or perverted smell perception or odorant stimulation, 6) phantosmia: dysosmic sensation perceived in the absence of an odor stimulus (a.k.a. olfactory hallucination), and 7) olfactory agnosia: inability to recognize an odor sensation.

Olfactory dysfunction is further classified as 1) conductive or transport impairments from obstruction of nasal passages (e.g., chronic nasal inflammation, polyposis, etc.), 2) sensorineural impairments from damage to neuroepithelium (e.g., viral infection, airborne toxins, etc.), 3) central olfactory neural impairment from central nervous system damage (e.g., tumors, masses impacting on olfactory tract, neurodegenerative disorders, etc.). These categories are not mutually exclusive. For example, viruses can cause damage to the olfactory neuroepithelium and they may also be transported into the central nervous system via the olfactory nerve causing damage to the central elements of the olfactory system.

Smelling abilities are initially determined by neurons in the olfactory epithelium, the olfactory sensory neurons (hereinafter “olfactory neurons). In olfactory neurons, odorant receptor (hereinafter “OR”) proteins, members of the G-protein coupled receptor (hereinafter “GPCR”) superfamily, are synthesized in the endoplasmic reticulum, transported, and eventually concentrated at the cell surface membrane of the cilia at the tip of the dendrite. Considering that ORs have roles in target recognition of developing olfactory axons, OR proteins are also present at axon terminals (see, e.g., Mombaerts, P., (1996) Cell 87, 675-686; Wang, F., et al. (1998) Cell 93, 47-60; each herein incorporated by reference in their entireties). In rodents, odorants are transduced by as many as 1000 different ORs encoded by a multigene family (see, e.g., Axel, R. (1995) Sci Am 1273, 154-159; Buck, L., and Axel, R. (1991) Cell 65, 175-187; Firestein, S. (2001) Nature 413, 211-218; Mombaerts, P. (1999) Annu Rev Neurosci 22, 487-509; Young, J. M., et al., (2002) Hum Mol Genet 11, 535-546; Zhang, X., and Firestein, S. (2002) Nat Neurosci 5, 124-133; each herein incorporated by reference in their entirety). Each olfactory neuron expresses only one type of the OR, forming the cellular basis of odorant discrimination by olfactory neurons (see, e.g., Lewcock, J. W., and Reed, R. R. (2004) Proc Natl Acad Sci USA; Malnic, B., et al., (1999) Cell 96, 713-723; Serizawa, S., et al., (2003) Science 302, 2088-2094; each herein incorporated by reference in their entirety).

What is needed is a better understanding of olfactory sensation. What is further needed is a better understanding of odorant receptor function and odorant receptor-odorant interactions.

### SUMMARY OF THE INVENTION

The present invention relates to compositions and methods for identifying odorant-odorant receptor interactions. In particular, the present invention relates to in silico methods for identifying odorant receptor-odorant interactions based on chemical and physical properties of odorant receptors and odorants.

For example, in some embodiments, the present invention provides methods and systems for method of identifying an odorant ligand for an odorant receptor, comprising comparing three or more amino acid property descriptors of an odorant receptor with three or more physicochemical properties of a plurality of odorants; and identifying one or more odorant receptor for the odorant receptor based on the comparing.

In further embodiments, the present invention provides methods and systems for identifying an odorant receptor for an odorant, comprising comparing one or more (e.g., two or more, three or more, five, ten, fifteen, twenty-five, fifty, one hundred, one thousand, etc.) amino acid property descriptors of a plurality of odorant receptors with one or more (e.g., two or more, three or more, five, ten, fifteen, twenty-five, fifty, one hundred, one thousand, etc.) physicochemical properties of an odorant; and identifying one or more odorants receptors for the odorant based on the comparing.

In some embodiments, the physicochemical properties are selected from, for example, Harary H index, topological polar surface area using N, O polar contributions, leverage-weighted autocorrelation of lag 0/weighted by atomic polarizabilities, R autocorrelation of lag 6/weighted by atomic polarizabilities, topological polar surface area using N, O, S, P polar contributions, Radial Distribution Function—11.0/weighted by atomic masses, valence connectivity index chi-2, phenol/enol/carboxyl OH, R autocorrelation of lag 2/weighted by atomic Sanderson electronegativities, R maximal autocorrelation of lag 4/weighted by atomic masses, graph vertex complexity index, Geary autocorrelation—lag 1/weighted by atomic masses, Ha attached to C3(sp3)/C2(sp2)/C3(sp2)/C3(sp), molecular path count of order 04, leverage-weighted autocorrelation of lag 2/unweighted, hydrophilic factor, 2st component symmetry directional WHIM index/weighted by atomic van der Waals volumes, or R maximal index/weighted by atomic polarizabilities. In some embodiments, all of the physiochemical properties are compared. In some embodiments, the amino acid property descriptors are selected from, for example, volume, composition and polarity. In some embodiments, the amino acid property descriptors are compared at specific positions in an alignment of known odorant receptor sequences (e.g., those described in Table 6). In some embodiments, systems and/or methods are performed in silico. In some embodiments, the results of the comparing are displayed on a computer screen. In some embodiments, methods further comprise the step of screening odorants identified in the method using an in vitro assay. In some embodiments, systems are accessed by users within a web-based platform via a web browser across the Internet or as a software package used as a stand-alone system on a single computer.

### DESCRIPTION OF THE FIGURES

FIG. 1 shows EC50 values for 62 odorant receptors and 63 odorants.

FIG. 2 shows bias in odorant and receptor sampling. (A) Odorant space. (B) Receptor space.

FIG. 3 shows that distance in odorant space predicts similarity in receptor response. (A) Testing various odorant-similarity metrics against the functional data. (B) The difference in the receptor response profile is correlated with the distance between the two odorants calculated using 20 optimized descriptors (r=0.79, p<0.001). (C) 18 physicochemical descriptors that explain over 62% of the variance in the dataset. (D) The top ten most similar odorant pairs according to the assay.

FIG. 4 shows that distance in receptor space predicts similarity in responses to odorants. (A) Testing various receptor-similarity metrics against the functional data shows that that the optimized descriptors predict functional responses better than full-sequence similarity or similarity at previously suggested residues. (B) Differences in the odorant response profiles of two receptors are correlated with distances between the same receptors, calculated using 16 optimized descriptors (r=0.73, p<0.001). (C) Snake plot of a typical OR in which amino acid residues with ligand-specificity-determining properties are highlighted.

FIG. 5 shows the breadth of tuning in odorant space. A) Table of assorted breadth of tuning representations. B) A histogram of the hypersphere radius measure for all 62 receptors.

FIG. 6 shows responses of olfactory receptors to enantiomeric pairs.

FIG. 7 shows receptor comparisons by classification. Breadth of tuning did not differ between Class I and Class II receptors by either (A) number of agonists or (B) coverage of odorant space. (C) Sensitivity did not differ between Class I and Class II receptors. Breadth of tuning did not differ between human and mouse receptors by either (D) number of agonists or (E) distance in odorant space. (F) Human receptors were significantly more sensitive to odorants than mouse receptors (p<0.008).

FIG. 8 shows receiver operating characteristic curves for the ligand-receptor interaction classifier. (A) Validation of the model when predicting the response of novel receptors to tested odorants. (B) Validation of the model when predicting the response of tested receptors to novel odorants.

FIG. 9 shows dose response curves for all 340 odorant/receptor interactions.

FIG. 10 shows odorant clustering based on receptor response.

FIG. 11 shows receptor clustering based on response to odorants.

FIG. 12 shows an outline of the screening procedure used in embodiments of the present invention.

FIG. 13 shows a phylogenetic tree of all 464 receptors and 1425 mouse and human odorant receptors.

FIG. 14 shows sensitivity tuning curves.

FIG. 15 shows one dimensional tuning curves.

FIG. 16 shows two-dimensional tuning curves.

FIG. 17 shows a snake plot of a typical OR.

FIG. 18 shows EC50 values for 62 odorant receptors and 63 odorants.

FIG. 19 shows Table 2.

FIG. 20 shows PCA analysis of odorant models of exemplary embodiments of the present invention.

FIG. 21 shows ROC for predicting the response to 6 odorants.

### DEFINITIONS

To facilitate understanding of the invention, a number of terms are defined below.

As used herein, the term “amino acid property descriptors” refers to amino acid properties of odorant receptors that are useful in determining interactions of odorant receptors with odorants. In some embodiments, the descriptors include, but are not limited to, volume, composition and polarity. In some embodiments, the amino acid property descriptors are compared at specific positions in an alignment of known odorant receptor sequences (e.g., those described in Example 1 and Table 6).

As used herein, the term “physiochemical properties” refers to properties of odorants that are useful in determining interactions of odorants with odorant receptors. Examples of physiochemical properties that find use in identifying odorant receptors that are likely to interact with a given odorant include, but are not limited to, Harary H index, topological polar surface area using N, O polar contributions, leverage-weighted autocorrelation of lag 0/weighted by atomic polarizabilities, R autocorrelation of lag 6/weighted by atomic polarizabilities, topological polar surface area using N, O, S, P polar contributions, Radial Distribution Function—11.0/weighted by atomic masses, valence connectivity index chi-2, phenol/enol/carboxyl OH, R autocorrelation of lag 2/weighted by atomic Sanderson electronegativities, R maximal autocorrelation of lag 4/weighted by atomic masses, graph vertex complexity index, Geary autocorrelation—lag 1/weighted by atomic masses, Ha attached to C3(sp3)/C2(sp2)/C3(sp2)/C3(sp), molecular path count of order 04, leverage-weighted autocorrelation of lag 2/unweighted, hydrophilic factor, 2st component symmetry directional WHIM index/weighted by atomic van der Waals volumes, and R maximal index/weighted by atomic polarizabilities.

As used herein, the term “odorant receptor” refers to odorant receptors generated from olfactory sensory neurons.

As used herein, the term “odorant receptor cell surface localization” or equivalent terms refer to the molecular transport of an odorant receptor to a cell surface membrane. Examples of cell surface localization includes, but is not limited to, localization to cilia at the tip of a dendrite, and localization to an axon terminal.

As used herein, the term “odorant receptor functional expression” or equivalent terms, refer to an odorant receptor's ability to interact with an odorant receptor ligand (e.g., an odiferous molecule).

As used herein, the term “olfactory disorder,” “olfactory dysfunction,” “olfactory disease” or similar term refers to a disorder, dysfunction or disease resulting in a diminished olfactory sensation (e.g., smell aberration). Examples of olfactory disorders, dysfunctions and/or diseases include, but are not limited to, head trauma, upper respiratory infections, tumors of the anterior cranial fossa, Kallmann syndrome, Foster Kennedy syndrome, Parkinson's disease, Alzheimer's disease, Huntington chorea, and exposure to toxic chemicals or infections. Diminished olfactory sensation is classified as anosmia—absence of smell sensation; hyposmia—decreased smell sensation; dysosmia—distortion of smell sensation; cacosmia—sensation of a bad or foul smell; and parosmia—sensation of smell in the absence of appropriate stimulus.

As used herein, the terms “subject” and “patient” refer to any animal, such as a mammal like a dog, mouse, rat, pig, cat, bird, livestock, and preferably a human. Specific examples of “subjects” and “patients” include, but are not limited to, individuals with an olfactory disorder, and individuals with olfactory disorder-related characteristics or symptoms.

As used herein, the phrase “symptoms of an olfactory disorder” and “characteristics of an olfactory disorder” include, but are not limited to, a diminished olfactory sensation (e.g., smell aberration).

The phrase “under conditions such that the symptoms are reduced” refers to any degree of qualitative or quantitative reduction in detectable symptoms of olfactory disorders, including but not limited to, a detectable impact on the rate of recovery from disease, or the reduction of at least one symptom of an olfactory disorder.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” “polymorphism,” and “variant” refer to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “competes for binding” is used in reference to a first polypeptide with an activity which binds to the same substrate as does a second polypeptide with an activity, where the second polypeptide is a variant of the first polypeptide or a related or dissimilar polypeptide. The efficiency (e.g., kinetics or thermodynamics) of binding by the first polypeptide may be the same as or greater than or less than the efficiency substrate binding by the second polypeptide. For example, the equilibrium binding constant (K_{D}) for binding to the substrate may be different for the two polypeptides. The term “Km” as used herein refers to the Michaelis-Menton constant for an enzyme and is defined as the concentration of the specific substrate at which a given enzyme yields one-half its maximum velocity in an enzyme catalyzed reaction.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

As used herein, the term “purified” or “to purify” refers to the removal of contaminants from a sample. For example, odorant receptor antibodies are purified by removal of contaminating non-immunoglobulin proteins; they are also purified by the removal of immunoglobulin that does not bind an odorant receptor polypeptide. The removal of non-immunoglobulin proteins and/or the removal of immunoglobulins that do not bind an odorant receptor results in an increase in the percent of odorant receptor-reactive immunoglobulins in the sample. In another example, recombinant odorant receptor polypeptides are expressed in bacterial host cells and the polypeptides are purified by the removal of host cell proteins; the percent of recombinant odorant receptor polypeptides is thereby increased in the sample.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein, is used to indicate a protein that does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “test compound” refers to any chemical entity, pharmaceutical, drug, and the like that can be used to treat or prevent a disease, illness, sickness, or disorder of bodily function, or otherwise alter the physiological or cellular status of a sample (e.g., odorant). Test compounds comprise both known and potential therapeutic compounds. A test compound can be determined to be therapeutic by screening using the screening methods of the present invention.

A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the term “response,” when used in reference to an assay, refers to the generation of a detectable signal (e.g., accumulation of reporter protein, increase in ion concentration, accumulation of a detectable chemical product).

As used herein, the term “reporter gene” refers to a gene encoding a protein that may be assayed. Examples of reporter genes include, but are not limited to, luciferase (See, e.g., deWet et al., Mol. Cell. Biol. 7:725 [1987] and U.S. Pat. Nos., 6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

The term “system” is used to refer to an on-line odorant receptor-odorant interaction system, an example of which is described in the present specification. The term “database” is used to refer to a data structure for storing information for use by the system, and an example of such a data structure in described in the present specification.

The term “user” refers to a person using the systems or methods of the present invention.

As used herein, the terms “processor” and “central processing unit” or “CPU” are used interchangeably and refer to a device that is able to read a program from a computer memory (e.g., ROM or other computer memory) and perform a set of steps according to the program. As used herein, the terms “computer memory” and “computer memory device” refer to any storage media readable by a computer processor. Examples of computer memory include, but are not limited to, RAM, ROM, computer chips, digital video discs (DVD), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc). The term is also intended to encompass non-public networks such as private (e.g., corporate) Intranets.

As used herein, the terms “World Wide Web” or “web” refer generally to both (i) a distributed collection of interlinked, user-viewable hypertext documents (commonly referred to as Web documents or Web pages) that are accessible via the Internet, and (ii) the client and server software components which provide user access to such documents using standardized Internet protocols. Currently, the primary standard protocol for allowing applications to locate and acquire Web documents is HTTP, and the Web pages are encoded using HTML. However, the terms “Web” and “World Wide Web” are intended to encompass future markup languages and transport protocols that may be used in place of (or in addition to) HTML and HTTP. As used herein, the term “web site” refers to a computer system that serves informational content over a network using the standard protocols of the World Wide Web. Typically, a Web site corresponds to a particular Internet domain name and includes the content associated with a particular organization. As used herein, the term is generally intended to encompass both (i) the hardware/software server components that serve the informational content over the network, and (ii) the “back end” hardware/software components, including any non-standard or specialized components, that interact with the server components to perform services for Web site users. As used herein, the term “HTML” refers to HyperText Markup Language that is a standard coding convention and set of codes for attaching presentation and linking attributes to informational content within documents. During a document authoring stage, the HTML codes (referred to as “tags”) are embedded within the informational content of the document. When the Web document (or HTML document) is subsequently transferred from a Web server to a browser, the codes are interpreted by the browser and used to parse and display the document. Additionally, in specifying how the Web browser is to display the document, HTML tags can be used to create links to other Web documents (commonly referred to as “hyperlinks”). As used herein, the term “HTTP” refers to HyperText Transport Protocol that is the standard World Wide Web client-server protocol used for the exchange of information (such as HTML documents, and client requests for such documents) between a browser and a Web server. HTTP includes a number of different types of messages that can be sent from the client to the server to request different types of server actions. For example, a “GET” message, which has the format GET, causes the server to return the document or file located at the specified URL. As used herein, the term “URL” refers to Uniform Resource Locator that is a unique address that fully specifies the location of a file or other resource on the Internet. The general format of a URL is protocol://machine address:port/path/filename. The port specification is optional, and if none is entered by the user, the browser defaults to the standard port for whatever service is specified as the protocol. For example, if HTTP is specified as the protocol, the browser will use the HTTP default port of 80.

As used herein, the term “in electronic communication” refers to electrical devices (e.g., computers, processors, etc.) that are configured to communicate with one another through direct or indirect signaling. For example, a conference bridge that is connected to a processor through a cable or wire, such that information can pass between the conference bridge and the processor, are in electronic communication with one another. Likewise, a computer configured to transmit (e.g., through cables, wires, infrared signals, telephone lines, etc) information to another computer or device, is in electronic communication with the other computer or device. As used herein, the term “transmitting” refers to the movement of information (e.g., data) from one location to another (e.g., from one device to another) using any suitable means.

As used herein, the term “XML” refers to Extensible Markup Language, an application profile that, like HTML, is based on SGML. XML differs from HTML in that: information providers can define new tag and attribute names at will; document structures can be nested to any level of complexity; any XML document can contain an optional description of its grammar for use by applications that need to perform structural validation. XML documents are made up of storage units called entities, which contain either parsed or unparsed data. Parsed data is made up of characters, some of which form character data, and some of which form markup. Markup encodes a description of the document's storage layout and logical structure. XML provides a mechanism to impose constraints on the storage layout and logical structure, to define constraints on the logical structure and to support the use of predefined storage units. A software module called an XML processor is used to read XML documents and provide access to their content and structure.

### DETAILED DESCRIPTION OF THE INVENTION

The present invention relates to compositions and methods for identifying odorant-odorant receptor interactions. In particular, the present invention relates to in silico methods for identifying odorant receptor-odorant interactions based on chemical and physical properties of odorant receptors and odorants.

In experiments conducted during the course of development of embodiments of the present invention, starting with over 450 mouse and human ORs, agonists for 52 mouse and 10 human ORs were identified. These data were used to develop a multidimensional metric for receptor similarity, quantify the breadth of receptor tuning in odorant space, identify ORs capable of discriminating three enantiomeric odorant pairs, distinguish activation profiles of class I and class II ORs, distinguish activation profiles of human and mouse ORs, and develop a model to predict odorant-receptor activation.

Agonists for over five times more mouse ORs than human ORs were identified. The present invention is not limited to a particular mechanism. Indeed, an understanding of the mechanism is not necessary to practice the present invention. Nonetheless, this may indicate, as previously suggested (Menashe et al., BMC Bioinformatics 7, 393 (2006); Young et al., Hum Mol Genet 11, 535-46 (2002); each herein incorporated by reference in their entirety), that mice have even more functional ORs relative to humans than the genome sequences predict. This could reflect a large fraction of nonfunctional genes among the intact human ORs (Mainland, J. Heredity 36, 143-144 (1945); herein incorporated by reference in its entirety) or a large fraction of nonfunctional variants among the human OR clones used for screening. Alternatively, this difference in mouse and human OR response to agonist may be due to bias in odorant choice or technical problems in the heterologous system specific to the human ORs.

Eighteen physicochemical odorant descriptors that predict the functional data were identified. Odorant similarity calculated using these descriptors is highly correlated with similarity using Haddad et al.'s previously identified 32 descriptors (r=0.77, p<0.0001) across a set of over 2500 odorants. The descriptors described herein outperform Haddad et al.'s descriptors (Haddad et al., Nature Methods 5:425 (2008); herein incorporated by reference in its entirety).

Properties of 160R amino acid residues that predict the functional data were identifed.

Of these 16 residues, 12 occur in predicted transmembrane domains, three occur in predicted extracellular domains and one occurs in a predicted intracellular domain. This is consistent with functional evidence and computational predictions that the binding pocket of olfactory receptors is formed by the transmembrane domains (Man et al., Protein Sci 13, 240-54 (2004); Katada et al., J Neurosci 25, 1806-15 (2005); Zhang et al., Genomics 83, 802-11 (2004); Baldwin, Curr Opin Cell Biol 6, 180-90 (1994); each herein incorporated by reference in their entirety). However these residues do not necessarily correspond to the binding site of the ORs, because amino acid residues far from the binding pocket may affect ligand specificity by changing the global conformation of the folded protein.

Enantiomers have very similar physicochemical properties, but mammals can discriminate between members of some enantiomeric pairs (Laska et al., Neuroscience 144, 295-301 (2007); Rubin et al., Nat Neurosci 4, 355-6 (2001); Joshi et al., Chemical Senses 31, 655-64 (2006); Linster et al., J Neurosci 22, 6842-5 (2002); each herein incorporated by reference in their entirety). The (+) and (−) enantiomers of carvone activate overlapping but distinct sets of olfactory neurons in mice (Ma et al., Proc Natl Acad Sci USA 97, 12869-74 (2000); herein incorporated by reference in its entirety). Consistent with these previous reports, it was found that some ORs can distinguish between enantiomers, although many cannot. ORs capable of supplying sufficient information for the discrimination of three enantiomeric pairs were identified. With two exceptions, class I ORs are expressed only in the anterior-dorsal-most zone of the olfactory epithelium in mice (Tsuboi et al, Eur J Neurosci 23, 1436-44 (2006); herein incorporated by reference in its entirety). In agreement with the theory that the olfactory mucosa serves as a chromatographic separator of odorants, with ORs responding to fast-sorbing hydrophilic compounds expressed early in the airstream and ORs responding to slow-sorbing hydrophobic compounds expressed at locations further along in the airstream (Mozell et al., Thorax 46, 21-4 (1991); Schoenfeld et al., Chemical Senses 31, 131-44 (2006); each herein incorporated by reference in their entirety), it was found that class I ORs prefer polar (and therefore hydrophilic) compounds. This indicates that the location of receptor expression along the mucosa may be related to the ligands that bind the receptor.

Humans have approximately 387 potentially functional ORs (in other words, ORs with an intact open reading frame); mice have approximately 1035 (Niimura et al., Gene 346, 23-8 (2005); herein incorporated by reference in its entirety). Despite this difference in OR number, behavioral studies have failed to show a clear distinction between rodents and primates in odorant detection threshold (Laska et al., Chemical Senses 25, 47-53 (2000); herein incorporated by reference in its entirety). The present invention is not limited to a particular mechanism. Indeed, an understanding of the mechanism is not necessary to practice the present invention. Nonetheless, this may be due to a lack of data, or it may be due to compensatory mechanisms such as a shortened nose or more computation at later neural stages (Shepherd, PLoS Biol 2, E146 (2004); each herein incorporated by reference in their entirety). In the present data set it was found, unexpectedly, that human ORs are more sensitive than mouse ORs. Thus, in some embodiments, the present invention provides methods of identifying odorants that are more likely to activate either human or mouse ORs. For example, in some embodiments, the present invention provides a model to predict the interaction of ORs and their ligands.

### I. Olfactory Sensation

The olfactory system represents one of the oldest sensory modalities in the phylogenetic history of mammals. Olfaction is less developed in humans than in other mammals such as rodents. As a chemical sensor, the olfactory system detects food and influences social and sexual behavior. The specialized olfactory epithelial cells characterize the only group of neurons capable of regeneration. Activation occurs when odiferous molecules come in contact with specialized processes known as the olfactory vesicles. Within the nasal cavity, the turbinates or nasal conchae serve to direct the inspired air toward the olfactory epithelium in the upper posterior region. This area (only a few centimeters wide) contains more than 100 million olfactory receptor cells. These specialized epithelial cells give rise to the olfactory vesicles containing kinocilia, which serve as sites of stimulus transduction. There are three specialized neural systems are present within the nasal cavities in humans: 1) the main olfactory system (cranial nerve I), 2) trigeminal somatosensory system (cranial nerve V), 3) the nervus terminalis (cranial nerve 0). CN I mediates odor sensation. It is responsible for determining flavors. CN V mediates somatosensory sensations, including burning, cooling, irritation, and tickling. CN 0 is a ganglionated neural plexus. It spans much of the nasal mucosa before coursing through the cribriform plate to enter the forebrain medial to the olfactory tract. The exact function of the nervus terminalis is unknown in humans. The olfactory neuroepithelium is a pseudostratified columnar epithelium. The specialized olfactory epithelial cells are the only group of neurons capable of regeneration. The olfactory epithelium is situated in the superior aspect of each nostril, including cribriform plate, superior turbinate, superior septum, and sections of the middle turbinate. It harbors sensory receptors of the main olfactory system and some CN V free nerve endings. The olfactory epithelium loses its general homogeneity postnatally, and as early as the first few weeks of life metaplastic islands of respiratory-like epithelium appear. The metaplasia increases in extent throughout life. It is presumed that this process is the result of insults from the environment, such as viruses, bacteria, and toxins.

There are 6 distinct cells types in the olfactory neuroepithelium: 1) bipolar sensory receptor neurons, 2) microvillar cells, 3) supporting cells, 4) globose basal cells, 5) horizontal basal cells, 6) cells lining the Bowman's glands. There are approximately 6,000,000 bipolar neurons in the adult olfactory neuroepithelium. They are thin dendritic cells with rods containing cilia at one end and long central processes at the other end forming olfactory fila. The olfactory receptors are located on the ciliated dendritic ends. The unmyelinated axons coalesce into 40 bundles, termed olfactory fila, which are ensheathed by Schwann-like cells. The fila transverses the cribriform plate to enter the anterior cranial fossa and constitute CN I. Microvillar cells are near the surface of the neuroepithelium, but the exact functions of these cells are unknown. Supporting cells are also at the surface of the epithelium. They join tightly with neurons and microvillar cells. They also project microvilli into the mucus. Their functions include insulating receptor cells from one another, regulating the composition of the mucus, deactivating odorants, and protecting the epithelium from foreign agents. The basal cells are located near the basement membrane, and are the progenitor cells from which the other cell types arise. The Bowman's glands are a major source of mucus within the region of the olfactory epithelium.

The odorant receptors are located on the cilia of the receptor cells. Each receptor cell expresses a single odorant receptor gene. There are approximately 1,000 classes of receptors at present. The olfactory receptors are linked to the stimulatory guanine nucleotide binding protein Golf. When stimulated, it can activate adenylate cyclase to produce the second messenger cAMP, and subsequent events lead to depolarization of the cell membrane and signal propagation. Although each receptor cell only expresses one type of receptor, each cell is electrophysiologically responsive to a wide but circumscribed range of stimuli. This implies that a single receptor accepts a range of molecular entities.

The olfactory bulb is located on top of the cribriform plate at the base of the frontal lobe in the anterior cranial fossa. It receives thousands of primary axons from olfactory receptor neurons. Within the olfactory bulb, these axons synapse with a much smaller number of second order neurons which form the olfactory tract and project to olfactory cortex. The olfactory cortex includes the frontal and temporal lobes, thalamus, and hypothalamus. Although mammalian ORs were identified over 10 years ago, little is known about the selectivity of the different ORs for chemical stimuli, mainly because it has been difficult to express ORs on the cell surface of heterologous cells and assay their ligand-binding specificity (see, e.g., Mombaerts, P. (2004) Nat Rev Neurosci 5, 263-278; herein incorporated by reference in its entirety). The reason is that OR proteins are retained in the ER and subsequently degraded in the proteosome (see, e.g., Lu, M., et al., (2003) Traffic 4, 416-433; McClintock, T. S., (1997) Brain Res Mol Brain Res 48, 270-278; each herein incorporated by reference in their entireties). Despite these difficulties, extensive efforts have matched about 20 ORs with cognate ligands with various degrees of certainty (see, e.g., Bozza, T., et al., (2002) J Neurosci 22, 3033-3043; Gaillard, I., et al., (2002) Eur J Neurosci 15, 409-418; Hatt, H., et al., (1999) Cell Mol Biol 45, 285-291; Kajiya, K., et al., (2001) J Neurosci 21, 6018-6025; Krautwurst, D., et al., (1998) Cell 95, 917-926; Malnic, B., et al., (1999) Cell 96, 713-723; Raming, K., et al., (1993) Nature 361, 353-356; Spehr, M., et al., (2003) Science 299, 2054-2058; Touhara, K., et al., (1999) Proc Natl Acad Sci USA 96, 4040-4045; Zhao, H., et al., (1998) Science 279, 237-242; each herein incorporated by reference in their entirety). Adding the 20 N-terminal amino acids of rhodopsin (e.g., Rho-tag) or a foreign signal peptide to the N-terminus facilitates surface expression of some ORs in heterologous cells (see, e.g., Hatt, H., et al., (1999) Cell Mol Biol 45, 285-291; Krautwurst, D., et al., (1998) Cell 95, 917-926; each herein incorporated in their entirety). However, for most ORs, modifications do not reliably promote cell-surface expression. For example, ODR-4, which is required for proper localization of chemosensory receptors in C. elegans, has a small effect on facilitating cell-surface expression of one rat OR, but not another OR (see, e.g., Gimelbrant, A. A., et al., (2001) J Biol Chem 276, 7285-7290; herein incorporated by reference). These findings indicate that olfactory neurons have a selective molecular machinery that promotes proper targeting of OR proteins to the cell surface, but no components of this machinery have been identified (see, e.g., Gimelbrant, A. A., et al., (2001) J Biol Chem 276, 7285-7290; McClintock, T. S., and Sammeta, N. (2003) Neuroreport 14, 1547-1552; each herein incorporated by reference in their entirety).

For some GPCRs, accessory proteins are required for correct targeting to the cell surface membrane (see, e.g., Brady, A. E., and Limbird, L. E. (2002) Cell Signal 14, 297-309; herein incorporated by reference in its entirety). These proteins include NinaA for Drosophila Rhodopsin (see, e.g., Baker, E. K., et al., (1994) Embo J 13, 4886-4895; Shieh, B. H., et al., (1989) Nature 338, 67-70; each herein incorporated by reference in their entirety), RanBP2 for mammalian cone opsin (see, e.g., Ferreira, P. A., et al., (1996) Nature 383, 637-640; herein incorporated by reference in its entirety), RAMPs for the mammalian calcitonin receptor—like receptor (CRLR) (see, e.g., McLatchie, L. M., et al., (1998) Nature 393, 333-339; herein incorporated by reference in its entirety) and finally the M10 family of MHC class I proteins and beta 2 microglobulin for V2Rs, the putative mammalian pheromone receptors (see, e.g., Loconto, J., et al., (2003) Cell 112, 607-618; herein incorporated by reference in its entirety). With the exception of NinaA and RanBP2, none of these accessory proteins share any sequence homology to with each other; their only common feature is their association with the membrane. The present invention provides novel proteins (e.g., REEP1, RTP1, RTP2, RTP1-A, RTP1-B, RTP1-C, RTP1-D, RTP1-E, RTP1-A1, RTP1-D1, RTP-D2, RTP-D3, RTP1-A1-A (Chimera 1), RTP1-A1-D2 (Chimera 2), RTP1-A1-D1 (Chimera 3), RTP4-A1-A (Chimera 4), RTP4-A1-D2 (Chimera 5), and RTP4-A1-D1 (Chimera 6)) promoting OR cell surface localization and OR functional expression, and numerous compositions and methods related to these findings.

### II. Odorant Receptor-Odorant Interaction Model

In some embodiments, the present invention provides systems and methods for identifying odorant receptor-odorant interactions. In some embodiments, the model provides an in silico method to identify candidate odorants that are likely to interact with a given odorant receptor (e.g., a known or novel odorant receptor). In some embodiments, the present invention provides methods of identifying odorants with binding properties similar to a known odorant. In other embodiments, the model provides an in silico method to identify odorant receptors that are likely to interact with a given odorant.

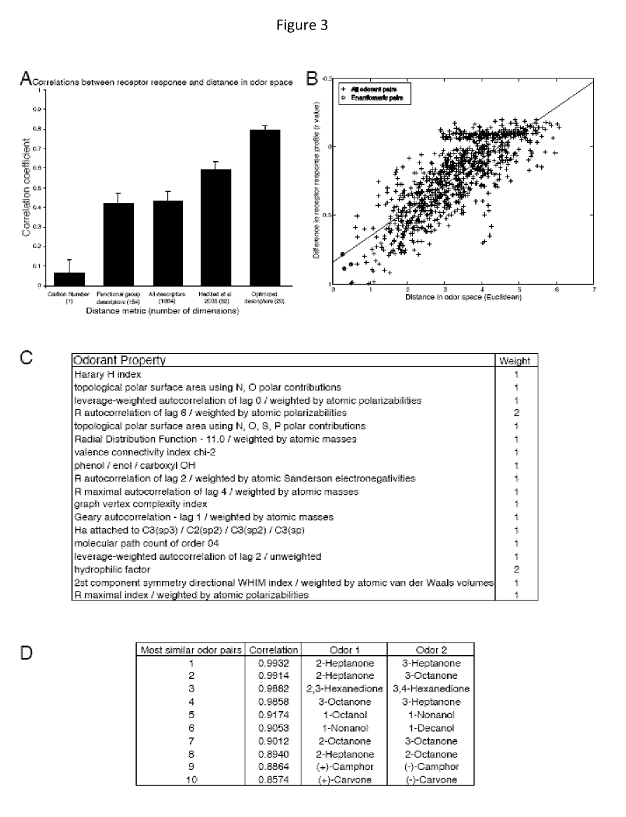

In some embodiments, the model of embodiments of the present invention utilizes one or more (e.g., 2 or more, 5 or more, 10 or more) of the chemicophysical properties of odorants described in FIG. 3 (e.g., Harary H index, topological polar surface area using N, O polar contributions, leverage-weighted autocorrelation of lag 0/weighted by atomic polarizabilities, R autocorrelation of lag 6/weighted by atomic polarizabilities, topological polar surface area using N, O, S, P polar contributions, Radial Distribution Function—11.0/weighted by atomic masses, valence connectivity index chi-2, phenol/enol/carboxyl OH, R autocorrelation of lag 2/weighted by atomic Sanderson electronegativities, R maximal autocorrelation of lag 4/weighted by atomic masses, graph vertex complexity index, Geary autocorrelation—lag 1/weighted by atomic masses, Ha attached to C3(sp3)/C2(sp2)/C3(sp2)/C3(sp), molecular path count of order 04, leverage-weighted autocorrelation of lag 2/unweighted, hydrophilic factor, 2st component symmetry directional WHIM index/weighted by atomic van der Waals volumes, and R maximal index/weighted by atomic polarizabilities). In some embodiments, all of properties described herein are utilized in the model. In some embodiments, additional odorant properties are used in the optimization of the data.

In some embodiments, the model utilizes amino acid property descriptors of odorant receptors (e.g., volume, composition, polarity, relative to the position in an amino acid alignment of known odorant receptor sequences) to model receptor-odorant interactions. In some embodiments, the alignment methods described in Example 1 and the positions described in Table 6 are used in modeling. In some embodiments, additional odorant receptor properties are used in the optimization of the data.

In some embodiments, the present invention provides computer implemented systems and methods for identifying odorant receptor-ordorant interactions. In some embodiments, computer implemented systems and methods generate a report of the results of the modeling methods that provides candidate odorants or odorant receptors. In some embodiments, reports rank a plurality candidate odorants or odorant receptors using a numerical or other scale (e.g., graphical or textual). In some embodiments, the report is provided over the Internet or on a computer monitor.

In some embodiments, the systems and methods of the present invention are provided as an application service provider (ASP) (e.g., can be accessed by users within a web-based platform via a web browser across the Internet; is bundled into a network-type appliance and run within an institution or an intranet; or is provided as a software package and used as a stand-alone system on a single computer).

### III. Uses

The systems and methods described herein find use in a variety of research, clinical, and commercial uses. For example, in some embodiments, the systems and methods are utilized in research applications to indentify candidate odorants or odorant receptors. In some embodiment, the identification of odorant receptor-odorant interactions find use in clinical applications (e.g., in diagnosing or treating diseases of odorant function). In some embodiments, odorants identified using the systems and methods of embodiments of the present invention find use in commercial applications (e.g., as additives in food, cosmetic, perfume, household, or industrial products).

### EXPERIMENTAL

The following examples are provided in order to demonstrate and further illustrate certain preferred embodiments and aspects of the present invention and are not to be construed as limiting the scope thereof.

### Example 1

### A. Materials and Methods

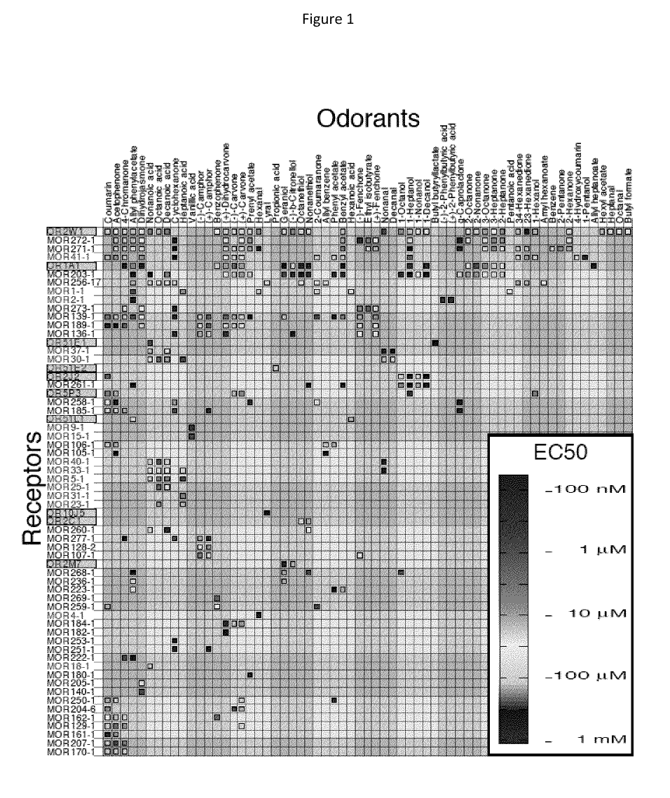

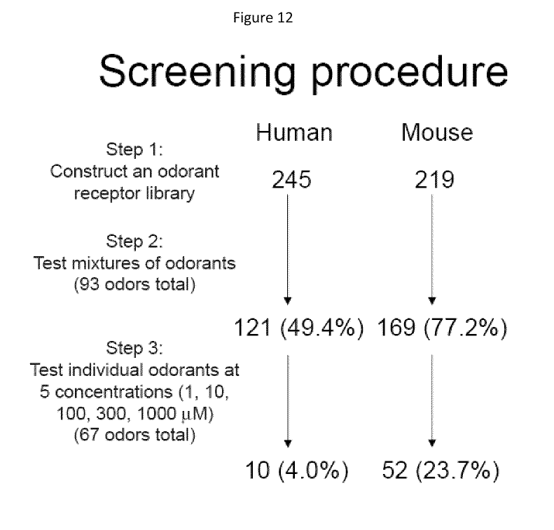

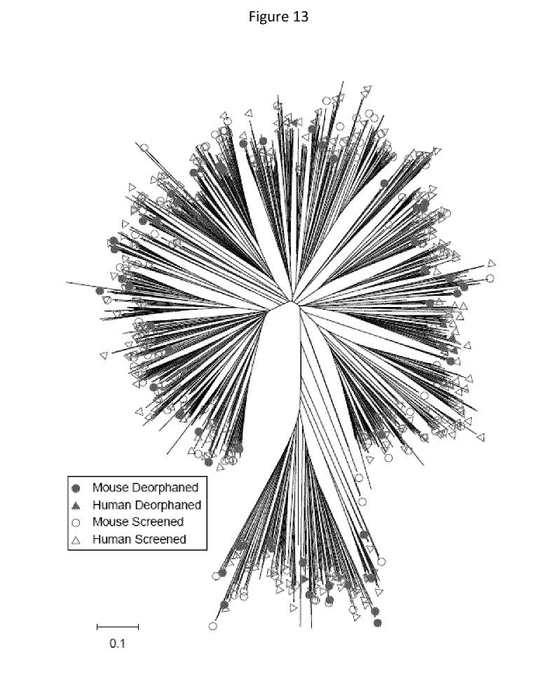

Cloning mouse and human ORs. 219 mouse and 245 human odorant receptors were cloned with sequence information from The Olfactory Receptor Database (Healy et al., Chemical Senses 22, 321-6 (1997); herein incorporated by reference in its entirety). The nomenclature proposed by the D. Lancet group for the human ORs (Glusman et al., Genome Res 11, 685-702 (2001); herein incorporated by reference in its entirety) and by the S. Firestein group for the mouse ORs (Zhang et al., Nat Neurosci 5, 124-33 (2002); herein incorporated by reference in its entirety) was used. OR open reading frames were amplified from genomic DNA using proofreading KOD DNA polymerase (Toyobo/Novagen) and subcloned into pCI expression vectors (Promega) containing the first 20 residues of human rhodopsin (Rho tag). The sequences of the cloned receptors were verified by sequencing (3100 Genetic Analyzer, ABI Biosystems).
Immunocytochemistry and FACS analysis. For live cell-surface staining, the mouse monoclonal anti-rhodopsin antibody, 4D2 (Laird et al., Invest Opthalmol Vis Sci 29, 419-28 (1988); herein incorporated by reference in its entirety) and Cy3-conjugated donkey anti-mouse IgG (Jackson Immunologicals) were used. For FACS analysis, phycoerythrin (PE)-conjugated anti-mouse IgG (Jackson Immunologicals) was used. In order to monitor the transfection efficiency, Green Fluorescent Protein (GFP) was co-transfected with the OR. The intensity of receptor cell surface expression was calculated as the ratio of PE and GFP expression. 7-aminoactinomycin D (Calbiochem) was added before flow cytometry to mark dead cells which were excluded from the analysis. The gate and mean of PE positive cells was normalized by GFP values.
Luciferase assay. Dual-Glo™ Luciferase Assay System (Promega) was used for the luciferase assay as previously described (Saito et al., Cell 119, 679-91 (2004)). Odorant receptor activation leads to an increase in intracellular cAMP; CRE-luciferase (Stratagene) was used to measure this change. Renilla luciferase driven by a constitutively active SV40 promoter (pRL-SV40; Promega) served as an internal control for cell viability and transfection efficiency. Hana3A cells were plated on poly-D-lysine-coated 96-well plates (BioCoat; Becton Dickinson). ORs were transfected with Rho tag in a Hana3A cell line along with RTP1S (Zhuang et al., J Biol Chem 282, 15284-93 (2007); herein incorporated by reference in its entirety), CRE-luciferase, and pRL-SV40 using Lipofectamine2000 (Invitrogen). For each 96-well plate transfected, 1 μg of CRE-Luc, 1 μg of pRL-SV40, 5 μg of odorant receptor plasmid, and 1 μg of RTP1S was used. Approximately 24 hours post transfection, the medium was replaced with CD293 chemically defined medium (Gibco) and the plate was incubed for 30 min at 37° C. The medium was then replaced with 25 μL of odorant solution diluted in CD293 and the plate was incubated for 4 hrs at 37° C. and 5% CO2. The manufacturer's protocols for measuring luciferase and Renilla luciferase activities was followed. Luminescence was measured using a Wallac Victor 1420 plate reader (Perkin-Elmer). All luminescence values were divided by the Renilla Luciferase activity to control for transfection efficiency in a given well. Normalized luciferase activity was calculated using the formula (LN−Lmin)/(Lmax−Lmin), where LN is the luminescence of firefly luciferase in response to the odorant, Lmin is the minimum luciferase value on a plate or set of plates, and Lmax is the maximum luciferase value on a plate or set of plates. The data was analyzed using Microsoft Excel and GraphPad Prism 4.
Screening procedure. The entire OR library was stimulated with eight separate odorant mixtures formed from 93 odorants (FIG. 12). The mixtures were applied at 100 μM and all ORs that did not show activity (the ratio of CRE-Luciferase to Renilla luciferase was less than 0.1 above baseline) were eliminated. The mixtures were applied at five different doses (1 μM, 10 μM, 100 μM, 300 μM and 1 mM) and all ORs that did not show dose-dependent activity to any of the eight mixtures (statistical significance was not assessed for this screening stage) were eliminated, leaving 121 human receptors (49.4%) and 169 mouse receptors (77.2%). A comparison between the 93 individual odorants at a 100 μM dose and a no-odor control was performed for all 290 odorants. Each comparison was performed in triplicate; statistical significance was assessed by t-test (uncorrected for multiple comparisons). In addition, the consistency of the experimental conditions was confirmed with two positive controls: MOR203-1 with nonanoic acid, and MOR32-1 with nonanoic acid. 27 human ORs (11.0%) and 102 mouse ORs (46.6%) showed a significant response to at least one of 67 odorants relative to a no-odor control. Dose-response curves ranging from 10 nM to 3 mM were constructed for each combination of 129 receptors and 67 odorants. A single odorant was used on each plate to avoid cross-contamination, and each OR odorant dose was tested at least three times. The data was fit to a sigmoidal curve. An odorant/receptor pair was counted as a significant activation if both the normalized activity at 100 μM was significantly different than the baseline activity (using a t-test), and the standard deviation of the fitted log EC50 was less than 0.5 log units. It was confirmed that the raw CRE-Luc curve and the normalized (Luc/RL) curve EC50 values did not differ by more than 1 log step. As a result, 52 mouse (23.7%) and 10 human (0.04%) ORs that showed a significant dose-dependent response to one or more of 63 odorants were identified (FIG. 1). Four odorants failed to activate any OR in this final stage.
Physicochemical descriptors. Molecular structure files were obtained for each odorant from PubChem and these structures were inputted into the Virtual Computational Chemistry Laboratory (Tetko et al., J. Comput. Aid. Mol. Des. 19, 453-63 (2005); herein incorporated by reference in its entirety). Then, CORINA (Gasteiger et al., Tetrahedron Comput. Method 3, 537-547 (1992); herein incorporated by reference in its entirety) was used to obtain 3D coordinates and Dragon (Talete) to compute 1664 physicochemical descriptors.
Receptor descriptors. 14250R amino acid sequences from Niimura and Nei (Niimura et al., Gene 346, 23-8 (2005); herein incorporated by reference in its entirety) and 464 OR amino acid sequences from an OR library were aligned using the MUSCLE algorithm (Edgar, BMC Bioinformatics 5, 113 (2004); herein incorporated by reference in its entirety) in Seaview (Galtier et al., Comput Appl Biosci 12, 543-8 (1996); herein incorporated by reference in its entirety) with manual adjustment for conserved domains (Table 2). All sites that were gaps in over 90% of the 1425 ORs were eliminated, leaving 327 amino acids. The set of 981 descriptors consisted of the polarity, composition and volume of these 327 residues, as defined by Grantham (Grantham, Science 185, 862-4 (1974); herein incorporated by reference in its entirety). Differences between disease alleles and wild-type alleles computed using these properties are on average greater than those observed between putatively neutral polymorphic alleles, indicating that these properties are functionally relevant (Miller et al., Hum Mol Genet 10, 2319-28 (2001); herein incorporated by reference in its entirety).
Correlations between descriptors and responses. Each descriptor was z-scored across 2683 odorants (Table 5) or all 1425 receptors (Table 2). All odorants for which there were fewer than 3 responsive receptors were eliminated. For all remaining odorant pairs the Pearson correlation between EC50 vectors and the Euclidean distance between descriptor vectors was calculated. The Pearson correlation between these two sets of distances was then measured.
Optimizing descriptors. A greedy optimization algorithm, as in (Haddad et al., Nature Methods 5, 425-429 (2008); herein incorporated by reference in its entirety), was used to determine the best set. In this method one begins with an empty set of descriptors. Each descriptor is combined with the previous set of descriptors and correlation values are computed for all of these candidate sets of descriptors. To reduce overfitting, the data is randomly divided into ten subsets and correlation values are computed for each leave-one out subset. The final correlation coefficient for each set of descriptors is the average of the correlation coefficients for each subset. The set with the best correlation coefficient then becomes the new set of descriptors and the process is repeated until the correlation coefficient increases by less than 0.004 in three consecutive iterations.

This method was validated on each dataset using a leave-10-out cross-validation scheme. In other words, the descriptors were optimized using 90% of the data, and then tested on the remaining 10% of the data. This division was repeated ten times such that all subdivisions were test sets and the average performance over all test sets was reported. It was then verified that this optimization does not work as well for randomly shuffled vectors. The same descriptor values as in the actual vectors was used, but shuffled each descriptor independently so that any given object had a random set of descriptor values. 30 sets of these objects with shuffled descriptor values were created. For each of these 30 sets descriptors were optimized for 90% of the data and the descriptors were tested on the remaining 10% of the data. The average of all 30 values was reported. The performance of the real optimized descriptors was compared to the shuffled optimized descriptors using a two-sample t-test with unequal variance.

Breadth of tuning. A receptor's breadth of tuning is defined as the radius of a hypersphere, centered on the center of mass of all of the receptor's agonists and enclosing all of the receptor's agonists in Haddad et al's (supra) odorant space. For reference, a hypersphere enclosing 2,683 odorants (Table 6) has a radius of 26; a hypersphere enclosing the 93 odorants in the test set has a radius of 14; a hypersphere enclosing the 63 odorants that activated at least one receptor has a radius of 12.
Machine-learning algorithm. The support vector machines (SVM) functions in the Bioinformatics Toolbox of Matlab (Mathworks) were used to classify the odorants in the data set. To estimate the discrimination ability of the classifier for class I/class II discriminations and human/mouse discriminations, the data set was jackknifed. In other words, the classifier was trained on all but one instance and then tested on that instance. This was repeated n times, where n was the total number of instances. Signal detection theory was used to compute the sensitivity index (d′), which is the separation of the means of the two distributions (class I and class II agonists, or mouse and human agonists) in units of standard deviation. It was confirmed that the d′ was significantly different from zero according to Marascuilo's test (Marascuilo, Psychometrika 35, 237-243 (1970); herein incorporated by reference in its entirety). The Rank features function in the MATLAB Bioinformatics Toolbox to determine the physicochemical properties that best predict membership in a class. A cross-correlation weighting value of 0.7 was applied to reduce the number of highly correlated properties.
Predicting odorant-OR interactions. Each of the 3,906 tested odorant-OR interactions were represented by a vector of 1664 physicochemical descriptors and 981 receptor descriptors. A cross-validation procedure was used to determine if a subset of these vectors could predict if the odorant-OR combination resulted in activation (i.e. all colored squares in FIG. 1). A ten-fold validation procedure was used in two different ways. In the first method, all 62 rows of odorant-OR interactions were divided into 10 sets (leave-receptor-out). In the second method, all 63 columns of odorant interactions were divided into ten sets (leave-odorant-out). After choosing the test set for a round the greedy optimization method described above was used to calculate optimized descriptor sets for the other nine sets (training data). After this selection of attributes logistic regression in JMP v6 (SAS Institute) was performed on the training set and the resulting coefficients used to predict the probability of activation for the test set. This method was repeated ten times for each of the three selection processes, rotating the test set each iteration, such that the entire data set was evaluated using a model that was not trained on its respective test set. Receiver operating characteristic curves were generated according to (Fawcett, Pattern Recognition Letters 27, 861-874 (2006); herein incorporated by reference in its entirety). Statistical significance was determined using a Mann-Whitney U-test comparing the distribution of predicted odds for interactions resulting in activation to the distribution of predicted odds for interactions resulting in no activation.

### B. Results

### High-Throughput Screening of Mouse and Human ORs

Libraries of mouse and human ORs that represent a large fraction of the total mouse and human OR families were generated (Table 2). The mouse OR library comprises 219 mouse ORs that represent over 21% of the total 1035 mouse OR genes and includes at least one member of 217 out of the 228 mouse OR subfamilies defined by Zhang and Firestein (Nat Neurosci 5, 124-33 (2002); herein incorporated by reference in its entirety). The human OR library comprises 245 ORs that represent 63% of the 387 human OR genes. Although many human OR genes are polymorphic (I. Menashe, et al., PLoS Biol 5, e284 (2007); A. Keller, et al., Nature 449, 468-72 (2007); I. Menashe, et al., Nat Genet 34, 143-4 (2003); each herein incorporated by reference in their entirety), a single variant of each receptor was used in the screen (Table 2).

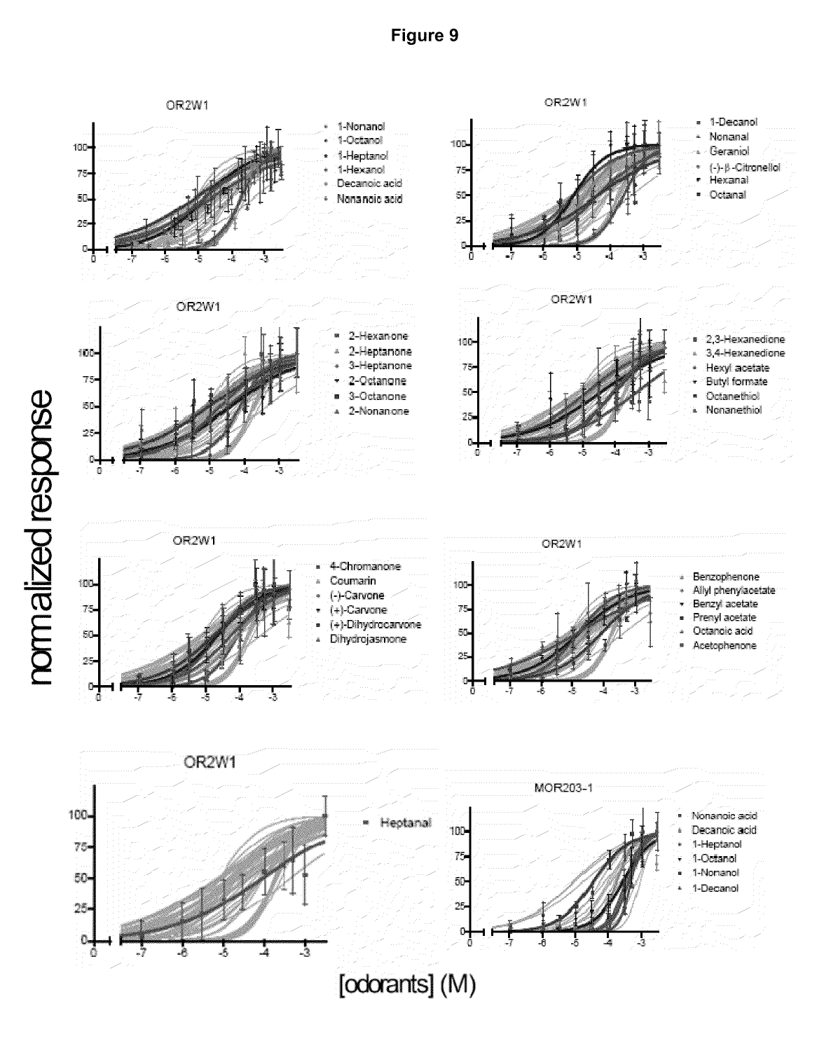

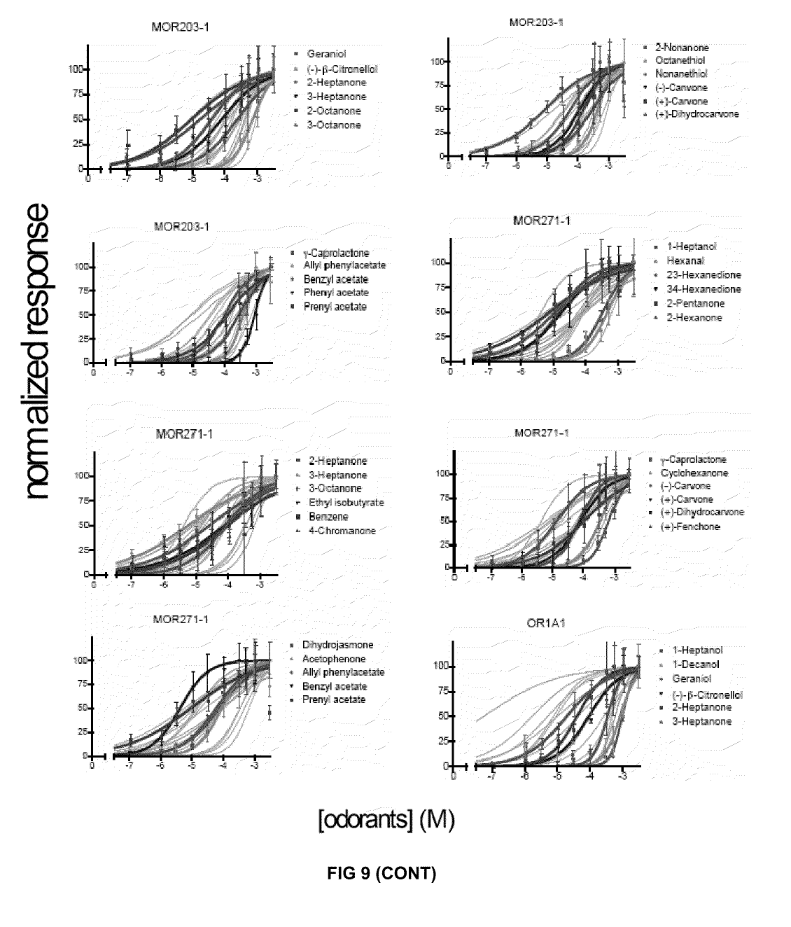

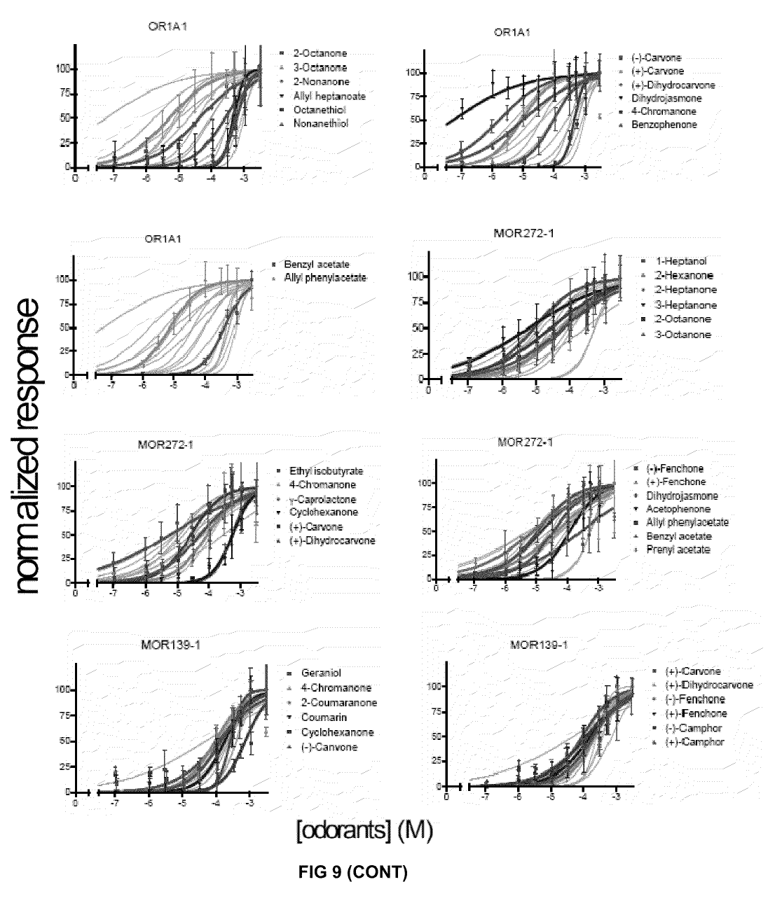

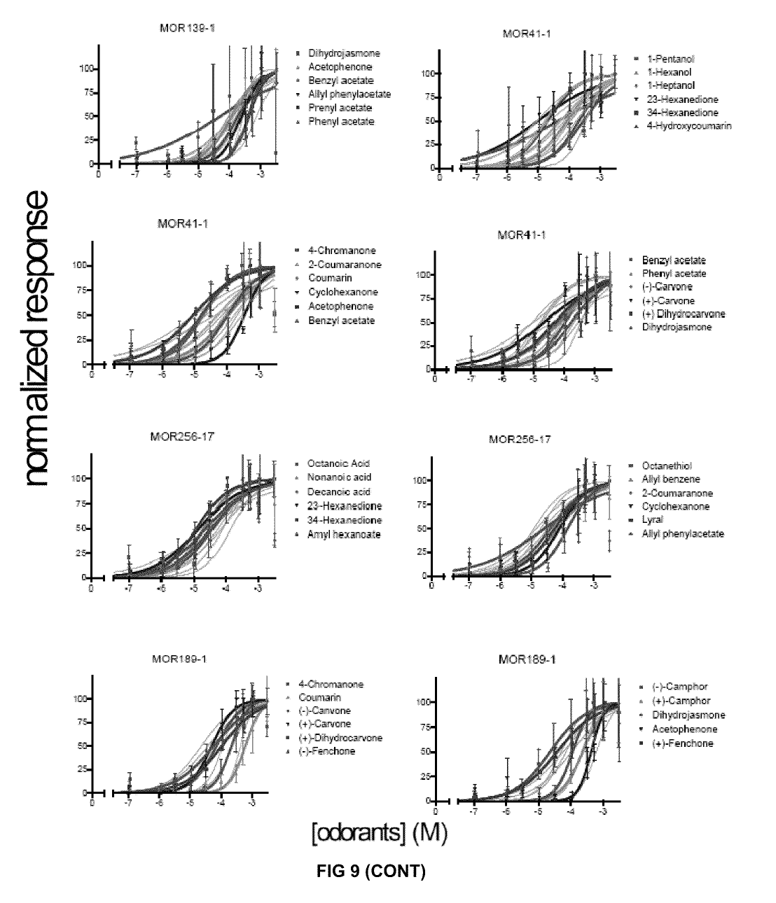

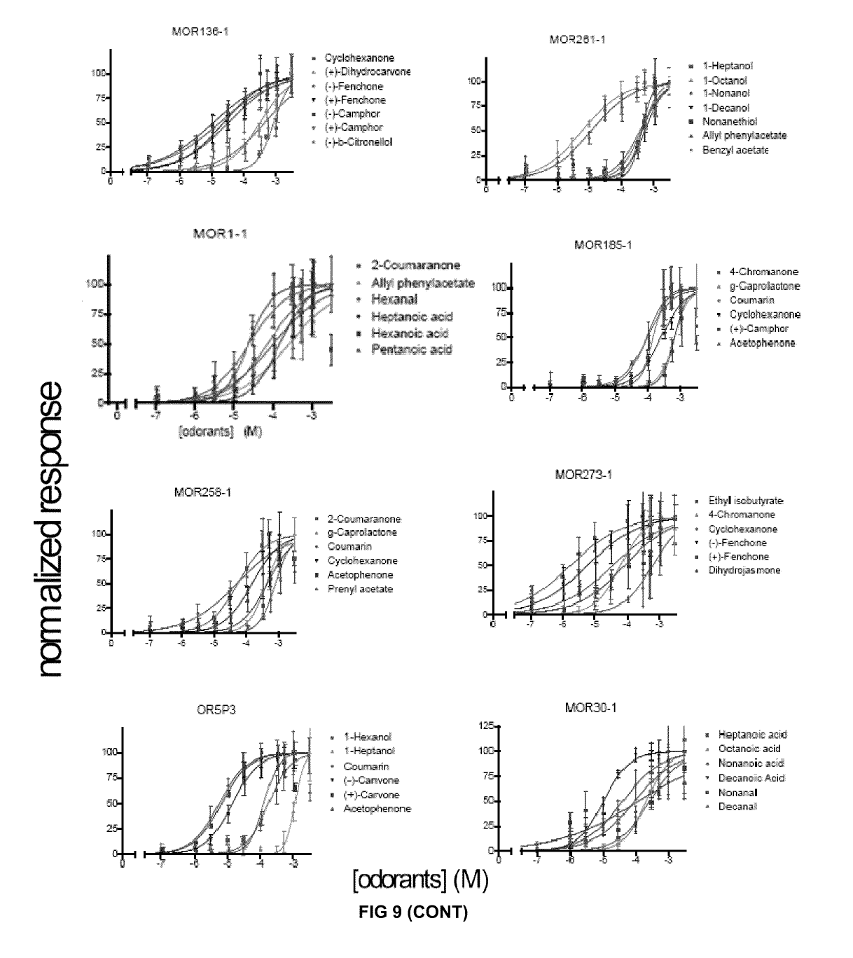

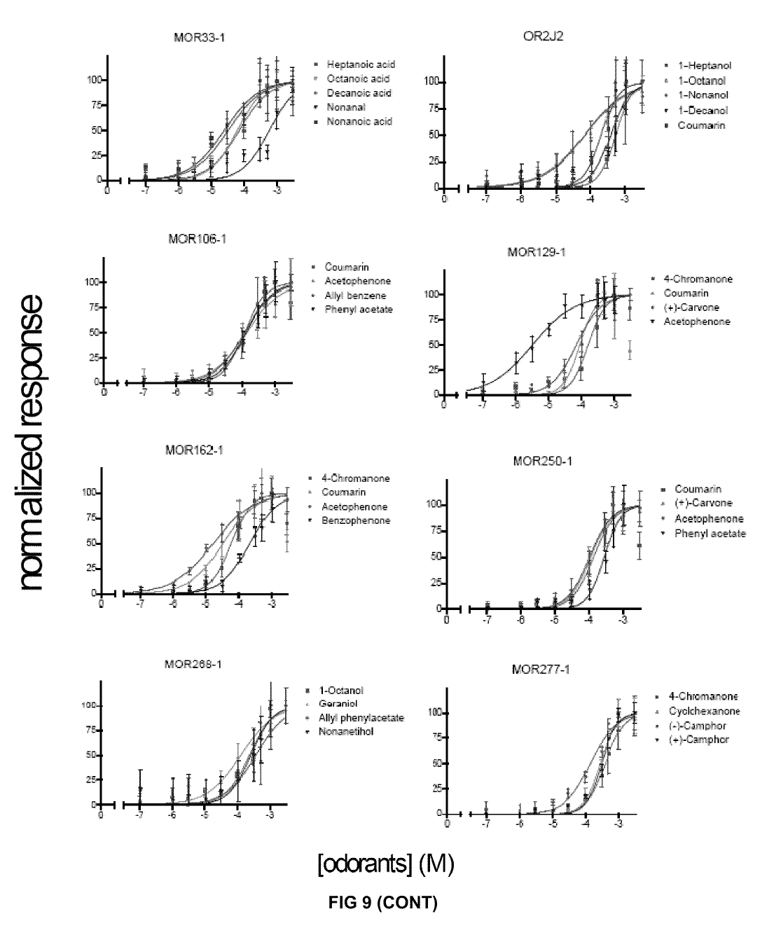

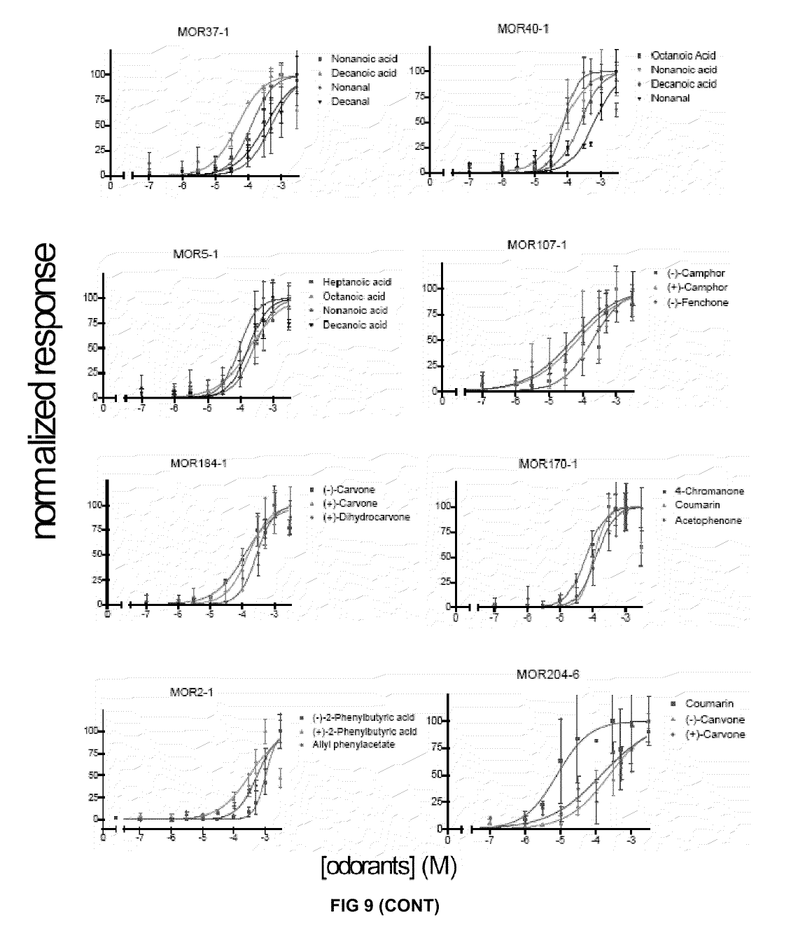

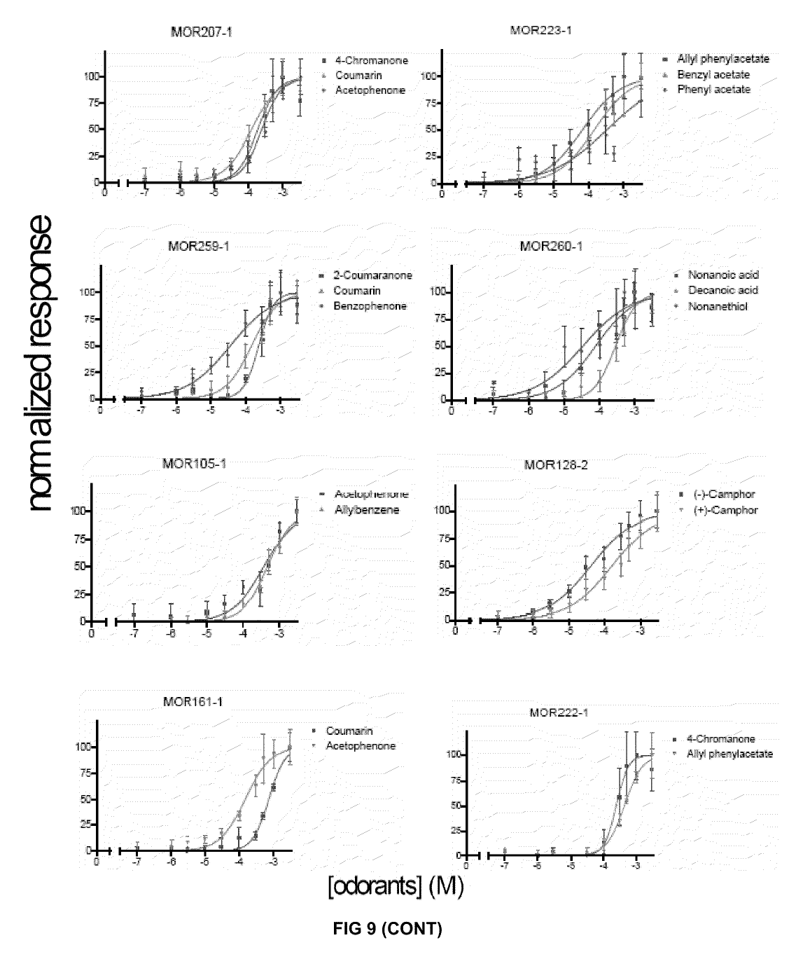

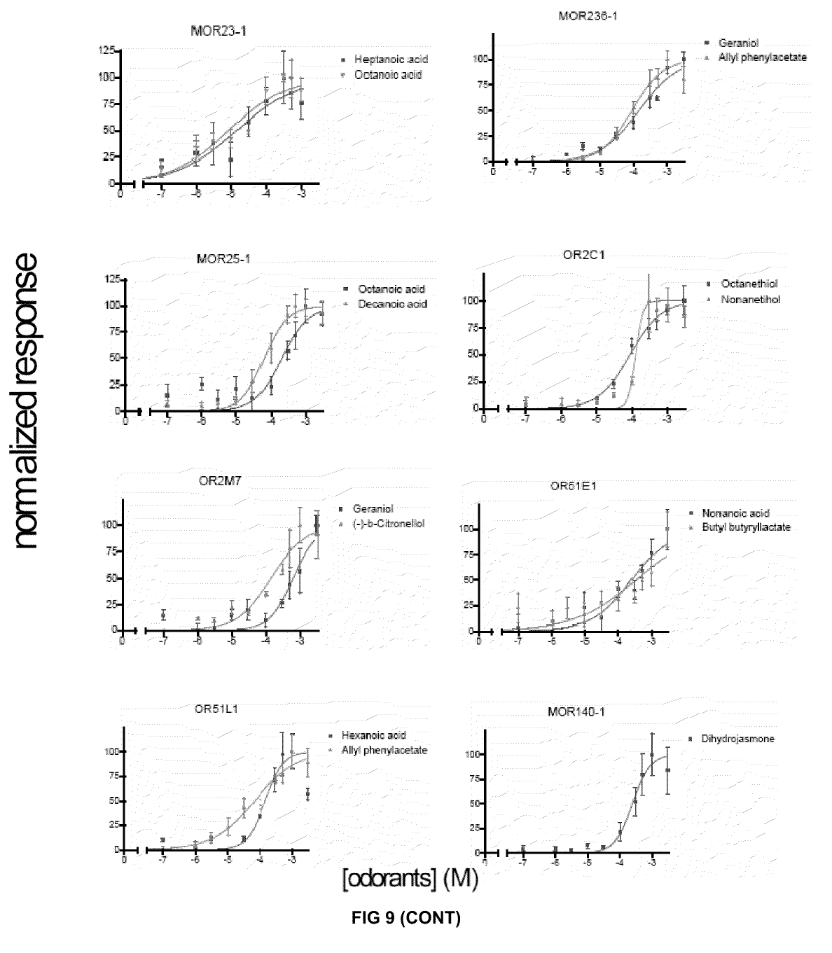

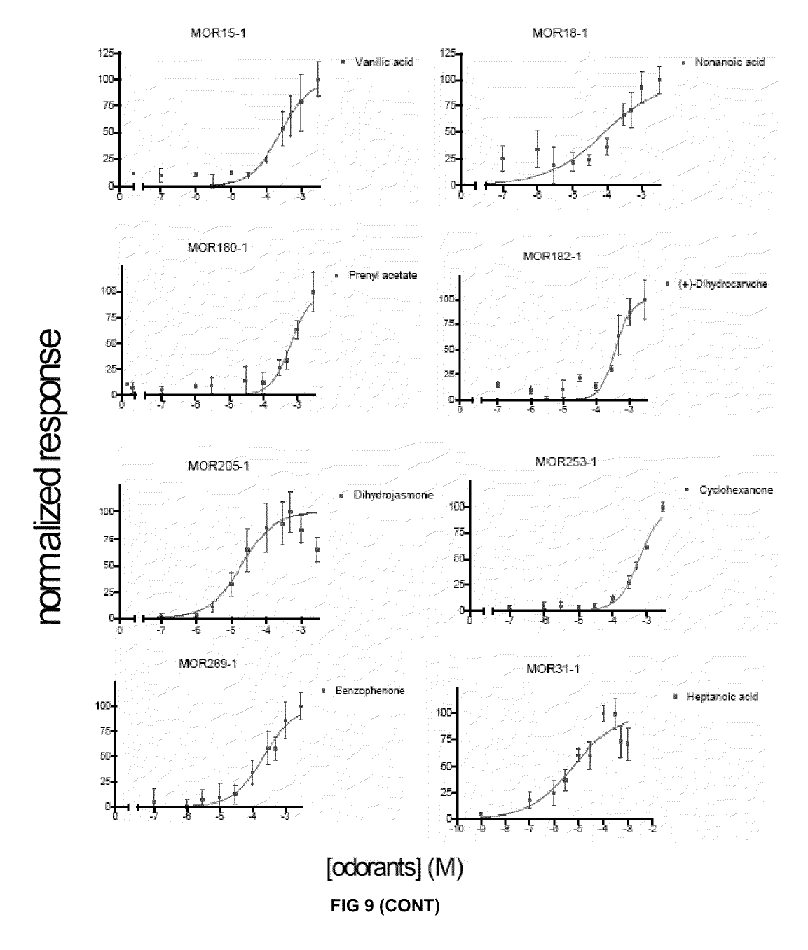

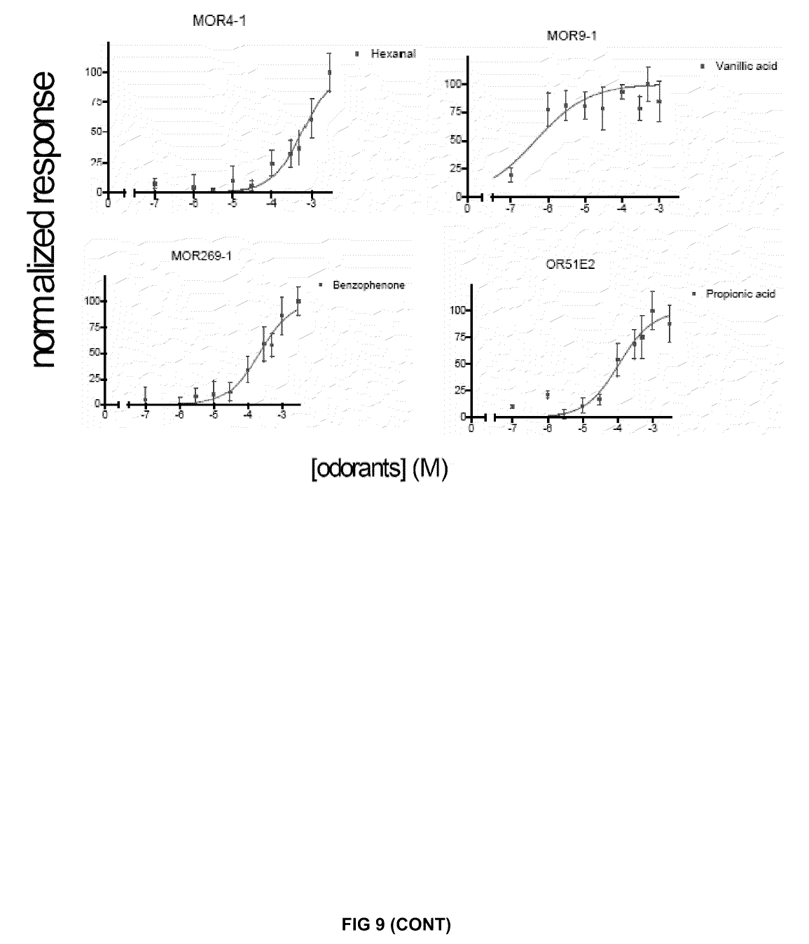

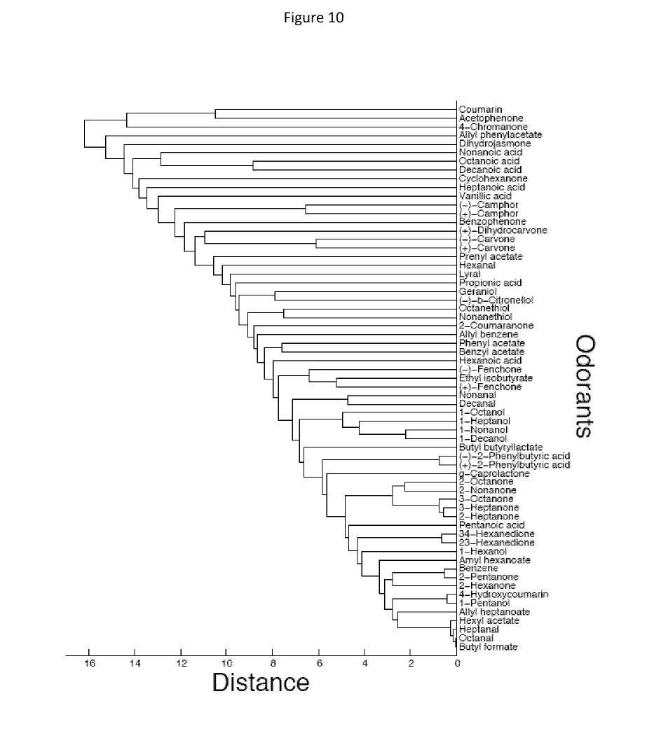

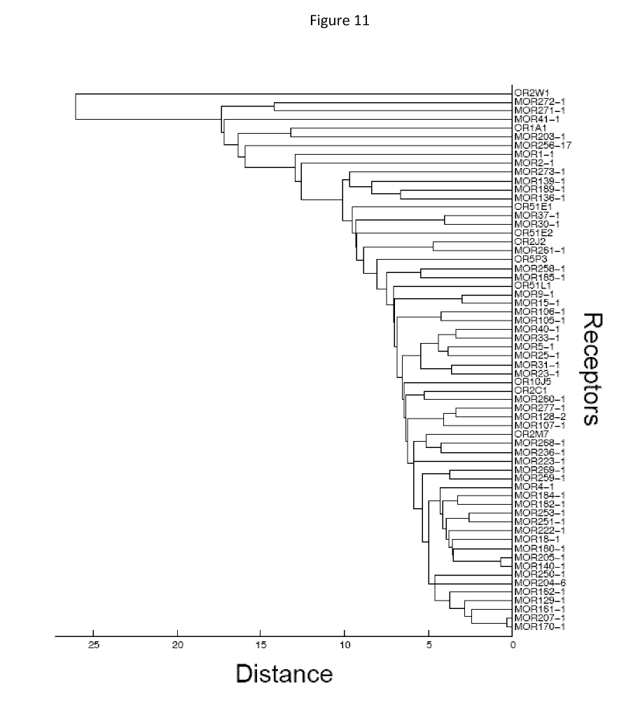

The entire OR library was stimulated with one of 8 odorant mixtures drawn from 93 odorants chosen to represent diverse functional groups, sizes, and structures (Table 3). Each mixture was applied at five different concentrations. 121 human ORs (49.4%) and 169 mouse ORs (77.2%) showed a response to at least one mixture at one concentration. The 93 odorants were then applied individually at 100 μM to the 290 mixture-responsive ORs. 27 human ORs (11.0%) and 102 mouse ORs (46.6%) showed a significant response [p<0.05, uncorrected for multiple comparisons] to at least one of 67 odorants relative to a no-odor control. Dose-response curves were then prepared for every combination of 129 receptors and 67 odorants that showed a response significantly above baseline in the previous step. In this more stringent test, 52 mouse and 10 human ORs were identified that responded to one or more of 63 odorants (FIG. 1, also see FIG. 9-11 and Table 4), representing 23.7% of the original mouse library and 4% of the original human library. The additional four odorants and 67 receptors did not elicit or show a significant response in this follow-up. Although the positive responses resulted in discovery of a large number of OR agonists, the failure of a specific OR to respond to any of the tested odorants may reflect a failure of the OR to function in the assay rather than a lack of sensitivity to the tested odorant. In further analyses on results for receptors that responded to at least one of the tested odorants, and were therefore unequivocally functional in the assay, were analyzed.

### Bias in Odorant and Receptor Representation

One problem in the field of olfaction research is the lack of an agreed-upon metric to organize odorants; for example there is currently no agreed-upon metric to quantify odorant similarity (Haddad et al., Current Opinion in Neurobiology 18, 1-7 (2008); herein incorporated by reference in its entirety). Without such a metric it is difficult to choose a random sample of odorants that fairly represents all odorants, as all odorants have discrete molecular structures that differ in such physicochemical properties as molecular weight, carbon number, functional groups, and hydrophobicity. A bias in odorant sampling may give a misleading estimate of a receptor's preferred physicochemical features as well as lead to faulty extrapolation of odorant similarity. Due to the large number of physicochemical properties thought to determine odorant similarity, a broad descriptor set was used to show the bias in the chosen odorant set and principal component analysis (PCA) was used to simplify the organization of these odorants. PCA is a method for transforming a number of possibly correlated variables into a smaller number of uncorrelated variables.

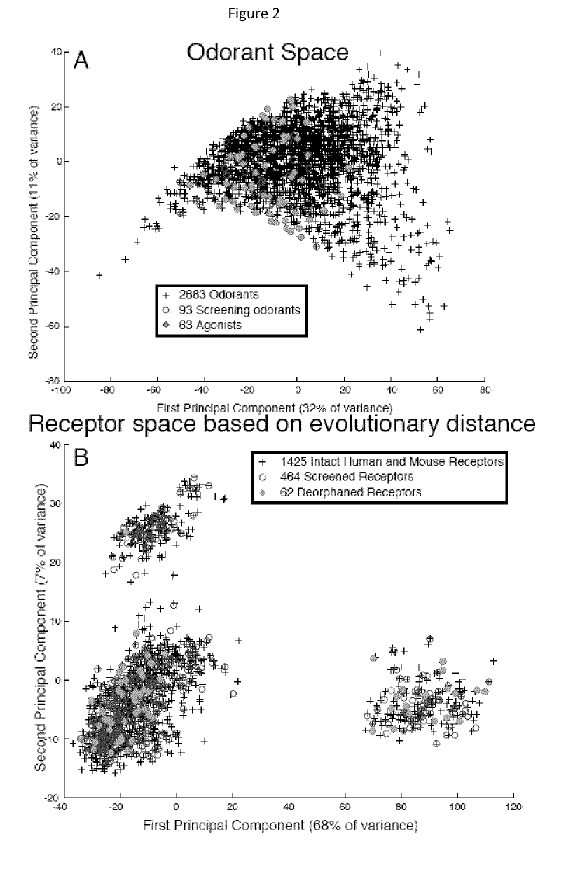

To examine the bias in the odorant set, a 1664-dimensional space was constructed in which each dimension represents a physicochemical property. 2683 commercially available odorants (Table 5) were plotted in the resulting odorant space. A two dimensional projection of this 1664-dimensional odorant space is shown in FIG. 2A.

To examine the bias in the resulting OR cohort, the distance between 1425 human and mouse ORs was mapped (Niimura et al., Gene 346, 23-8 (2005); herein incorporated by reference in its entirety) (Table 2). A 1425×1425 distance matrix was constructed using the Jukes-Cantor method and visualized in two dimensional space using principal component analysis (FIGS. 2B, 13).

The first principal component (PC1) of odorant space correlates with molecular size (Khan et al., J. Neurosci 27:10015 (2007); herein incorporated by reference in its entirety). FIG. 2A indicates that, although there is dense coverage of the middle of PC1, there is less dense coverage at very large and very small odorants. The criterion of including at least one member of 217 out of the 228 mouse receptor subfamilies insured a broad coverage in receptor space based on full-sequence similarity.

### Physicochemical Odorant Properties Predict Functional Data

Early studies focused on small sets of odorant features, such as carbon chain length and functional group, to explain response variability in the olfactory system. More recent studies have used more quantitative approaches using broader sets of physicochemical descriptors. In one such study, physicochemical descriptors of odorants predicted approximately 35% of the variation in perceived odorant pleasantness (Khan, et al., J Neurosci 27, 10015-23 (2007); herein incorporated by reference in its entirety). This finding indicates that physicochemical descriptors might be useful for predicting earlier stages of olfactory perception. Indeed, two recent studies showed that physicochemical descriptors explain 43 to 72% of the variance in receptor neuron responses (Schmuker et al., Chem Cent J 1, 11 (2007); herein incorporated by reference in its entirety) and, in a meta-analysis, that a set of 32 physicochemical descriptors explained an average of 48% of the variance in neural responses for various olfactory data sets (Haddad et al., Nature Methods 5:425 (2008); herein incorporated by reference in its entirety). The experiments described herein examined how well several proposed metrics predict the functional data (FIG. 3A).

In the data set, carbon number alone described a very small portion of the variance in receptor response (r=0.07, p<0.04). Functional group descriptors (r=0.42, p<0.0001) explained nearly as much variance as all 1664 tested descriptors (r=0.43, p<0.0001). Haddad et al. (supra) proposed a set of 32 descriptors, a subset of the 1664 used here, that described a large portion of the variance in a meta-analysis of 8 olfactory data sets. These descriptors outperformed carbon number, the set of functional group descriptors, and the full set of descriptors (r=0.59, p<0.0001). Haddad et al. optimized the set of 32 descriptors using a meta-analysis including data from different model organisms, different measurement techniques, and different levels of the olfactory system. The present experiments similarly optimized the descriptor set using a greedy optimization algorithm using a leave-10-out cross-validation scheme, but only on data from the present assay system. On average, this technique explained 60% of the variance in the left-out dataset (r=0.77). To verify that this result was not due to chance, each descriptor vector was randomly shoveled to create a new set of randomized descriptors from the same distribution. Real descriptors significantly outperformed shuffled descriptors [shuffled r=0.55, t(23)=9.09, p<0.0001]. Having validated this technique, the algorithm was applied to the entire dataset. To reduce overfitting, descriptors were chosed that explained the most variance averaged over ten divisions of the dataset, resulting in a set of 18 descriptors that explain over 62% of the variance in the dataset (r=0.79, p<0.0001) (FIG. 3B-C). In other words, much of the variation in OR responses can be explained by a fairly small set of physicochemical descriptors.

### Receptor Sequence Predicts Functional Data

OR genes are classified into families and subfamilies based on sequence alignment of the full-length proteins. Because of the paucity of functional data regarding these receptors, it is unclear if these divisions correspond to functional variability (Abaffy et al., J Neurochem 97, 1506-18 (2006); herein incorporated by reference in its entirety). In other words, the assumption that full-length protein sequence variability predicts functional variability has not been tested on a comprehensive set of receptors.

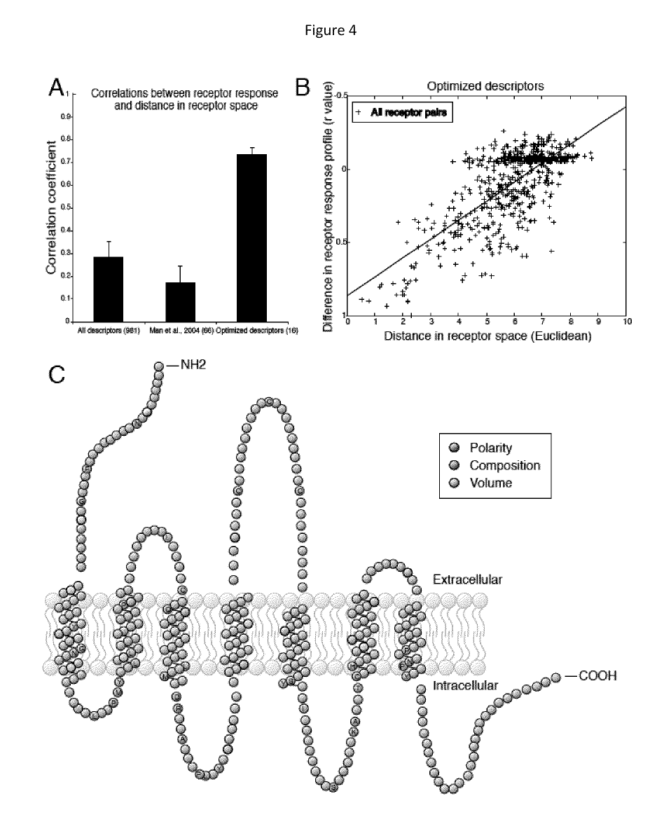

Here this is tested by examining how well the properties of amino acids residues predict the functional data (FIG. 4A).

As with odorants, a set of descriptors for ORs was constructed. Using a multiple alignment of 1425 intact mouse and human ORs (Table 2), amino acid properties (polarity, composition, and volume) were calculated as defined by Grantham (Grantham, Science 185, 862-4 (1974); herein incorporated by reference in its entirety) for 327 amino acid residues common to at least 10% of the ORs. The entire set of descriptors explained only 7% of the variance (r=0.28, p<0.0001). Recently, Man et al. (PLoS ONE 2, e682 (2007); herein incorporated by reference in its entirety) proposed that comparison of predicted ligand-binding residues predicted functional variation more accurately than full-length comparisons. Restricting the set of descriptors to the 66 properties of 22 predicted binding site residues (Man et al., Protein Sci 13:240 (2004); herein incorporated by reference in its entirety), however, did not improve prediction of functional variation (r=0.17, p<0.0001). The descriptor set was optimized using a greedy optimization algorithm using a leave-10-out cross validation scheme. On average, this technique explained 40% of the variance in the left-out dataset (r=0.63). To verify that this result was not due to chance, each descriptor vector was randomly shuffled to create a new set of randomized descriptors from the same distribution. As expected, real descriptors significantly outperformed shuffled descriptors [r=0.18, t(12)=8.53, p<0.0001]. Having validated this technique, the algorithm was applied to the entire dataset. To reduce overfitting, descriptors that explained the most variance averaged over ten divisions of the dataset were chosen, resulting in a set of 16 descriptors that explain over 53% of the variance in the dataset (r=0.73, p<0.0001) (FIGS. 4B and C, also see Table 5).

### Breadth of Tuning

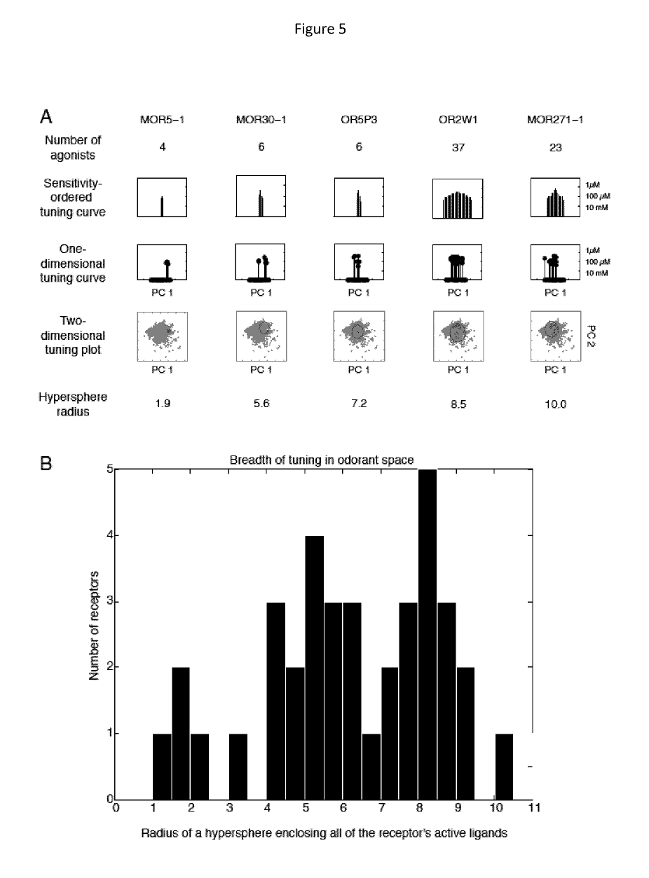

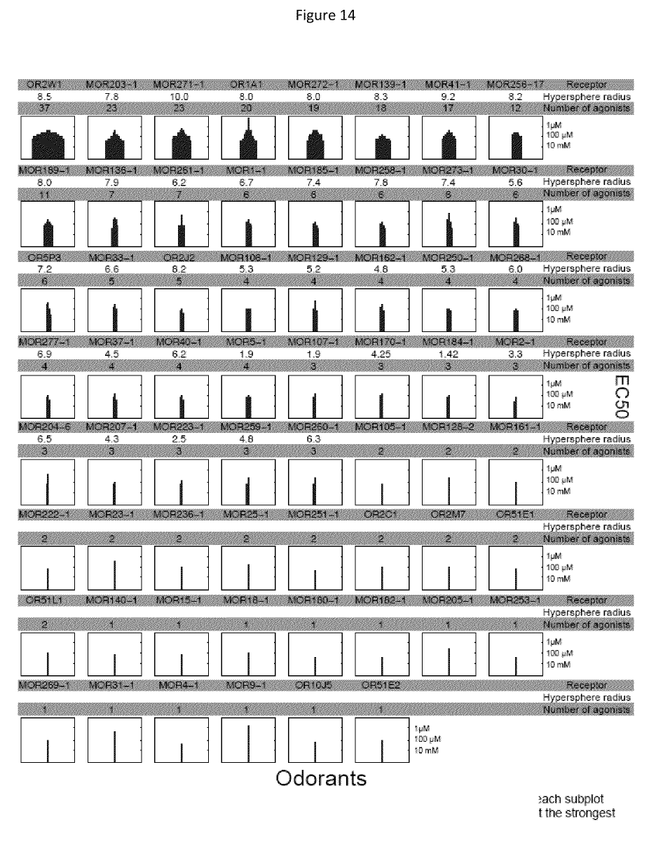

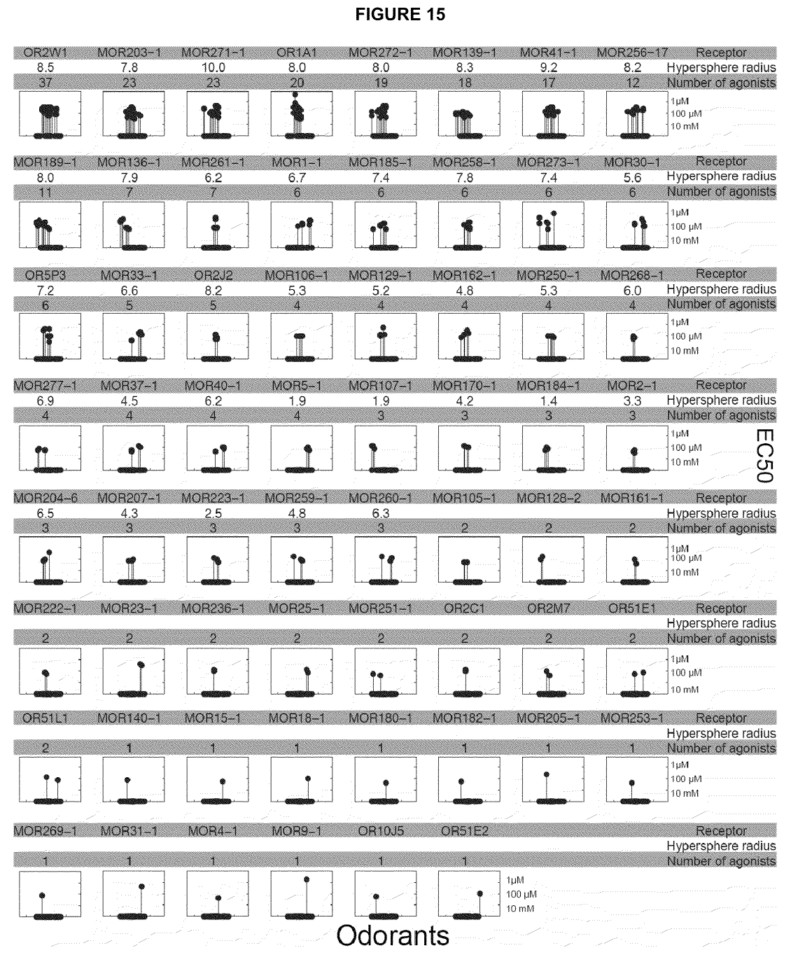

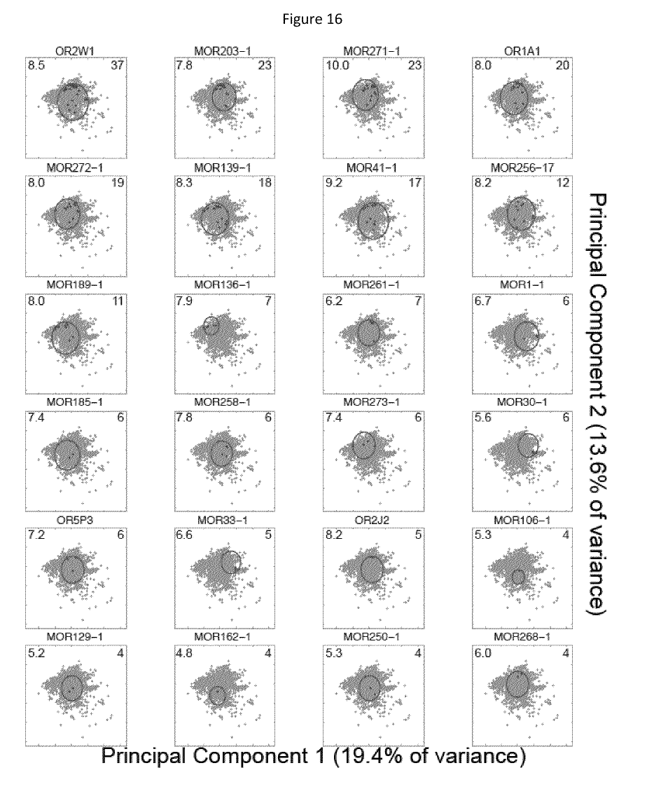

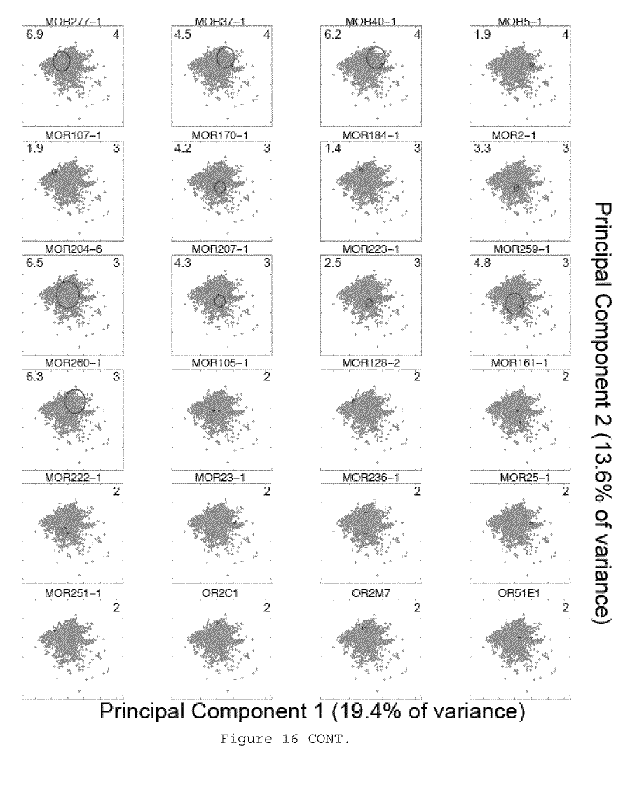

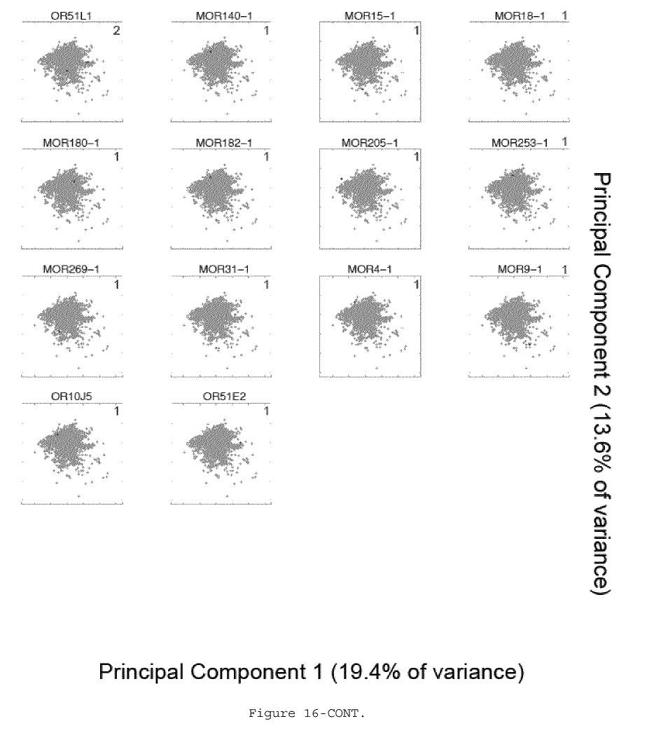

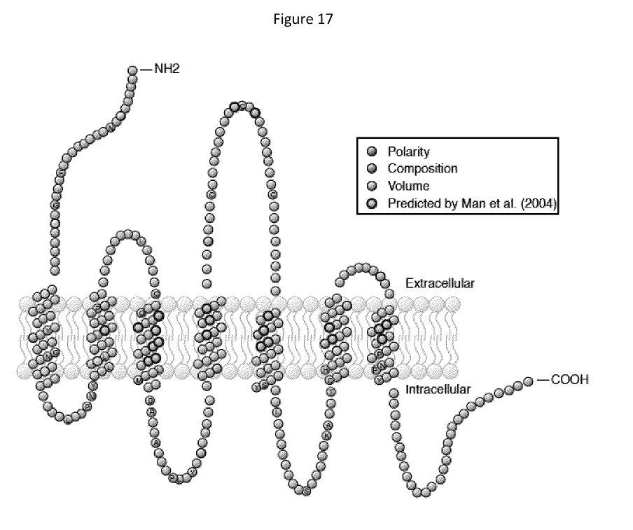

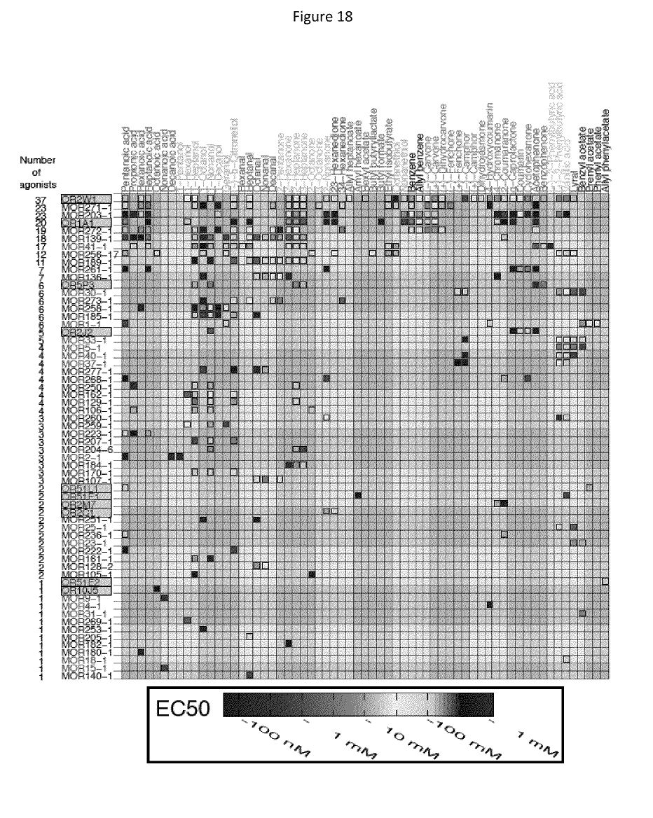

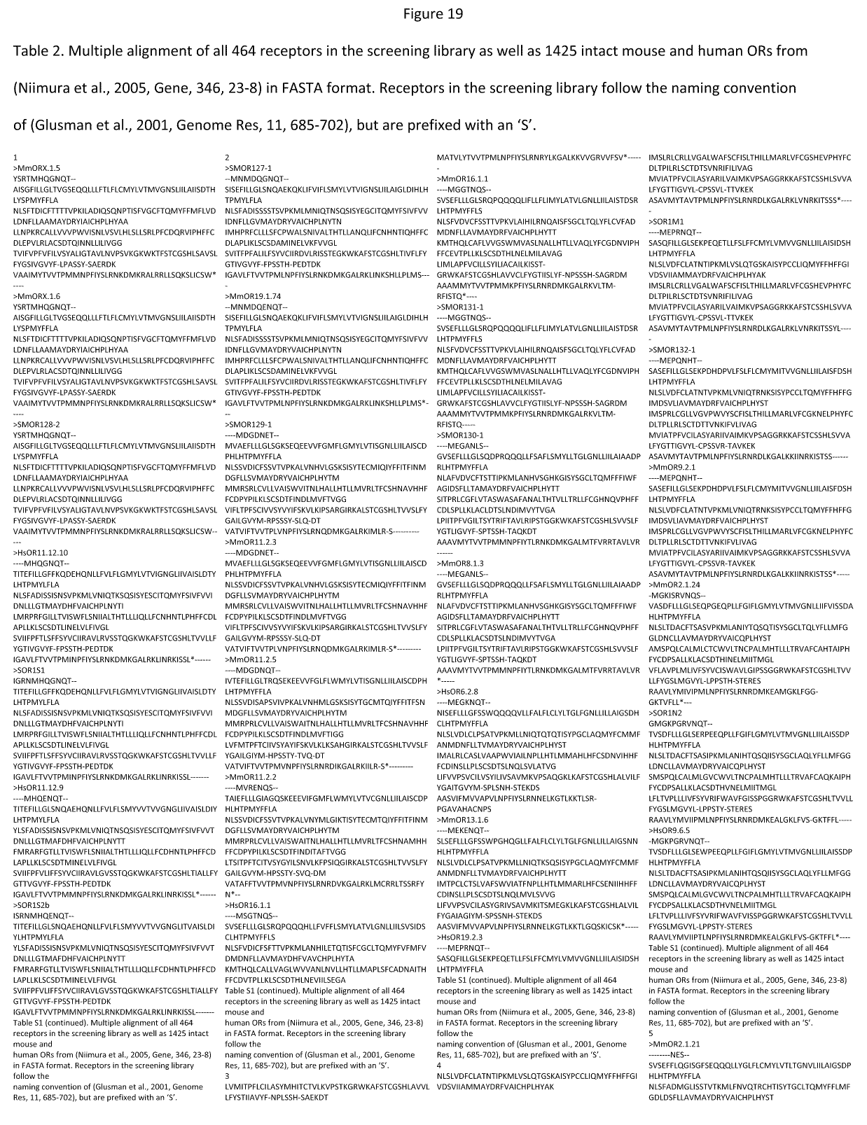

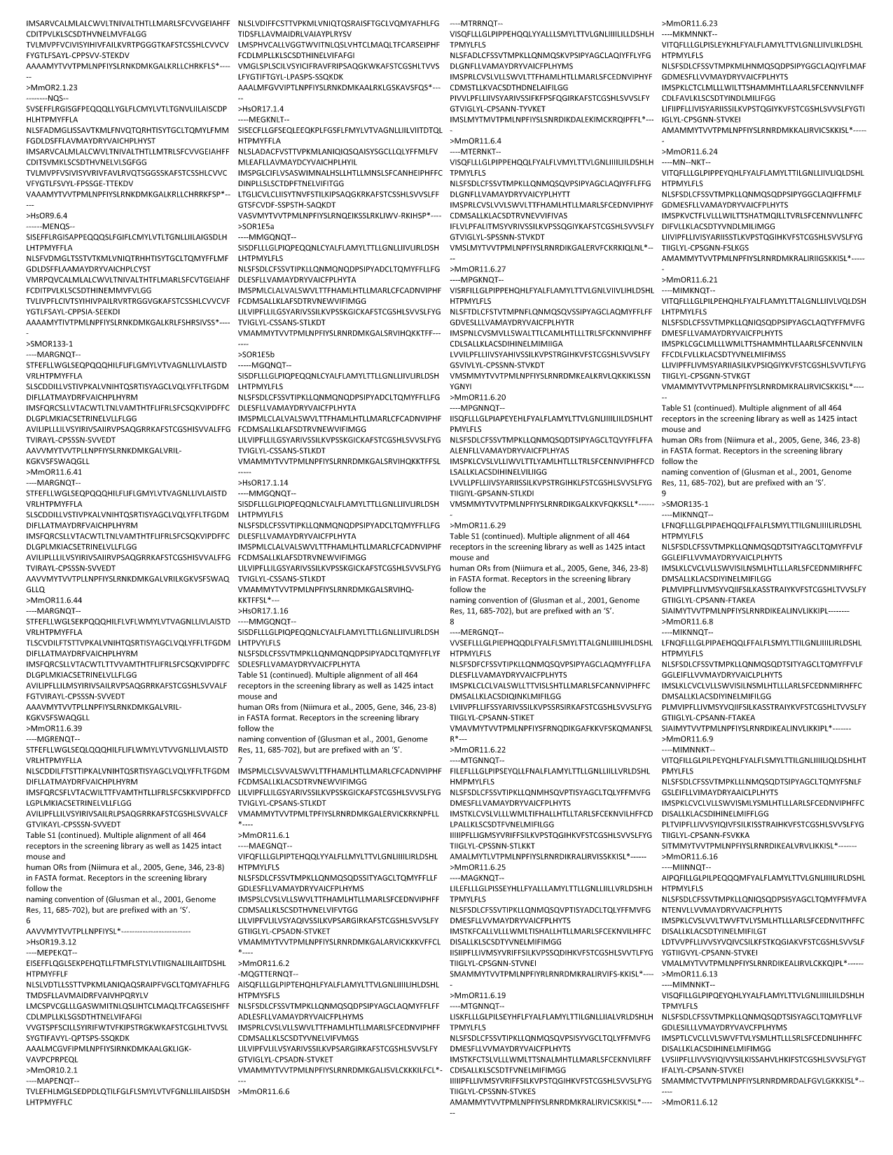

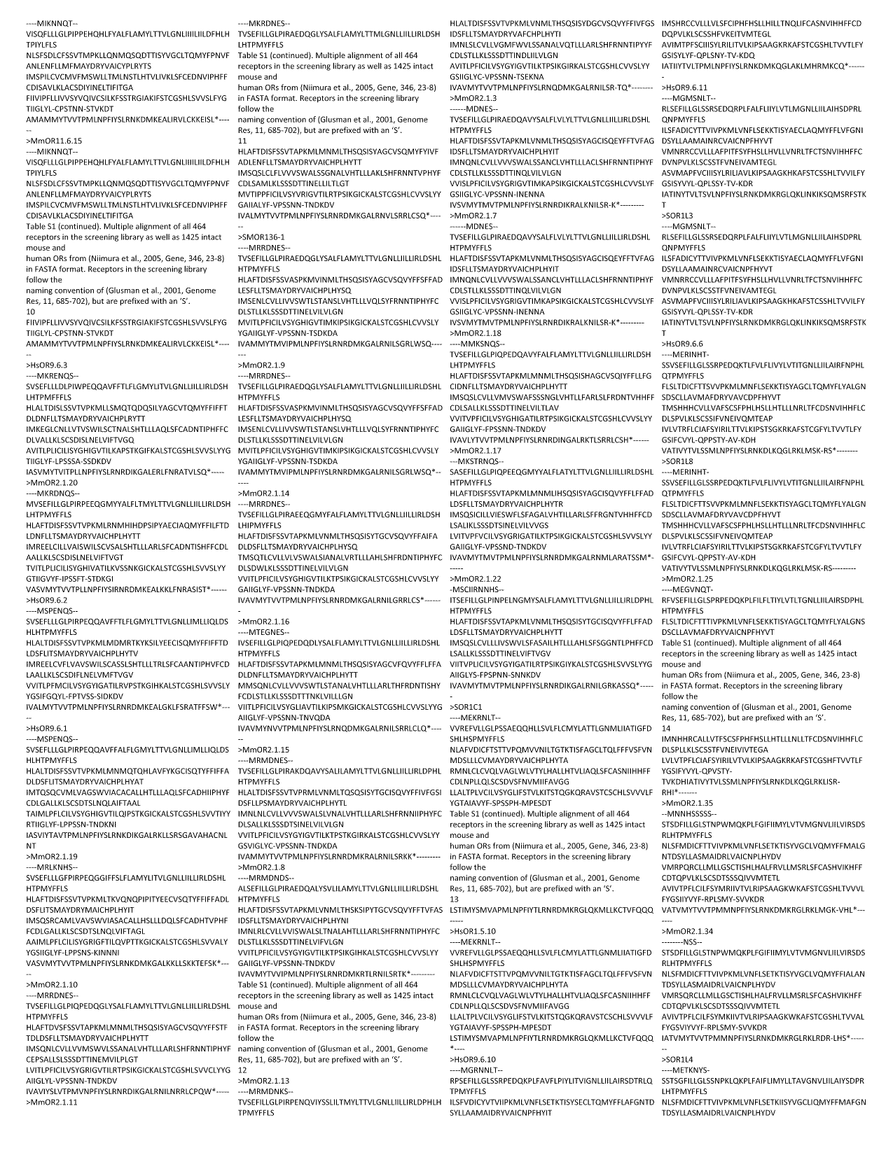

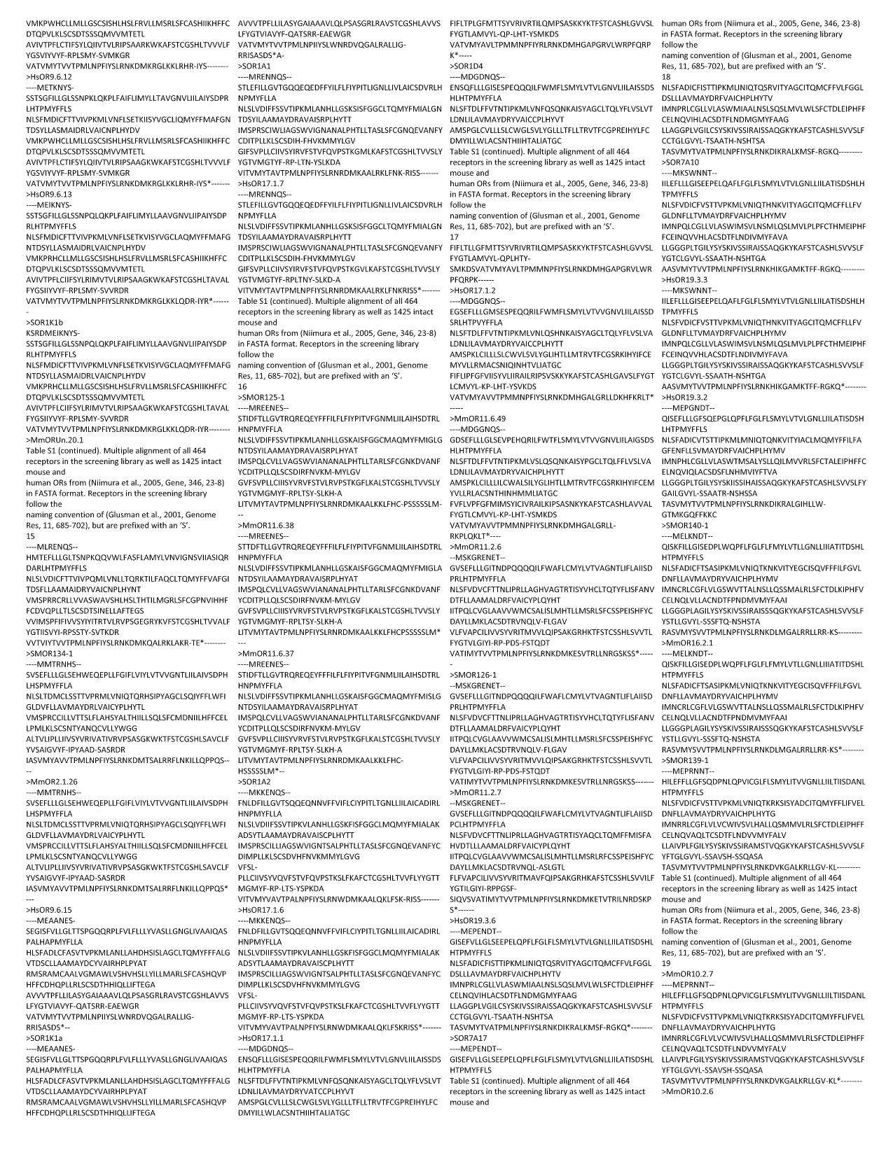

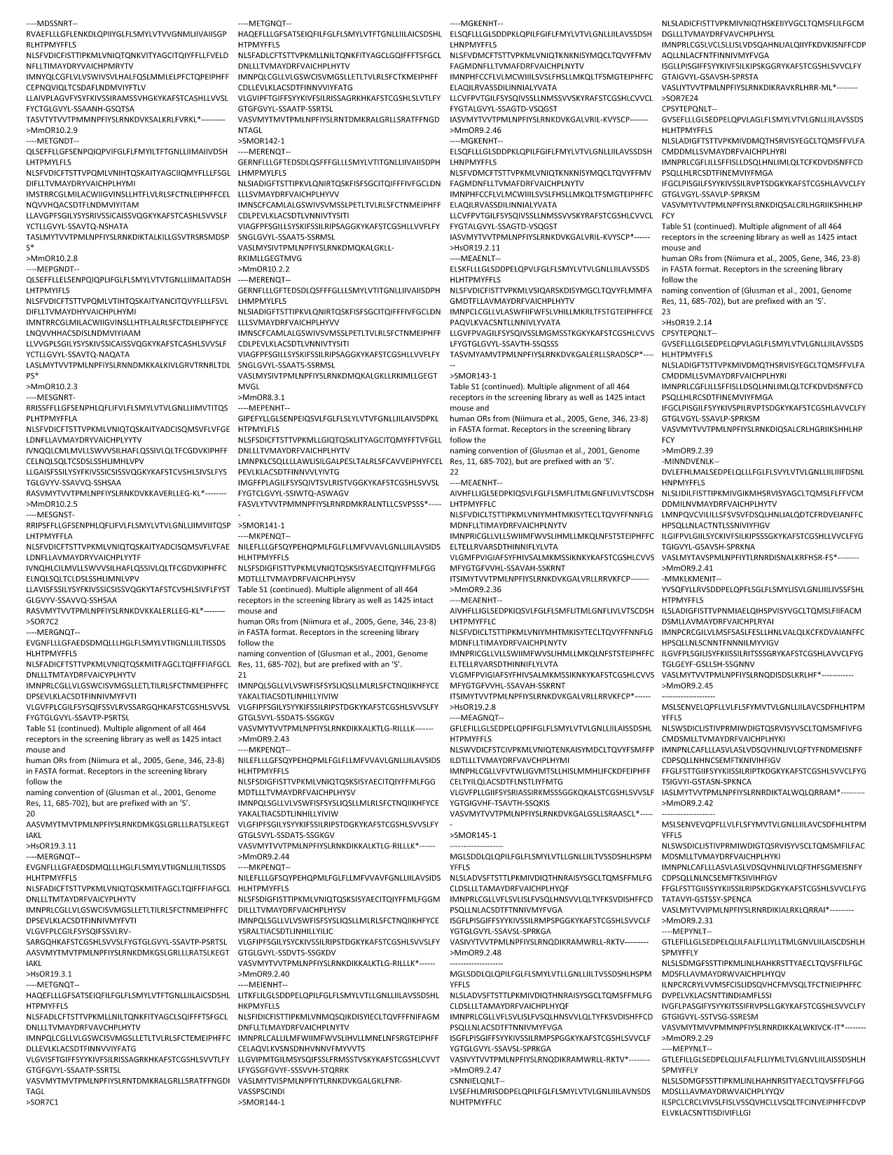

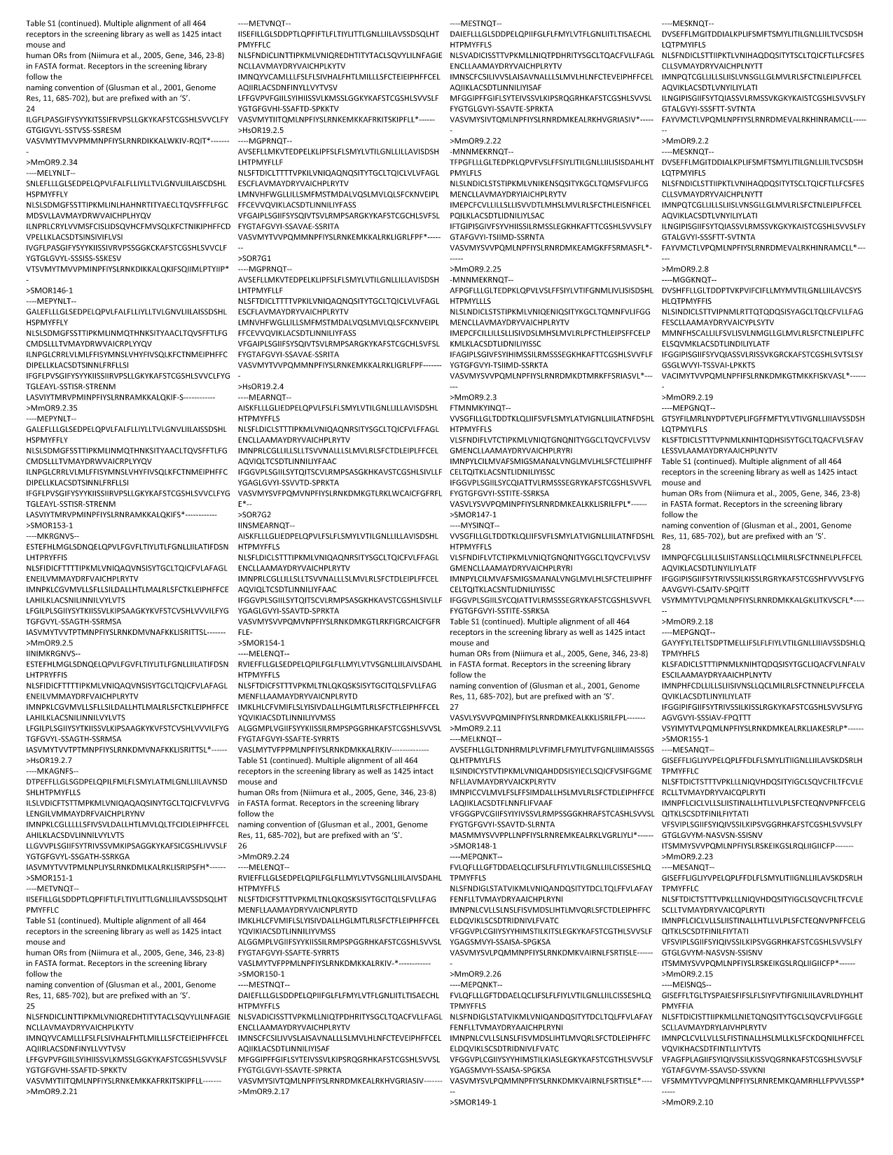

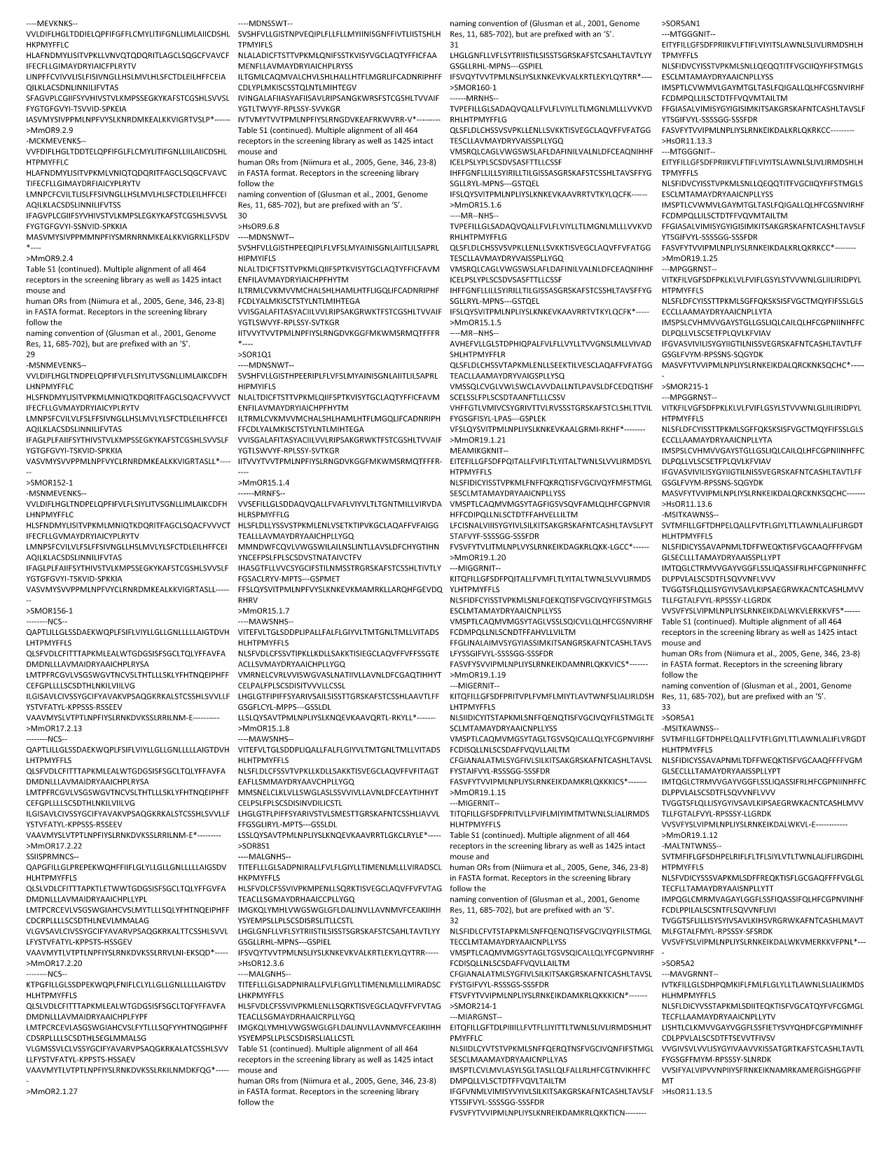

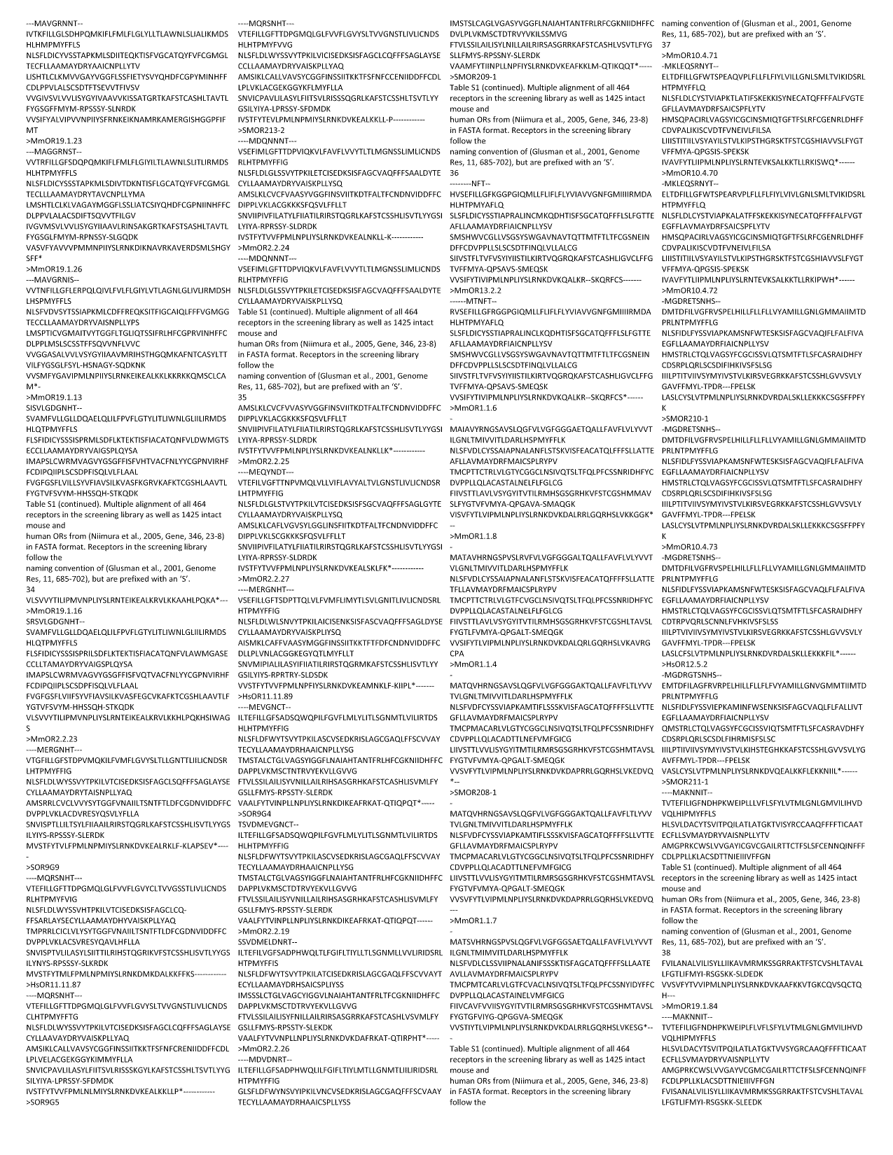

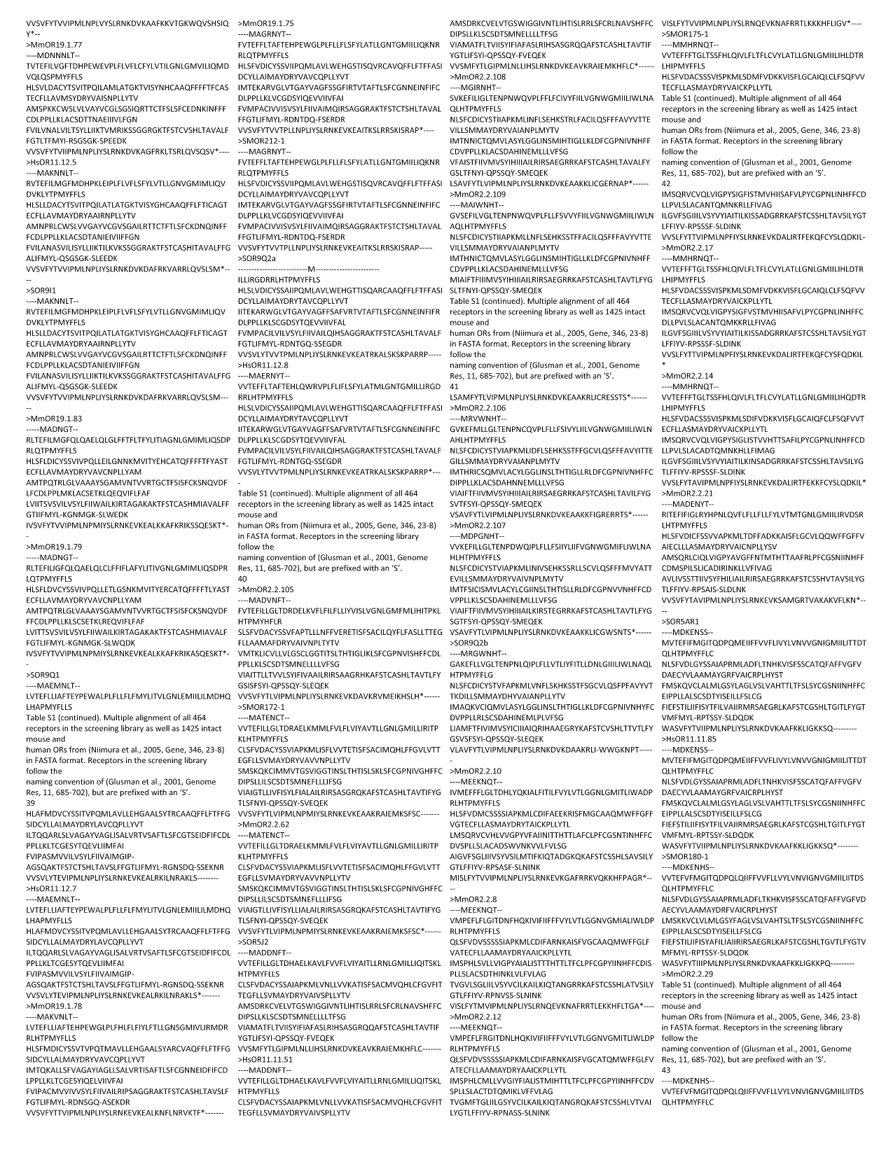

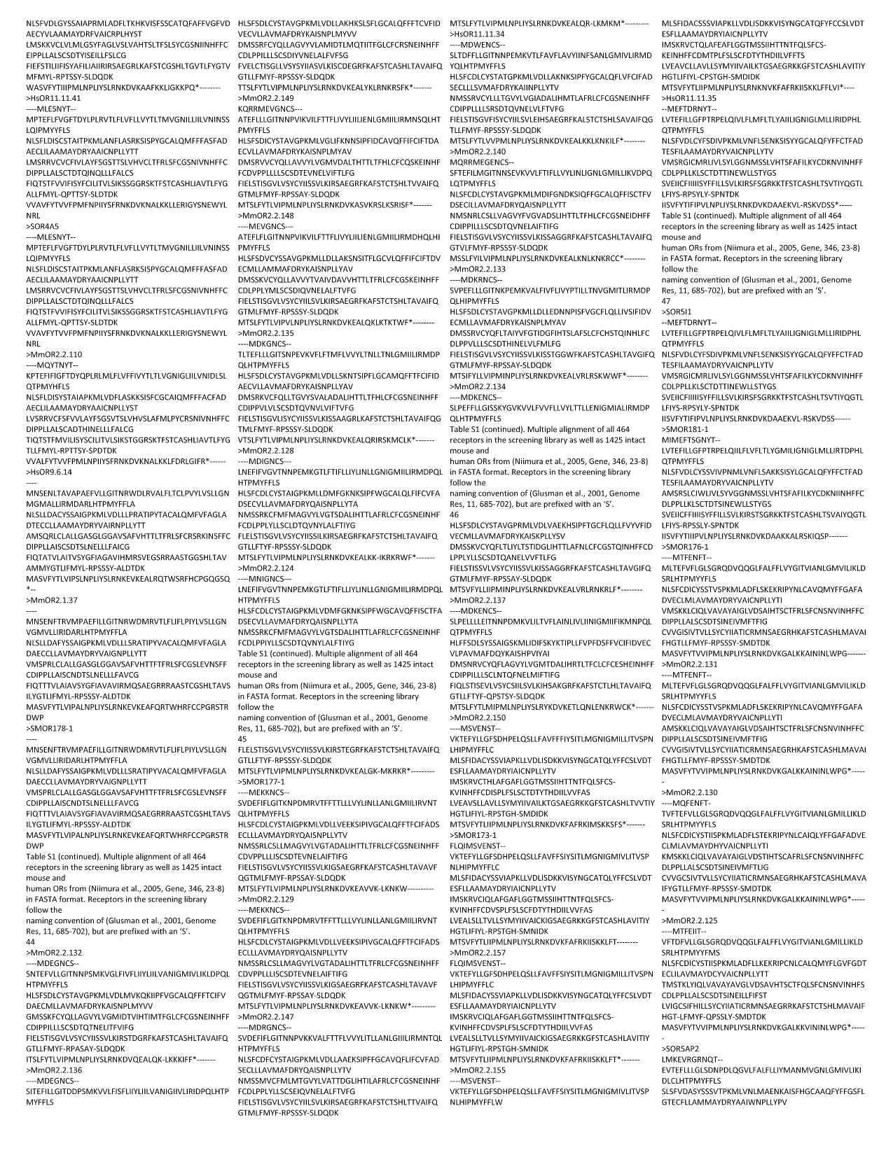

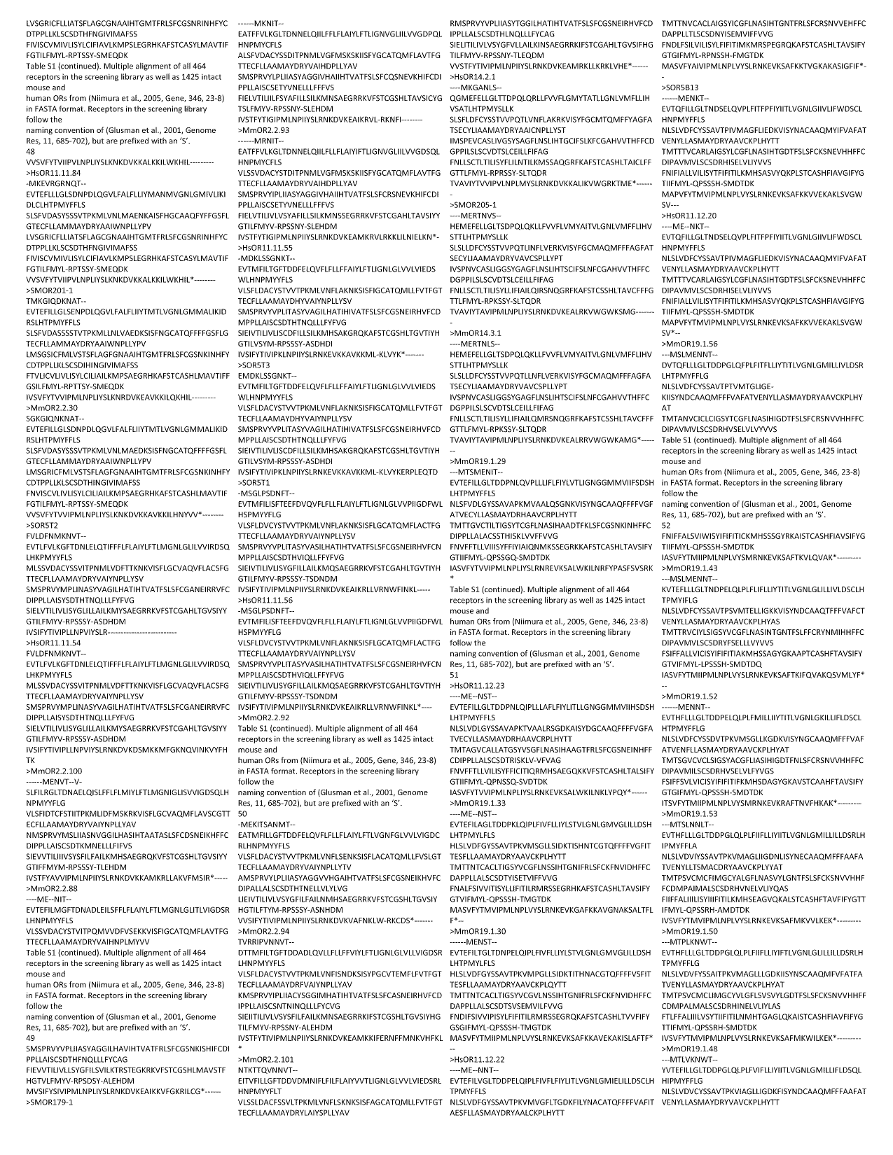

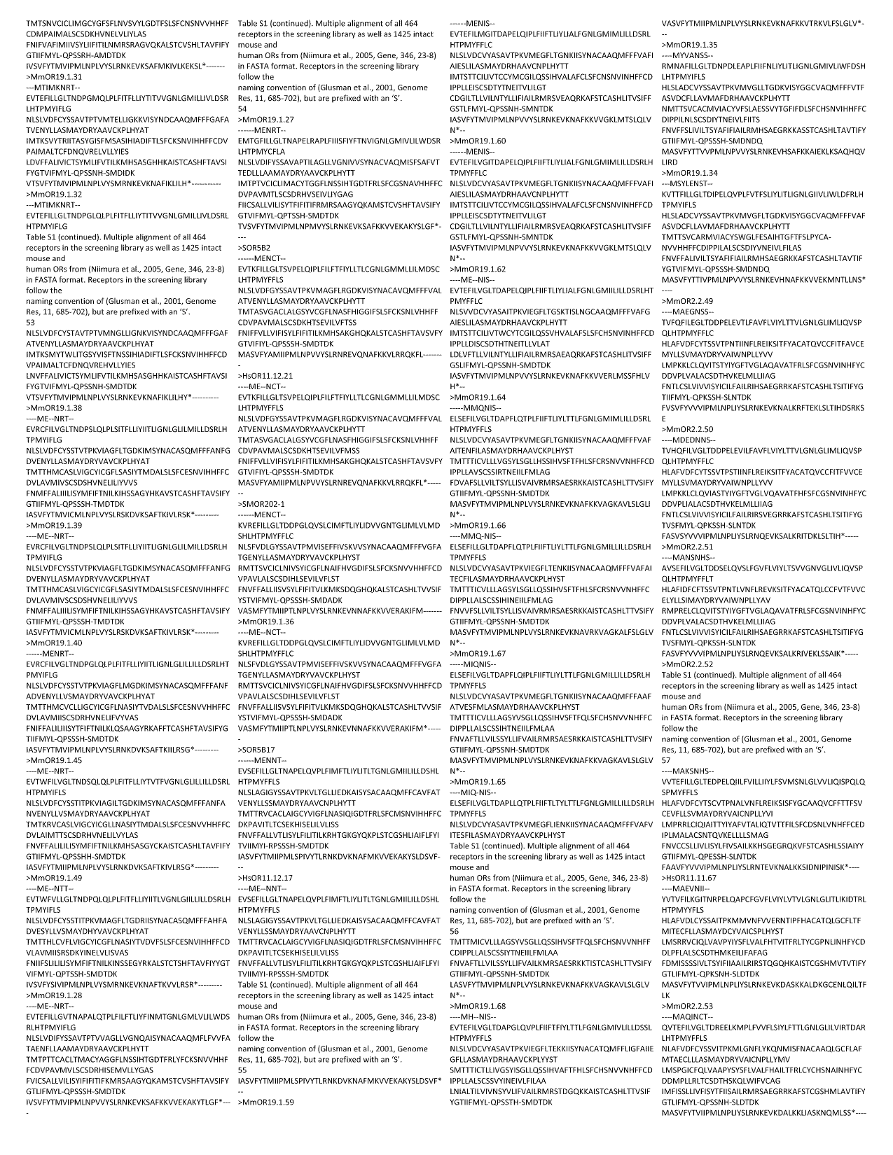

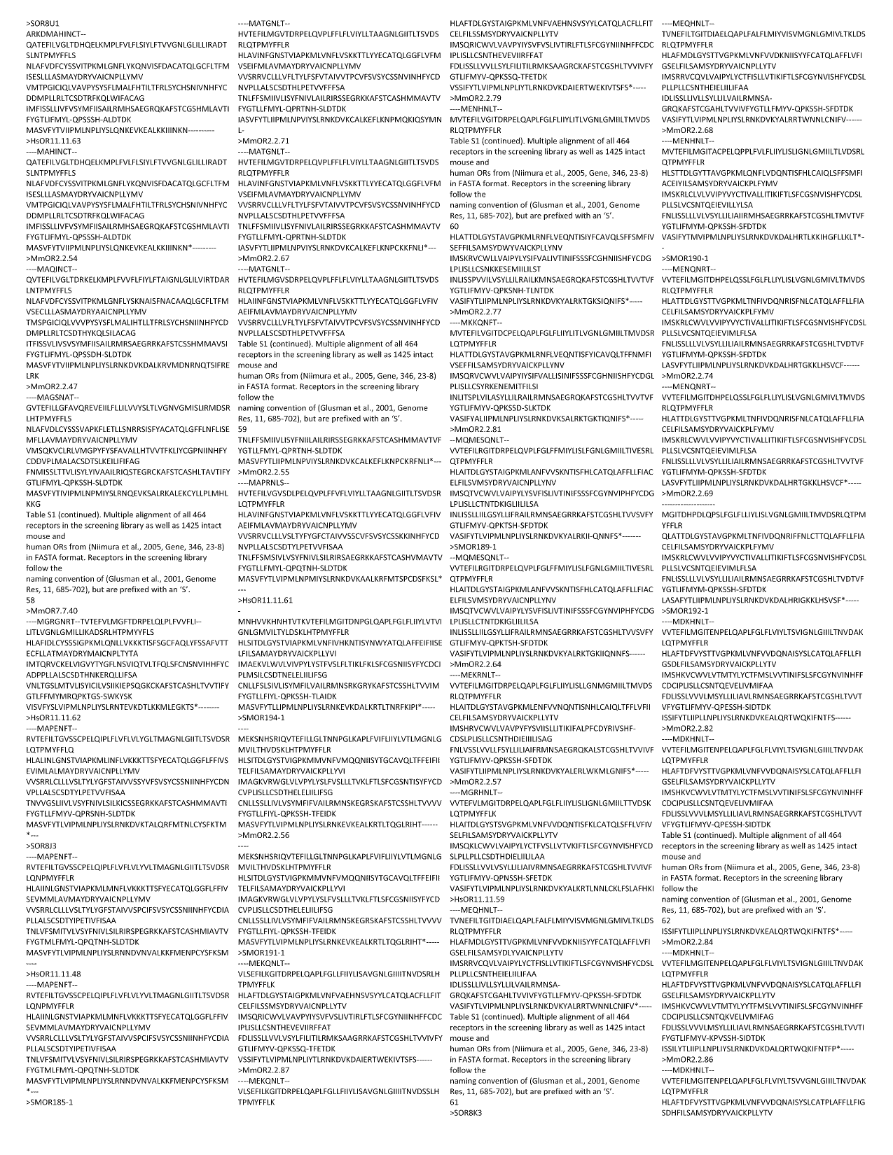

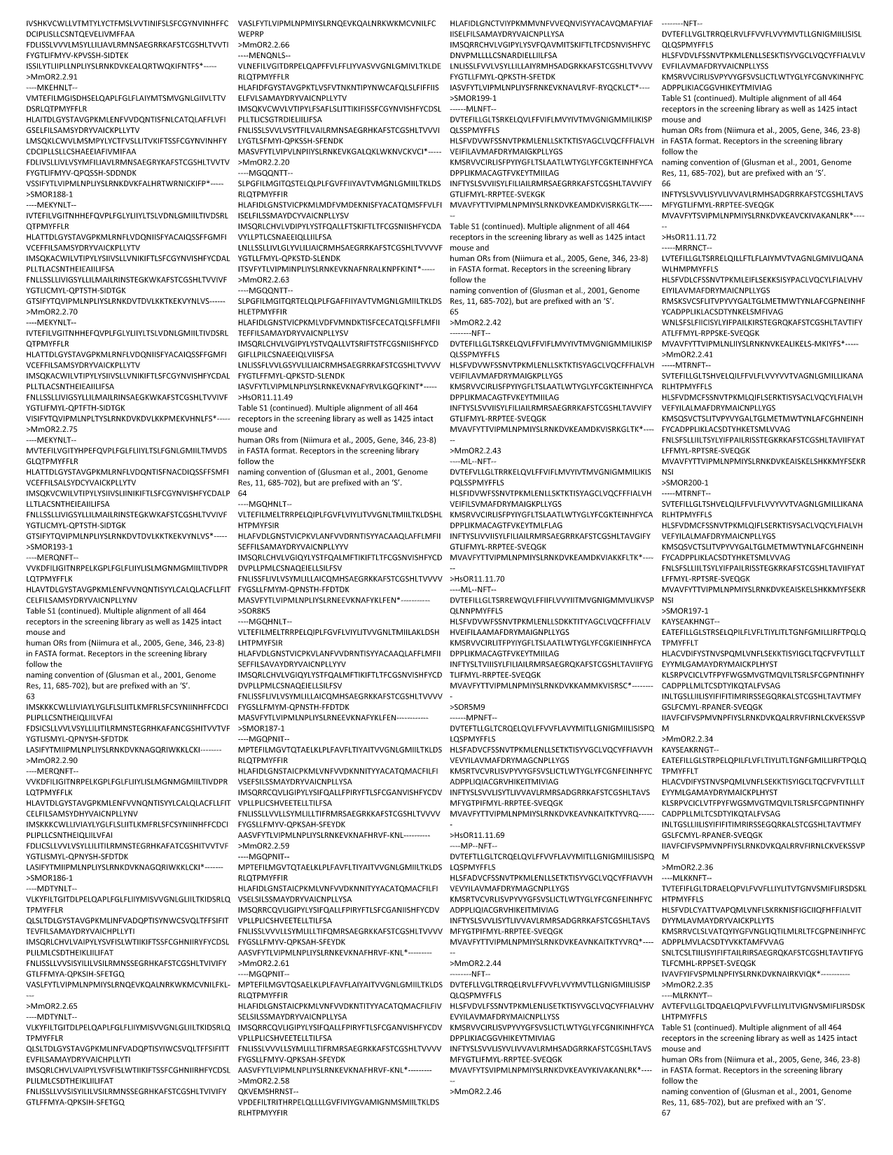

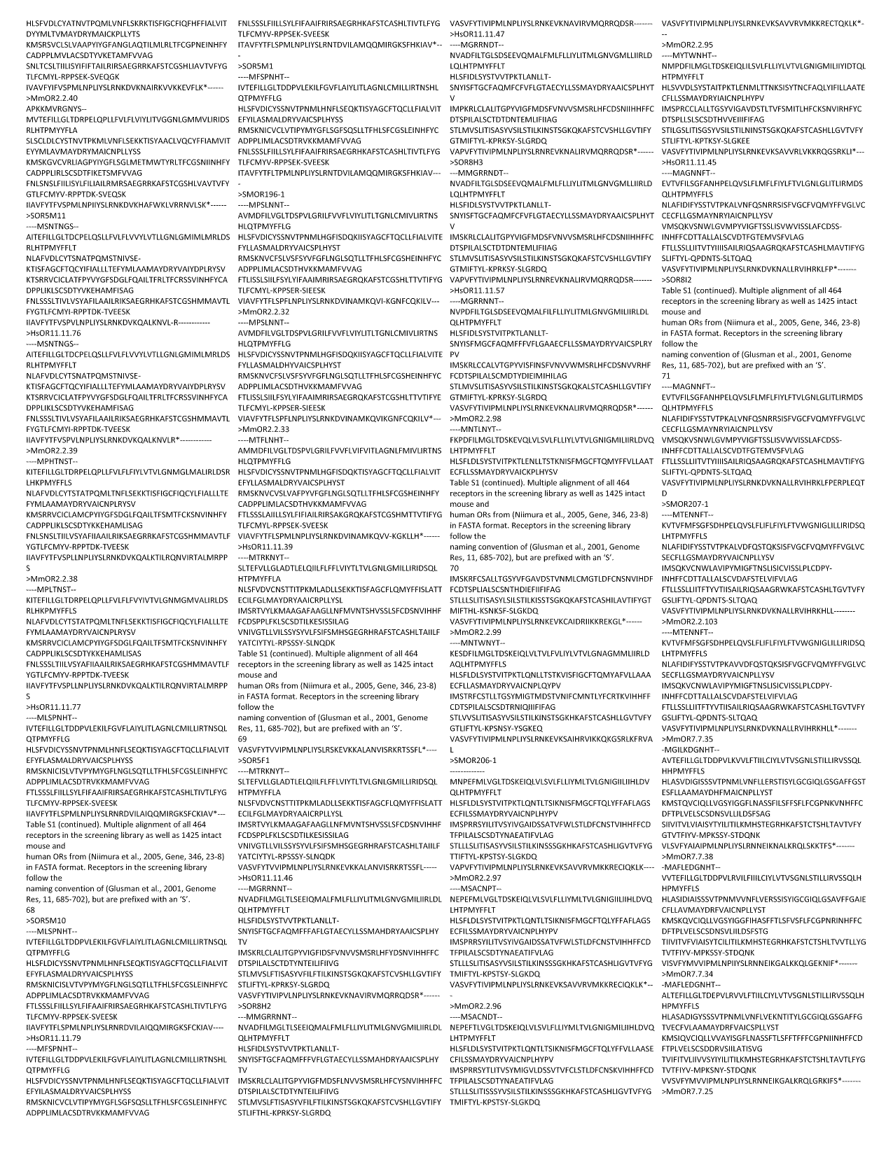

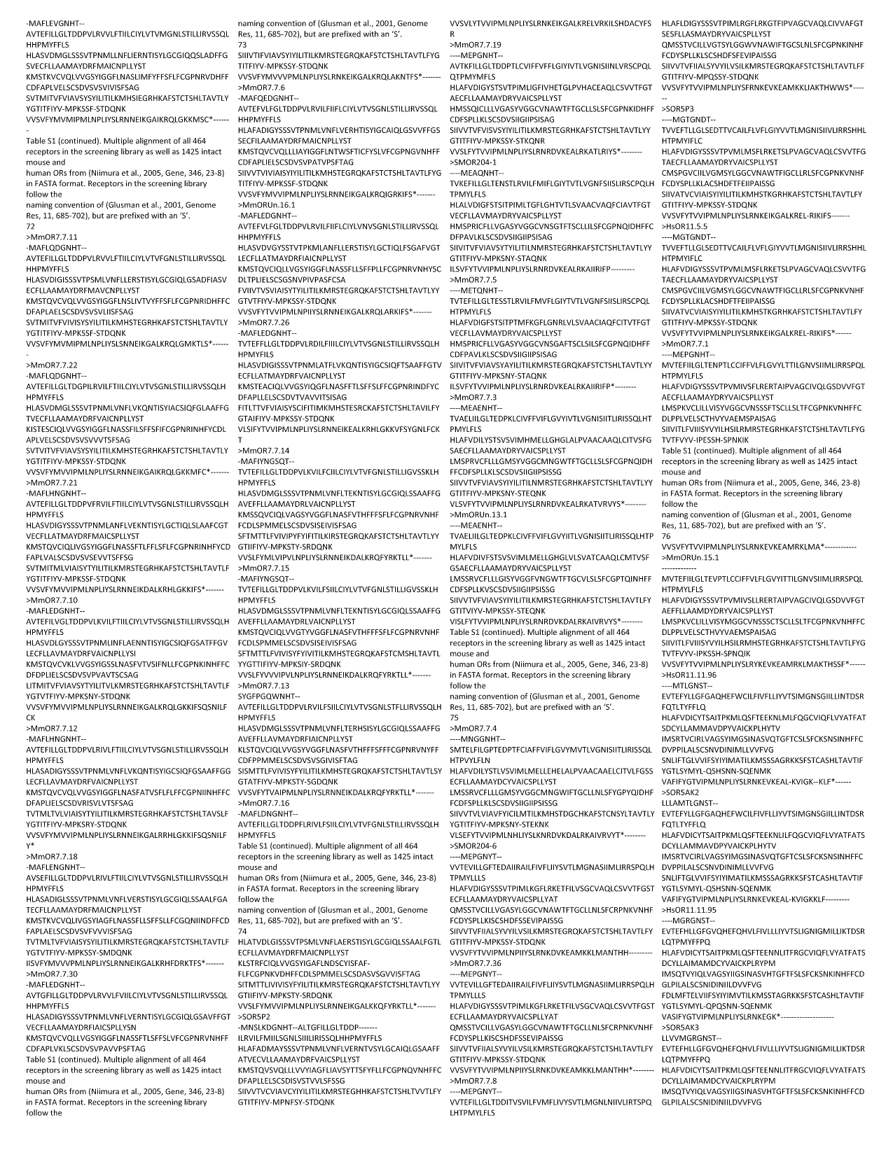

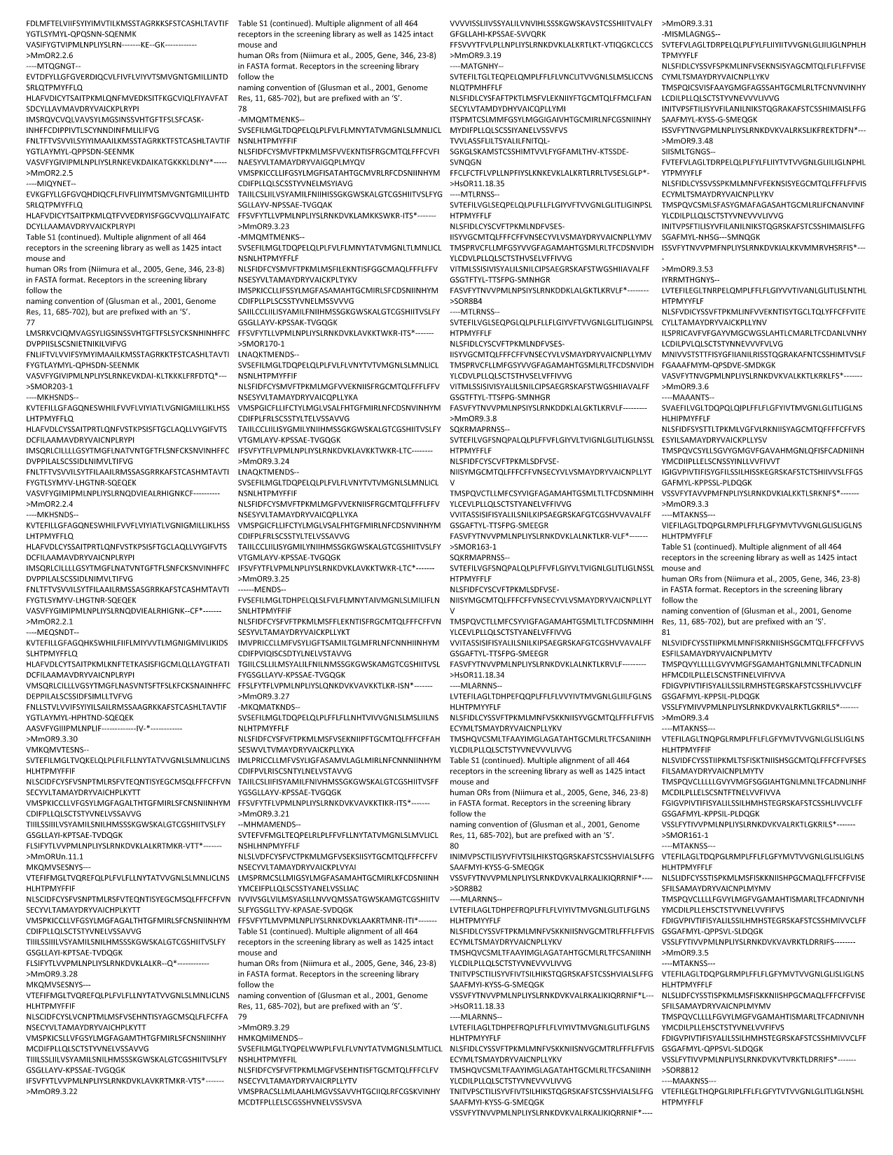

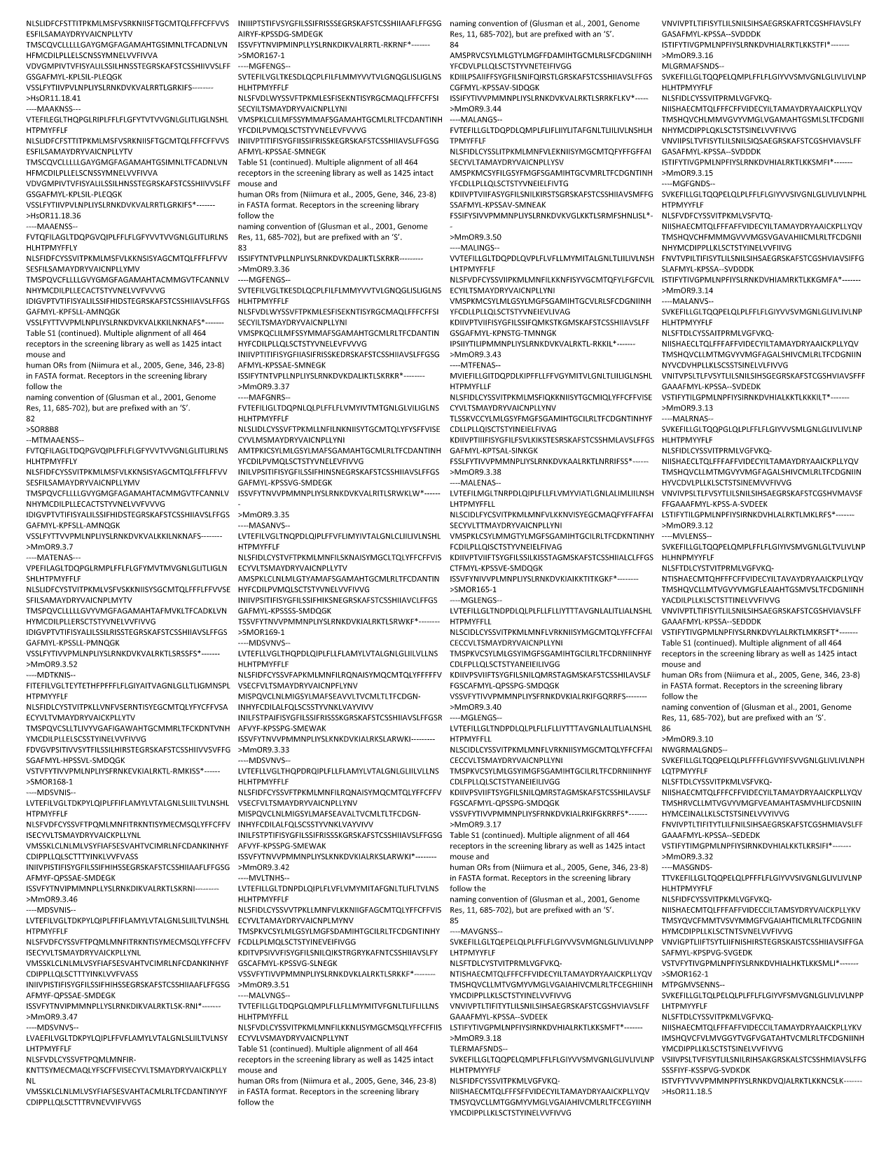

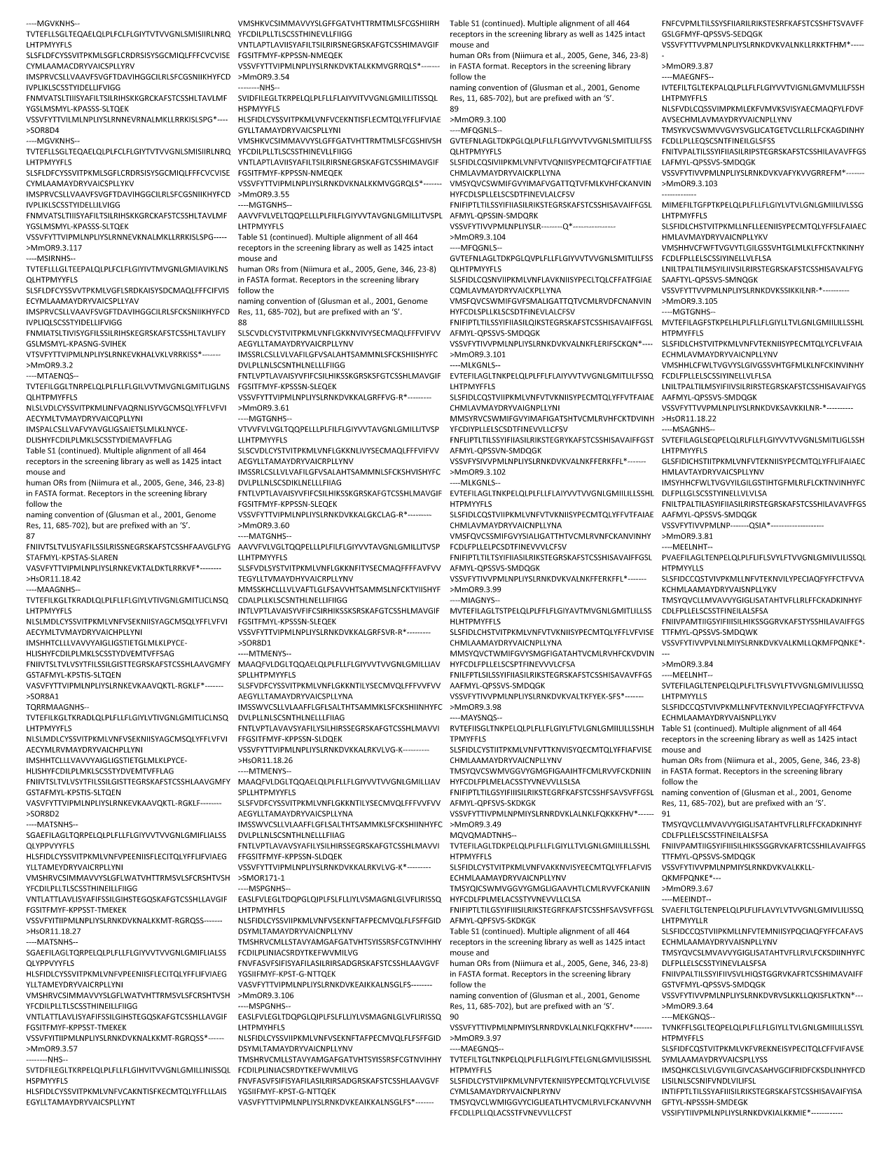

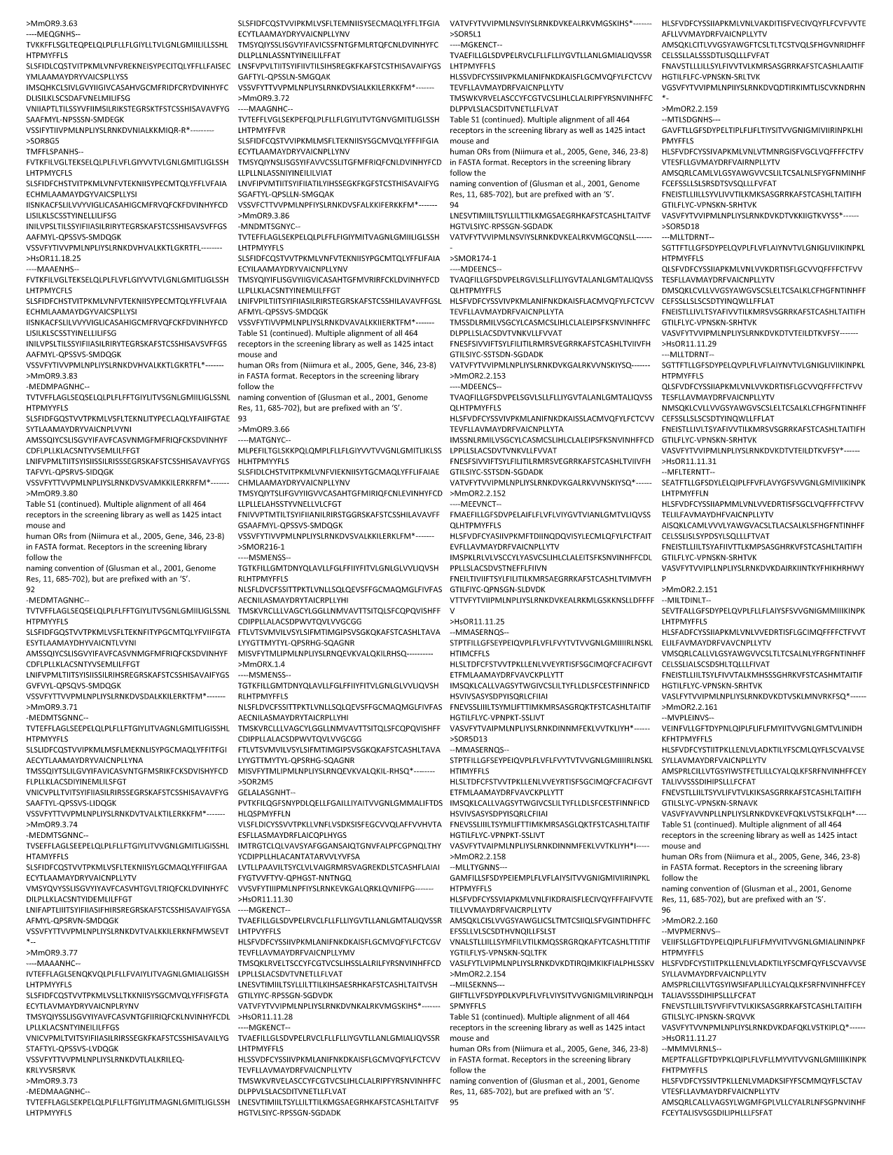

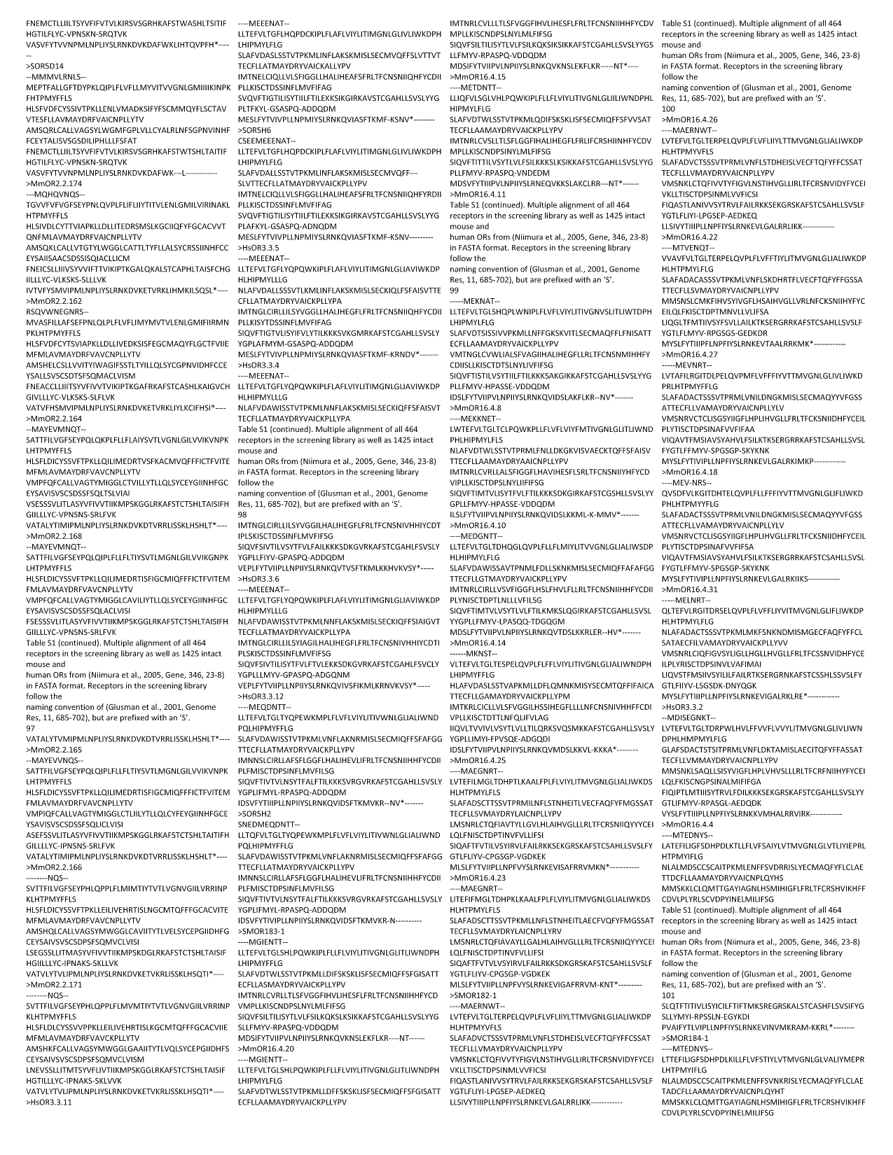

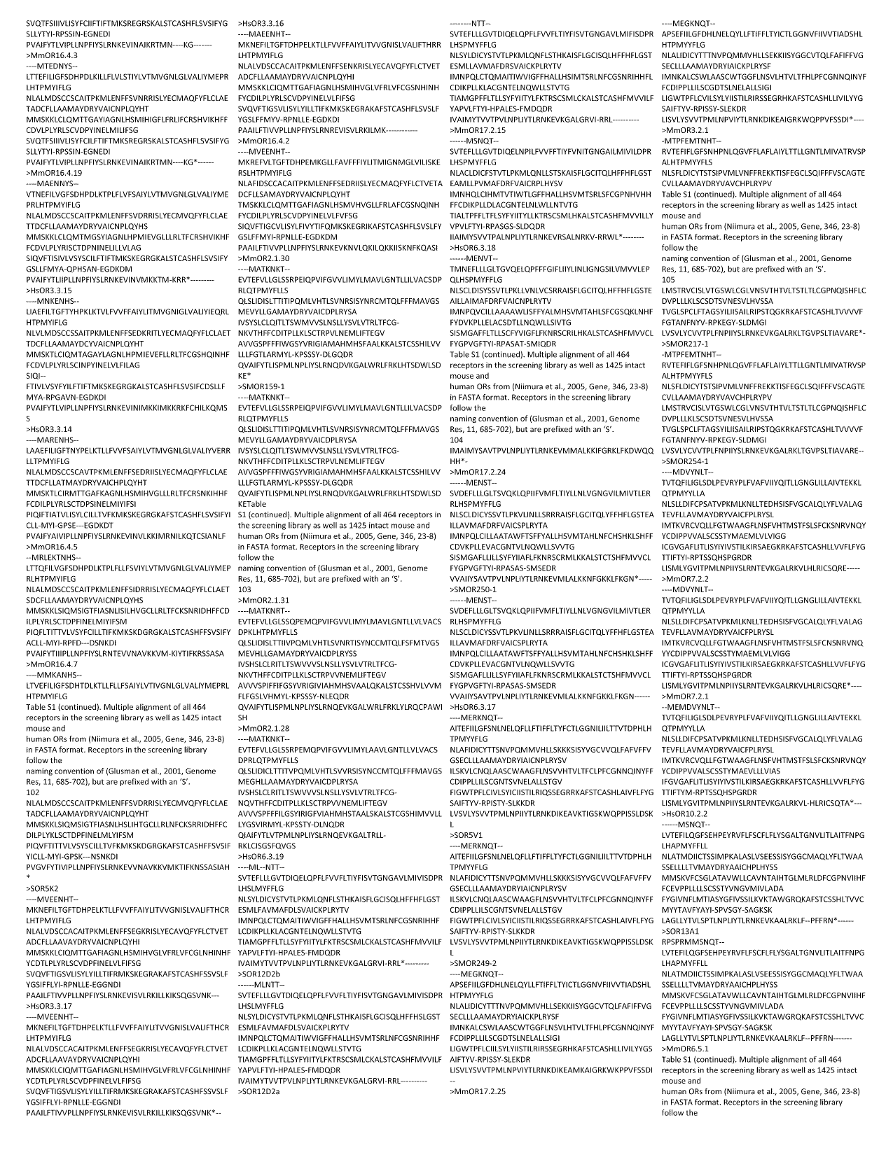

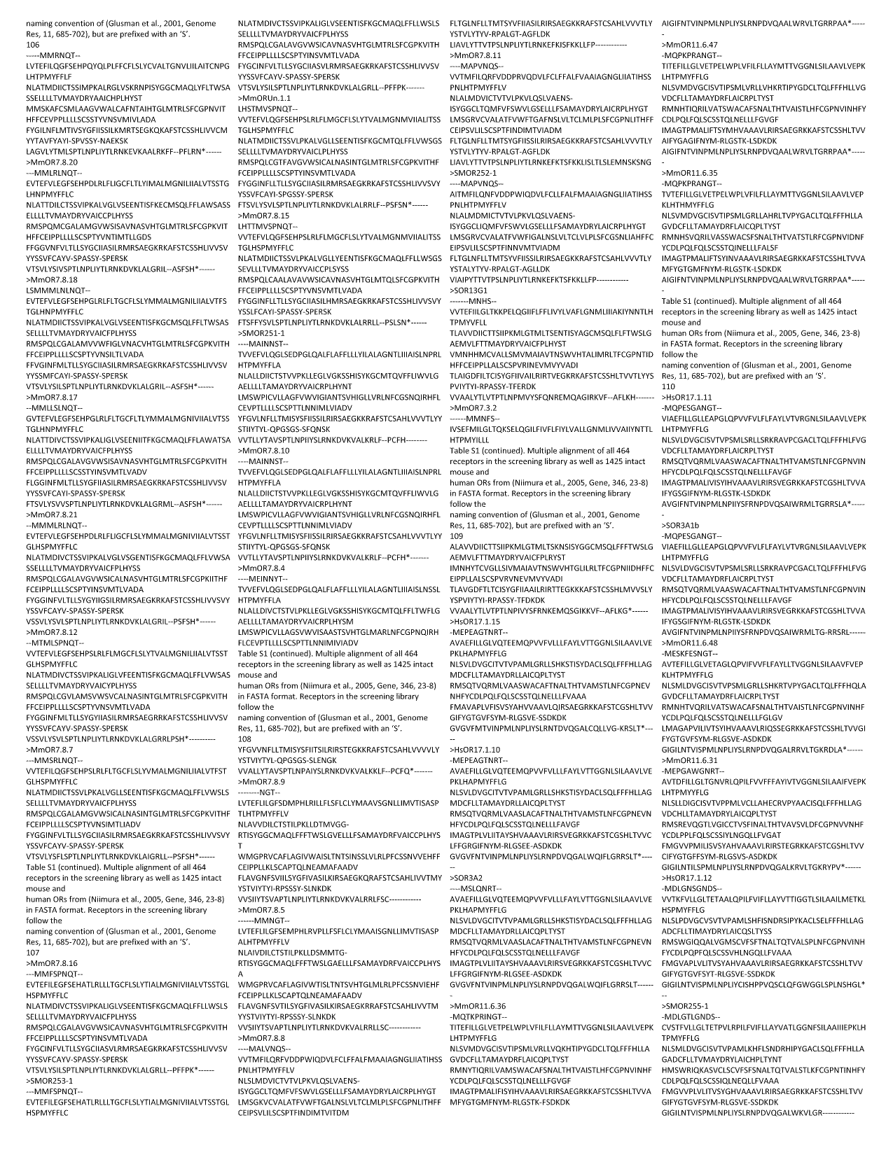

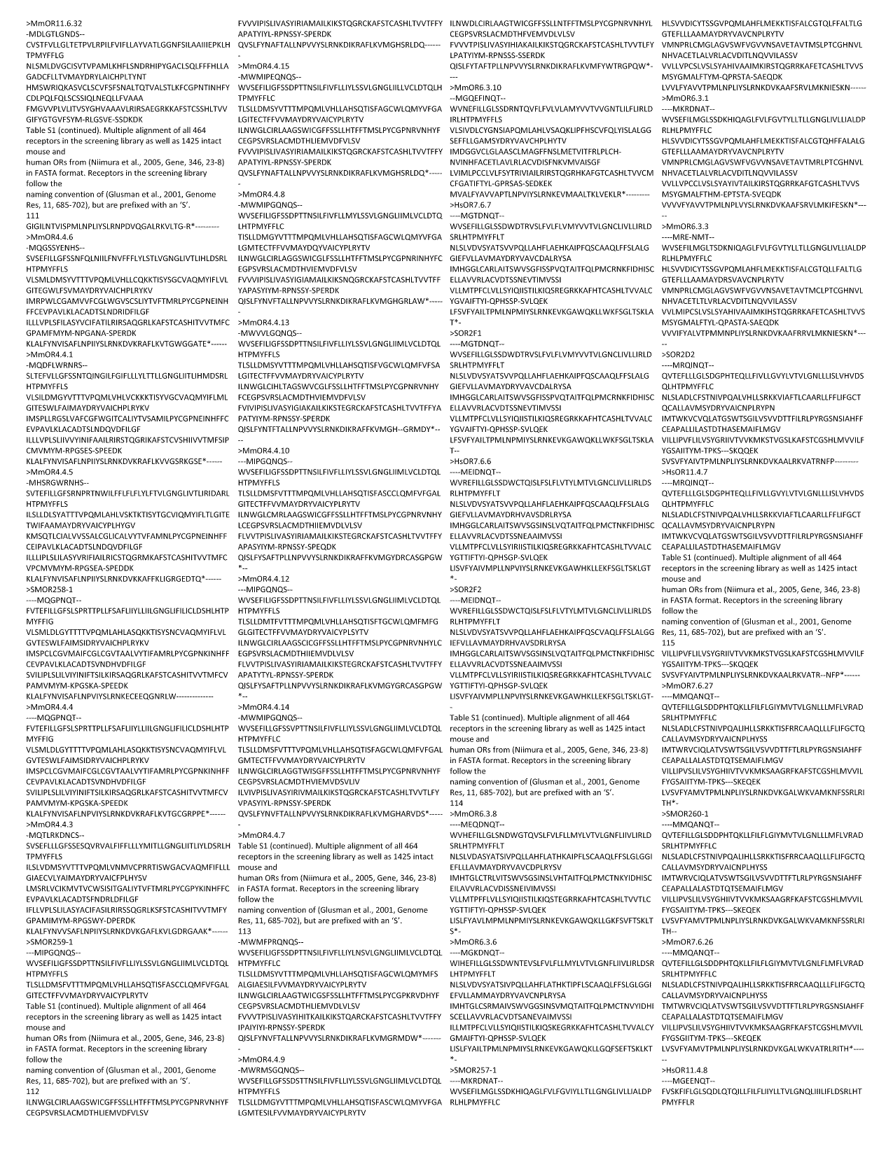

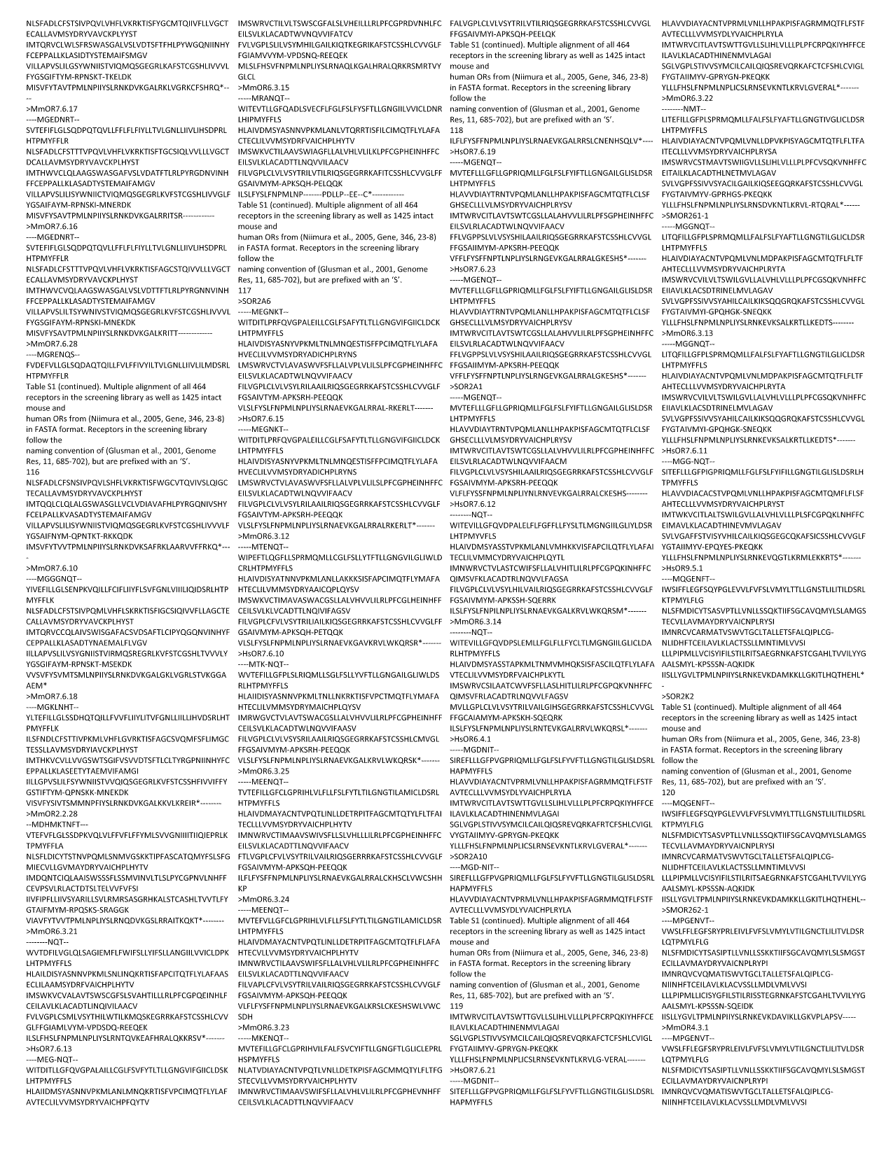

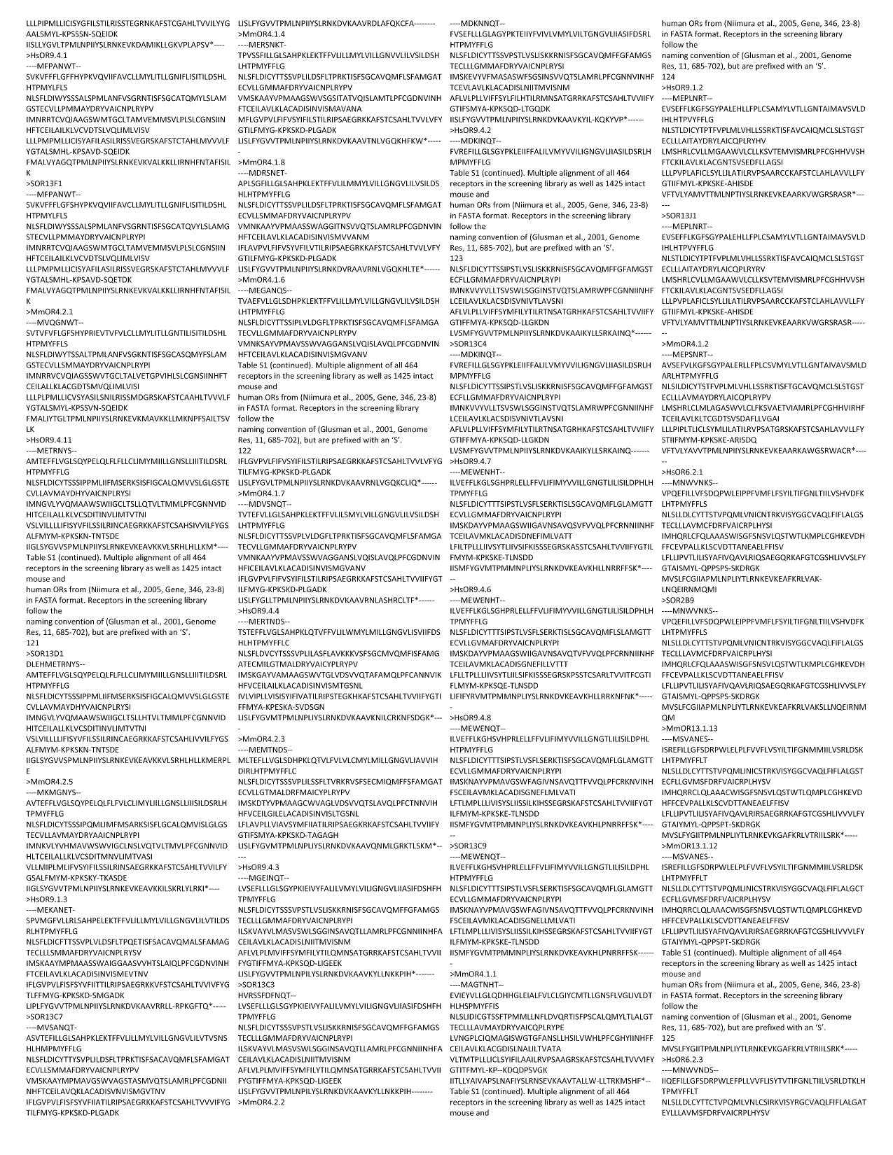

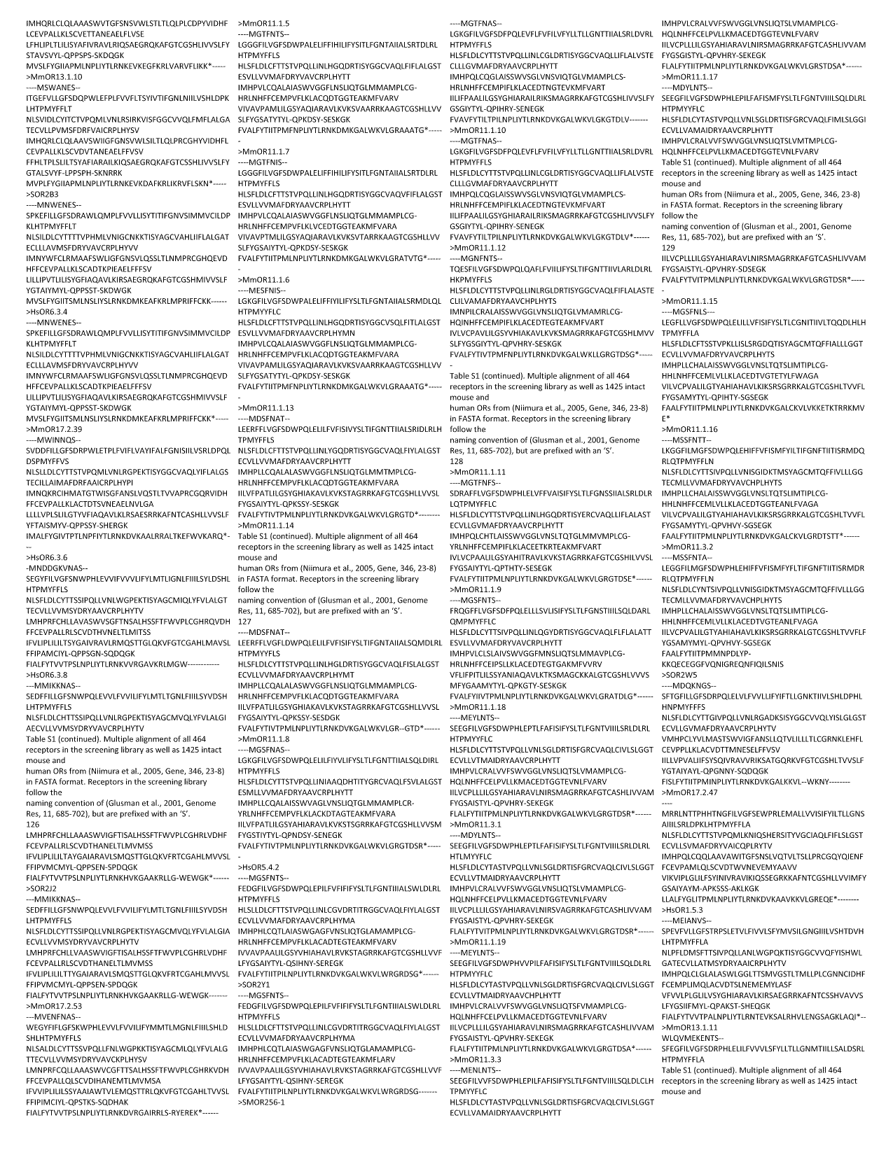

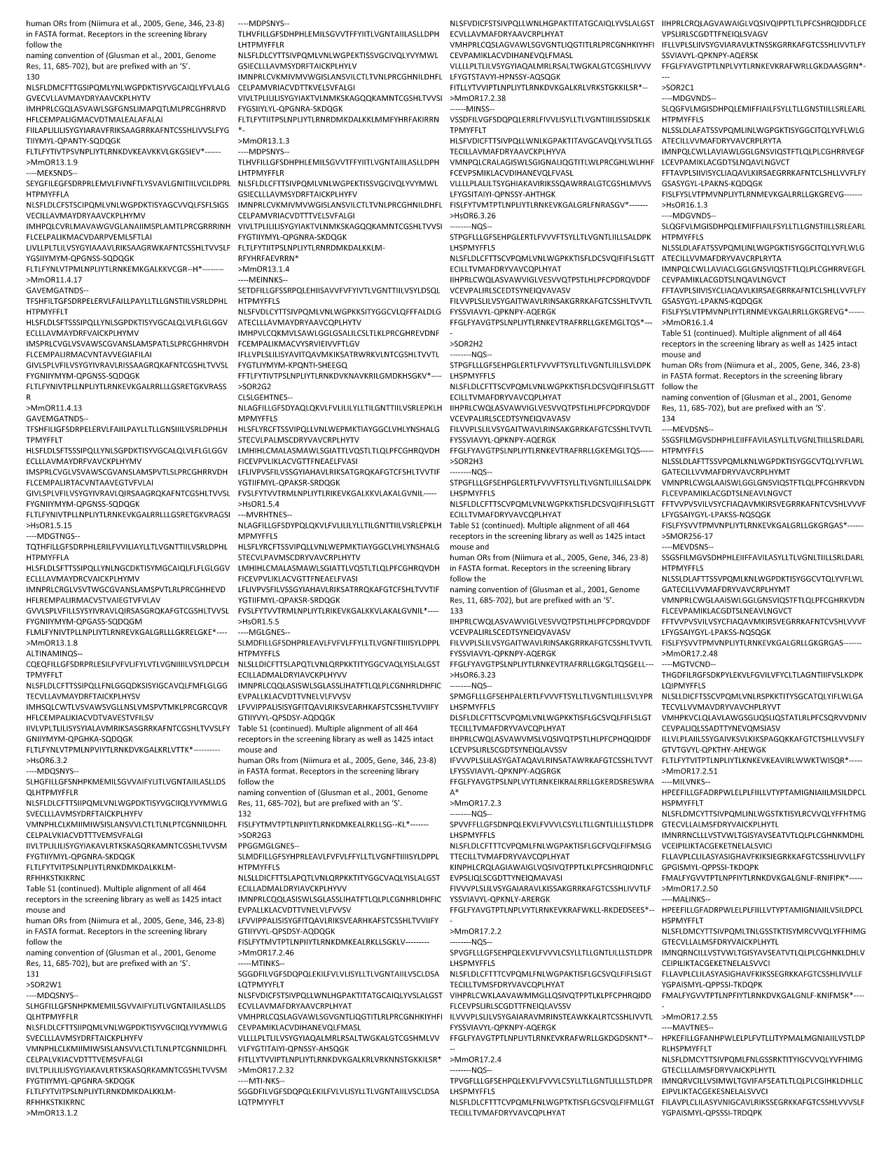

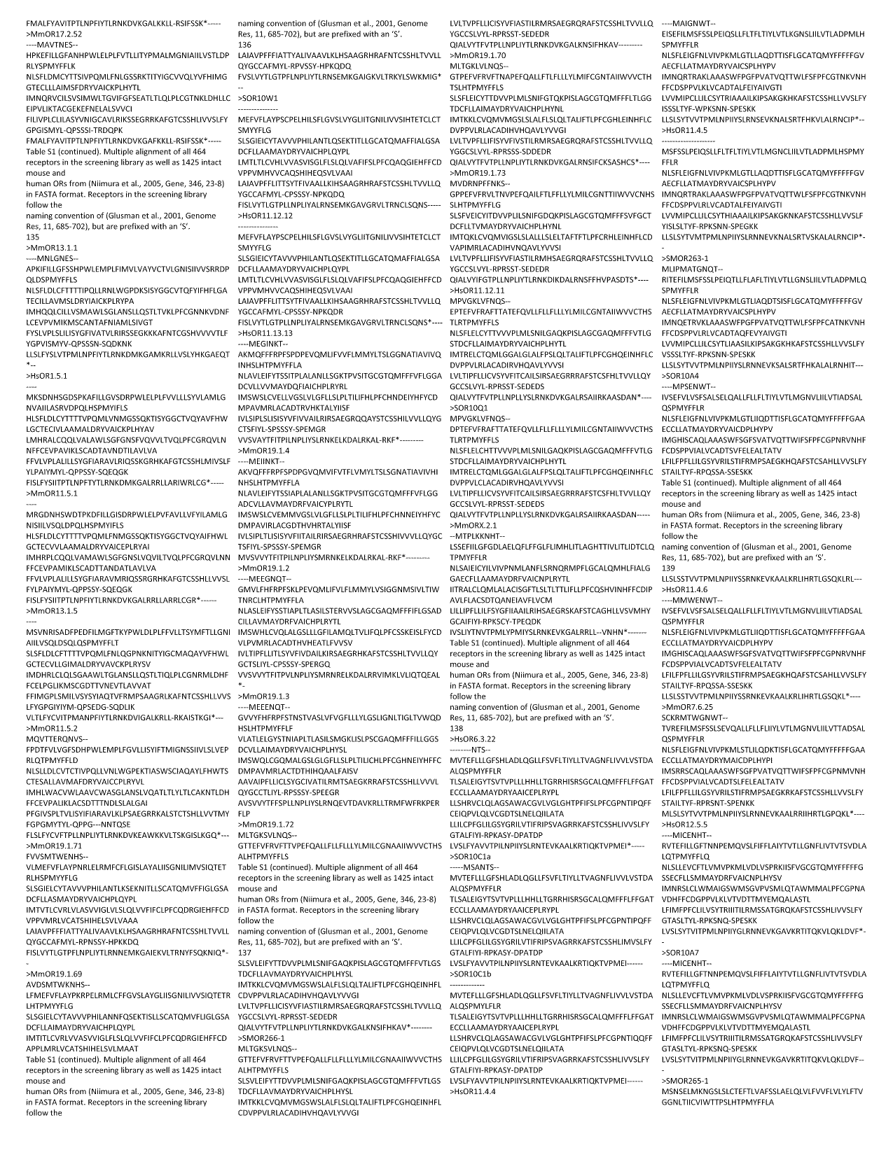

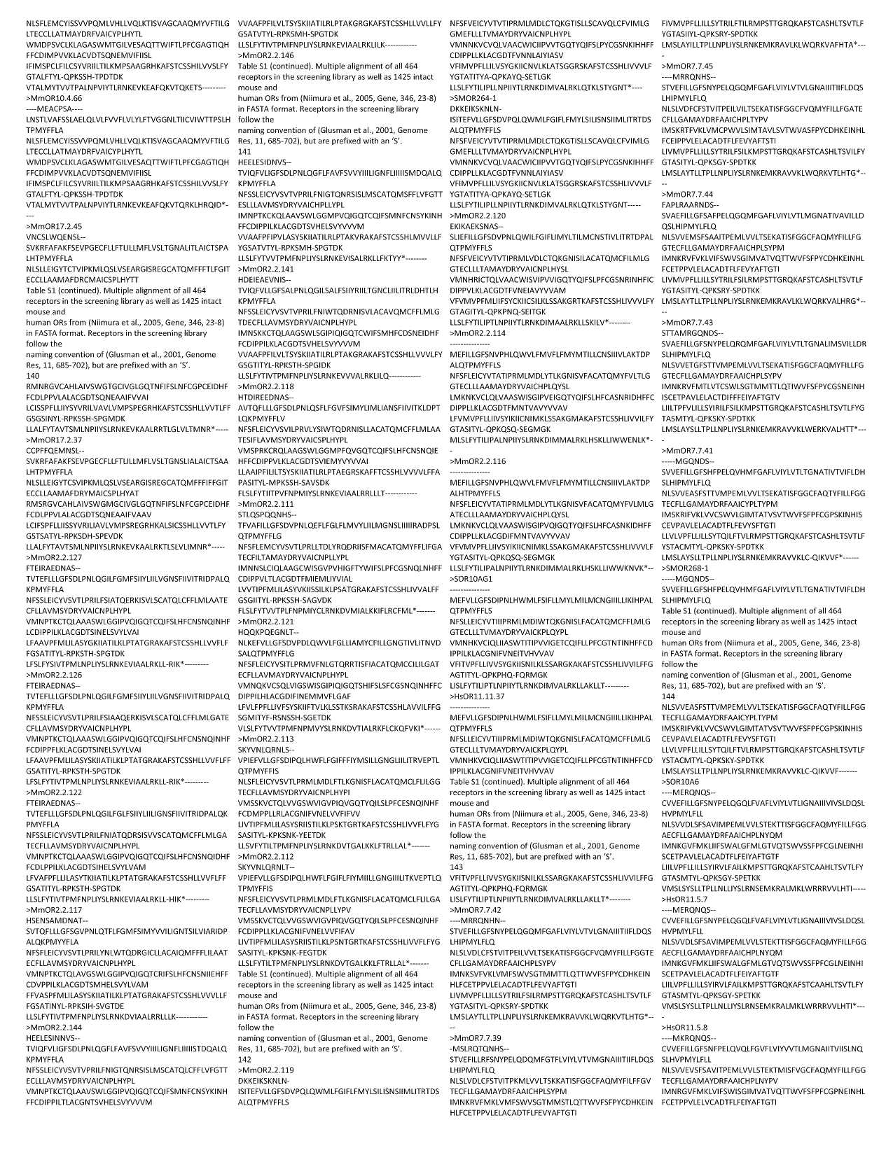

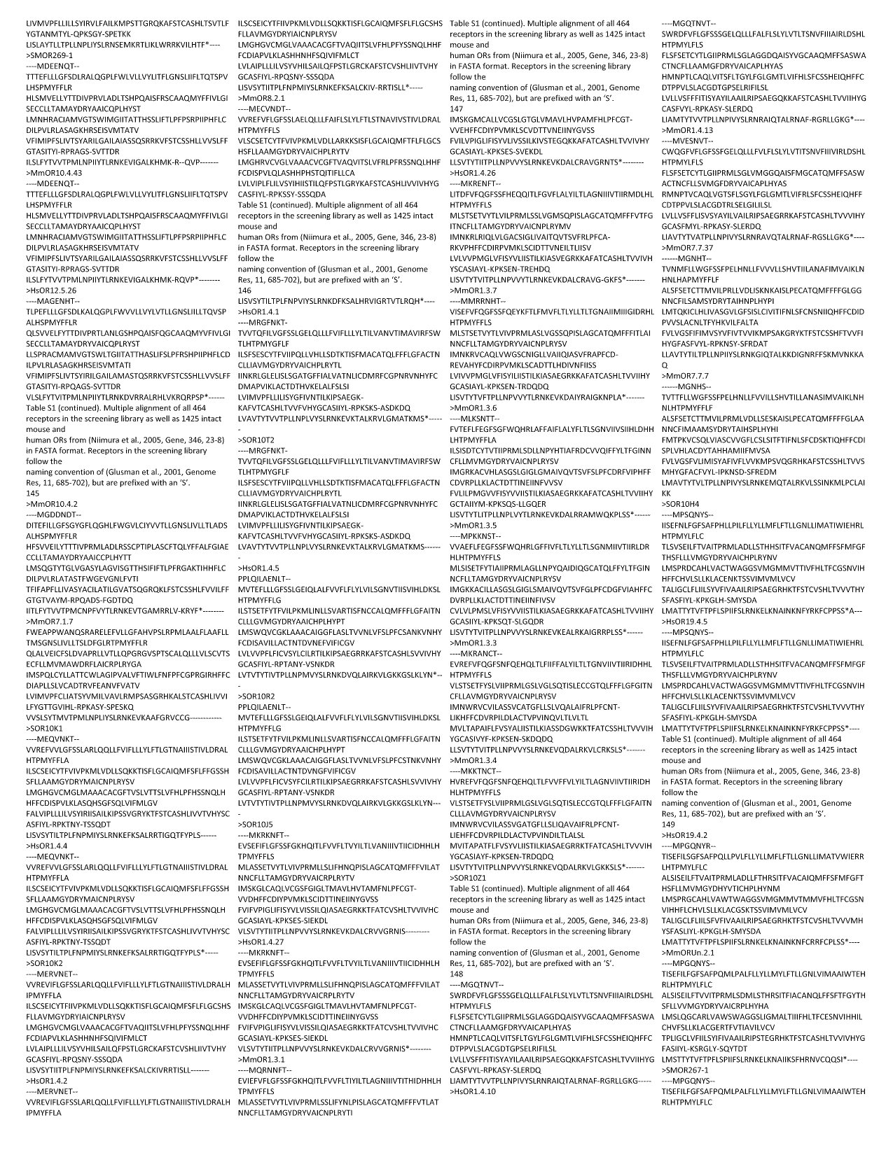

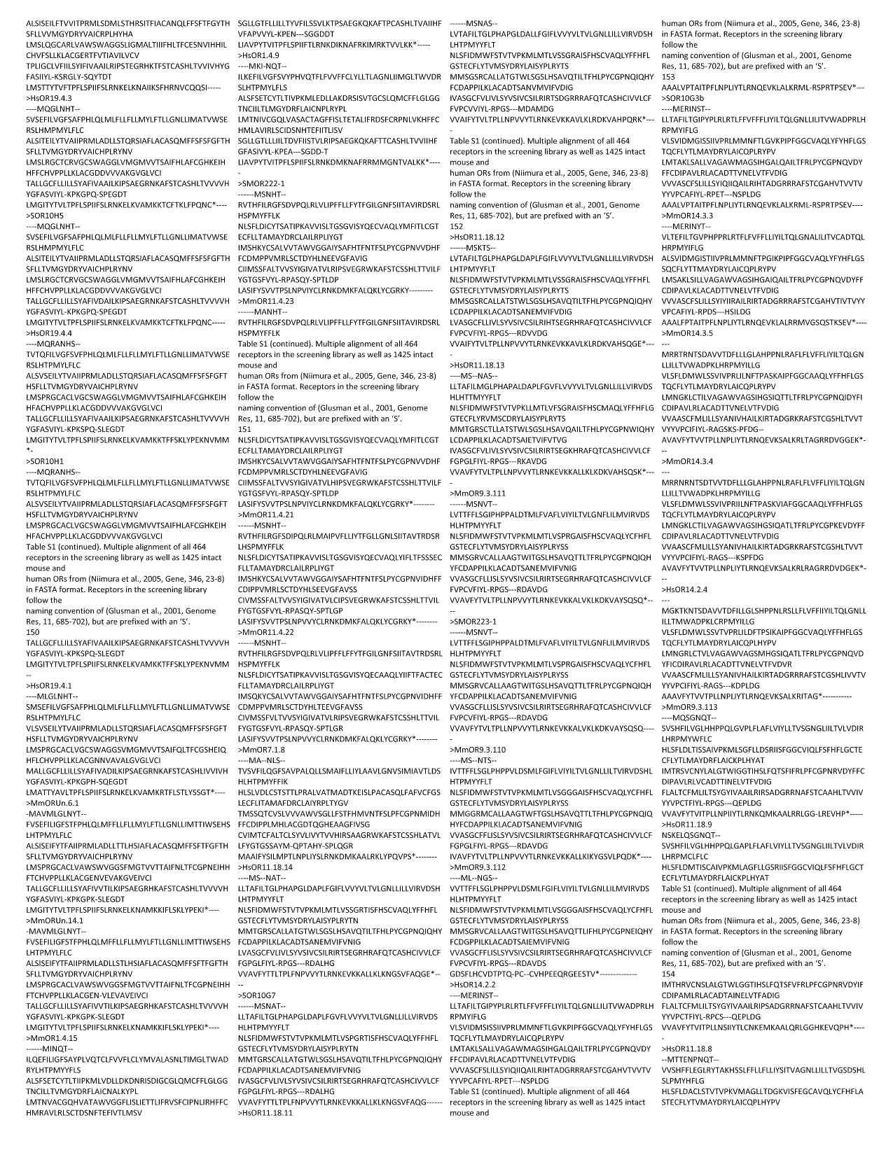

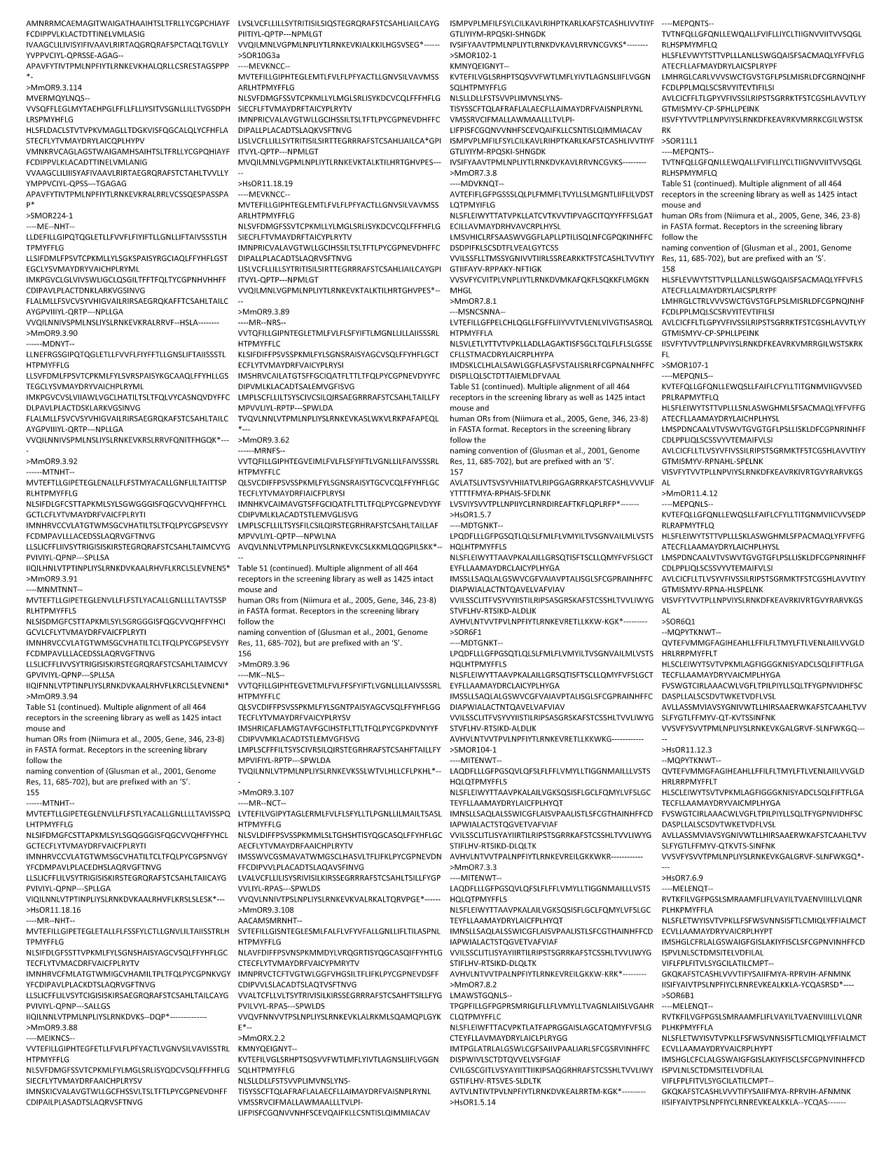

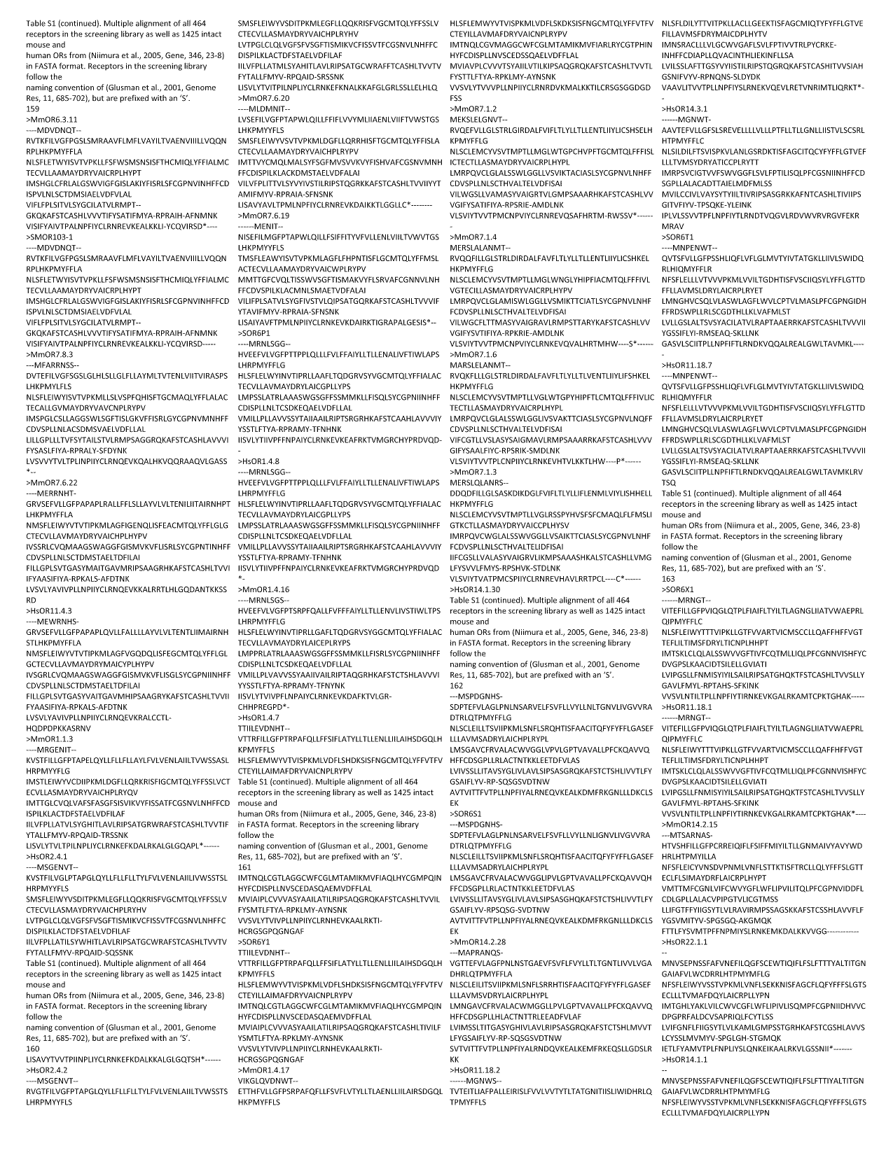

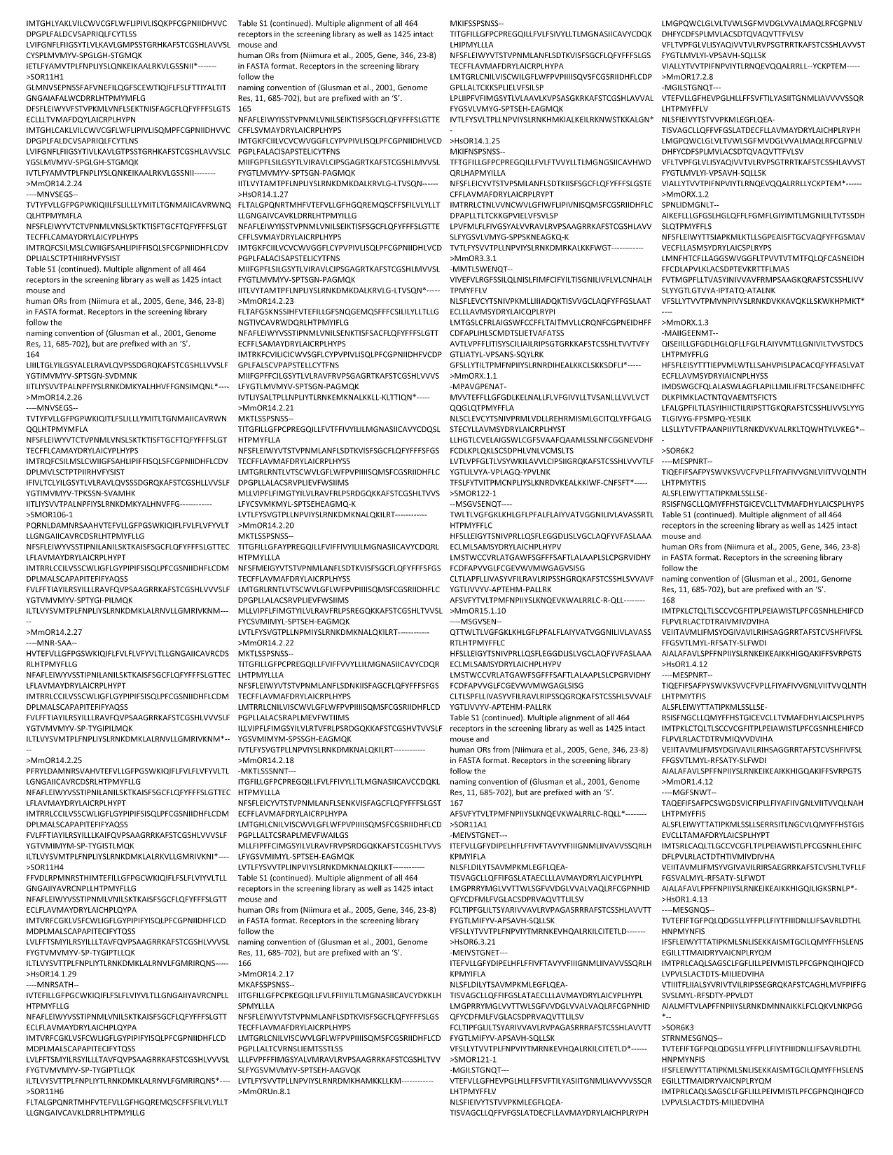

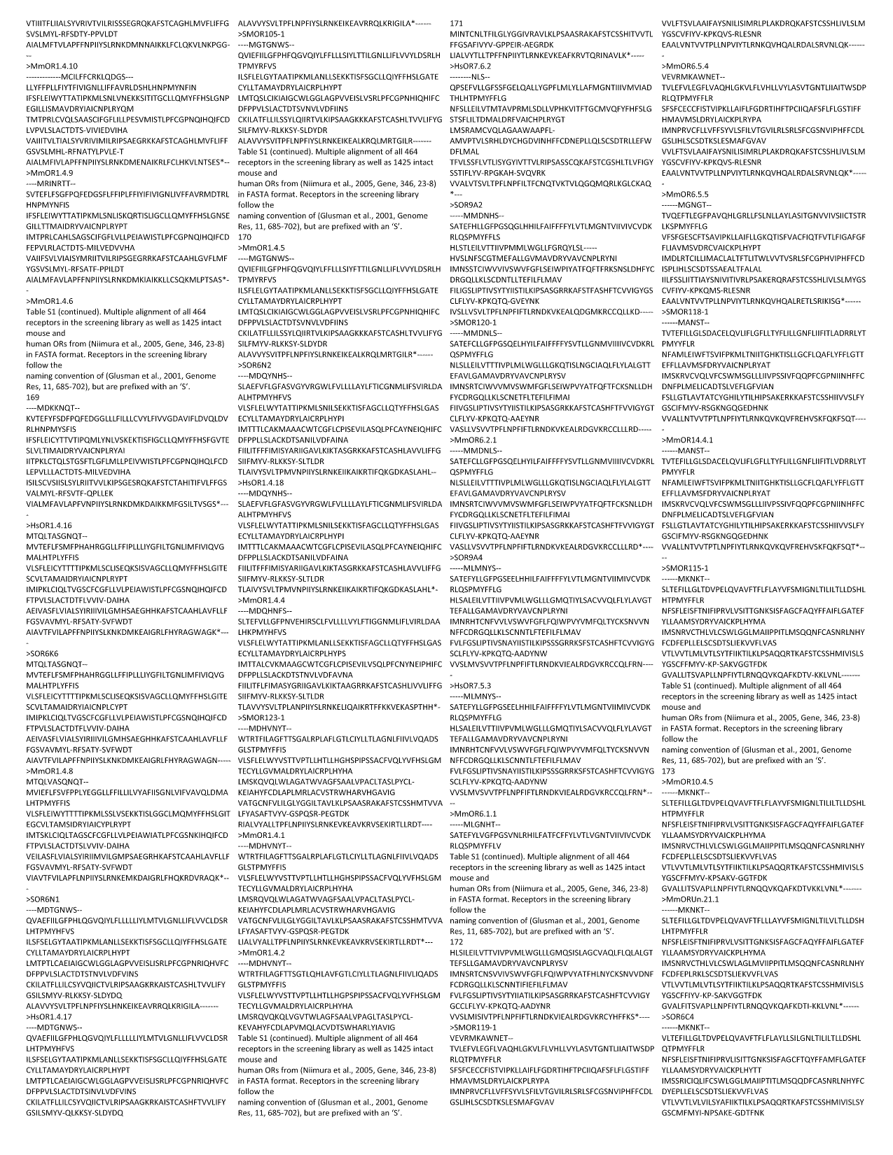

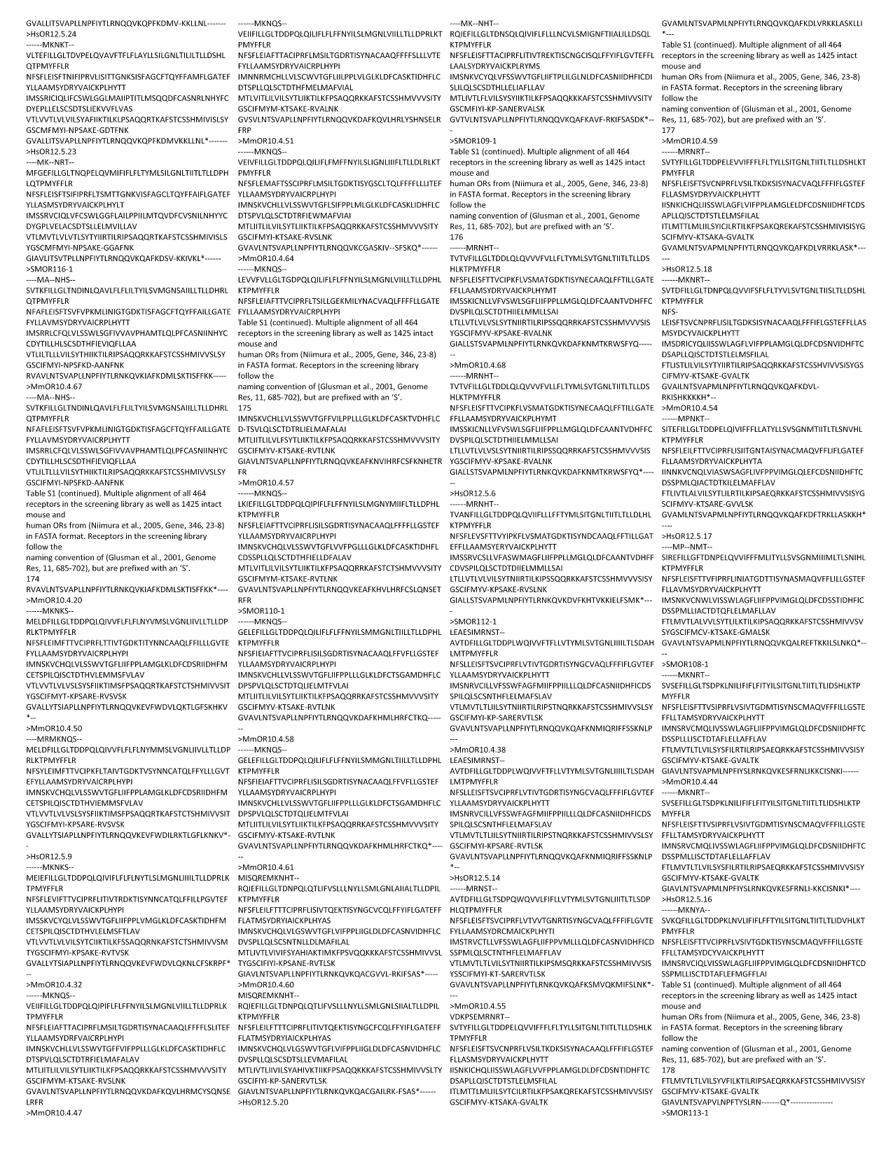

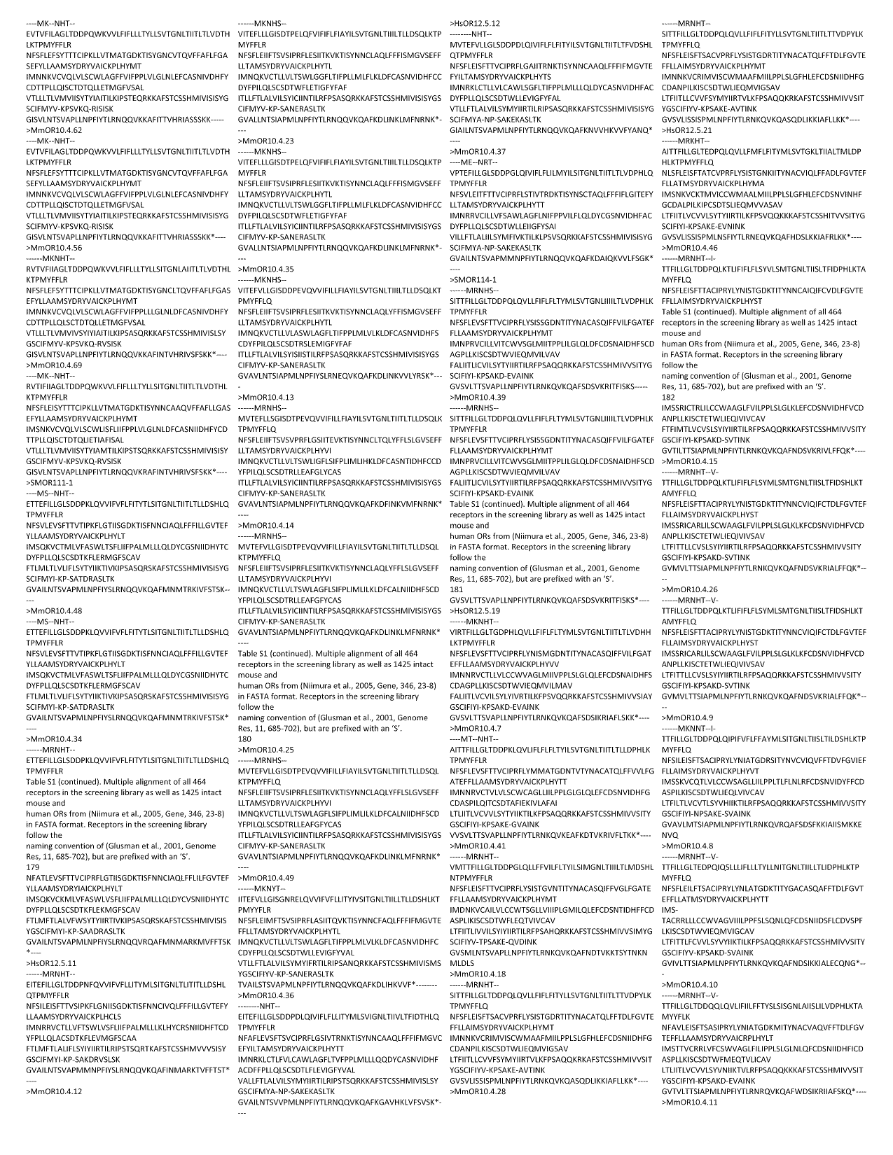

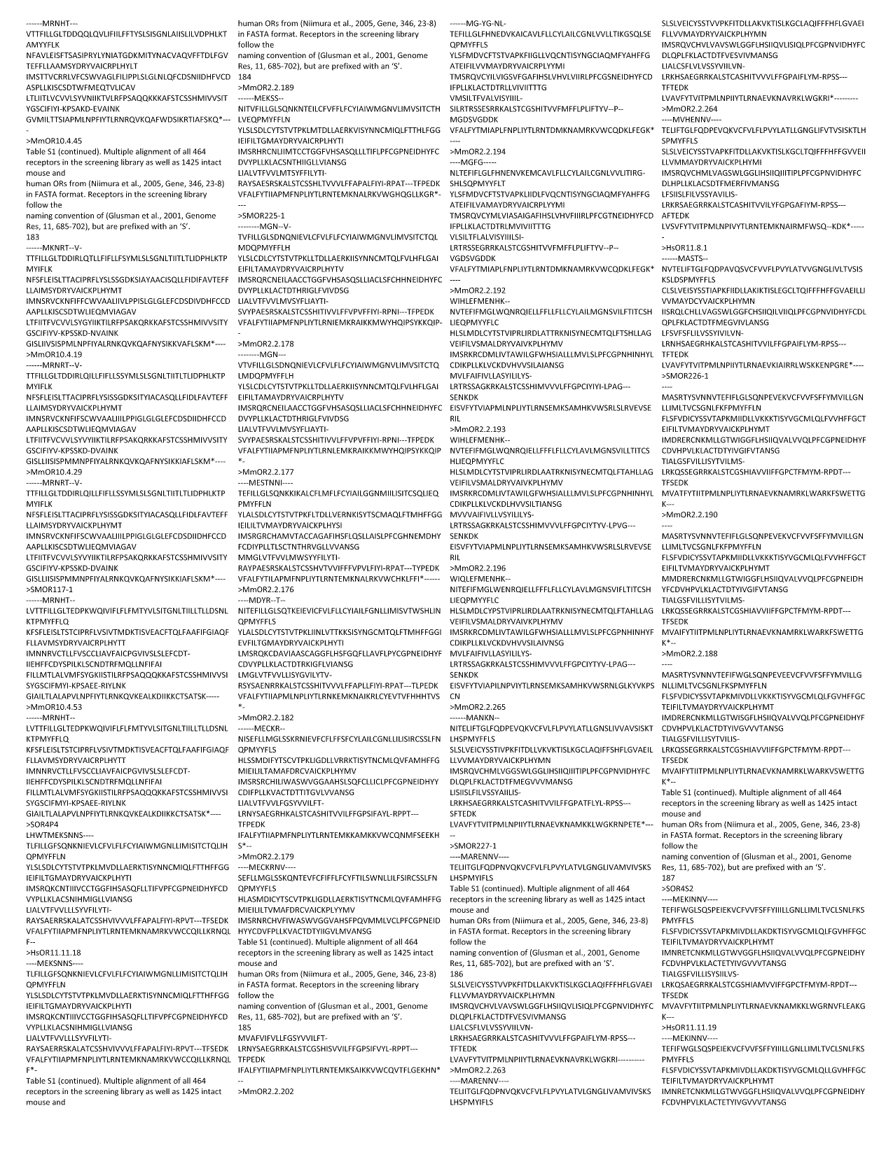

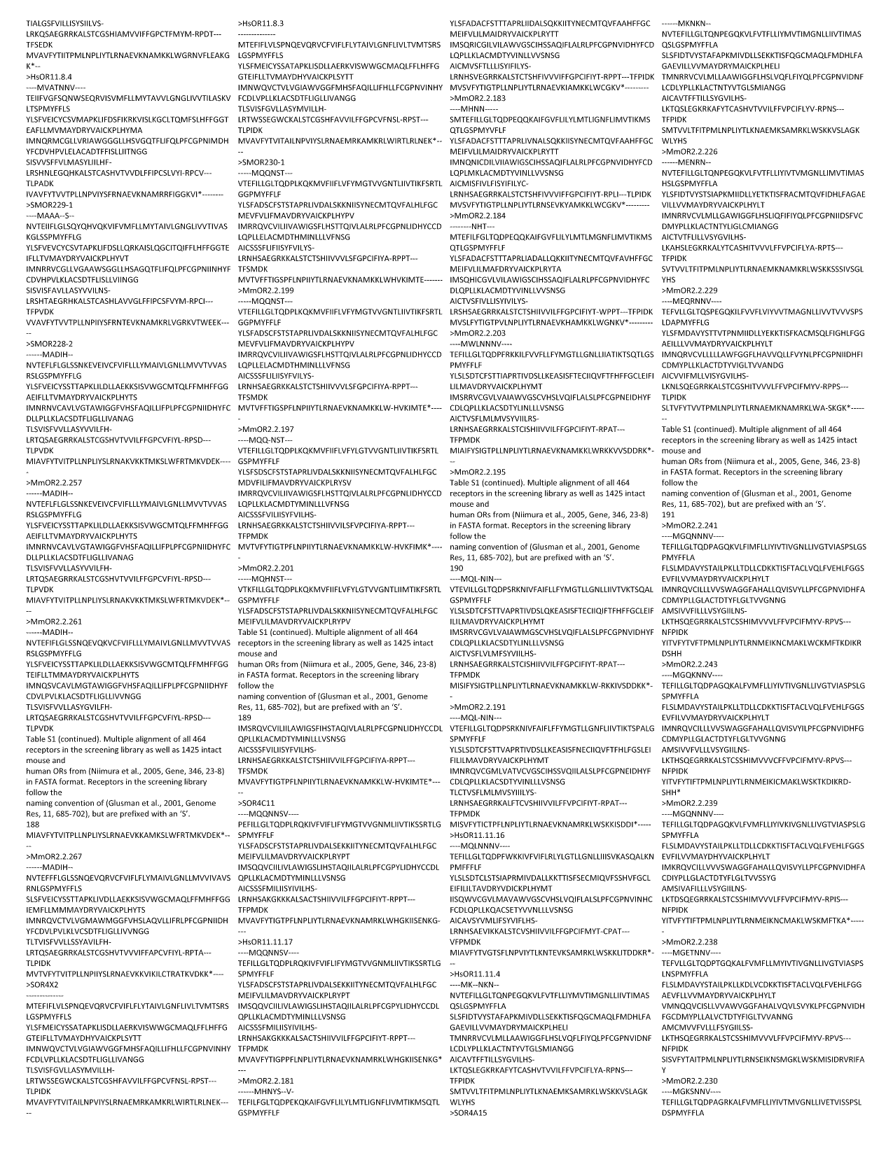

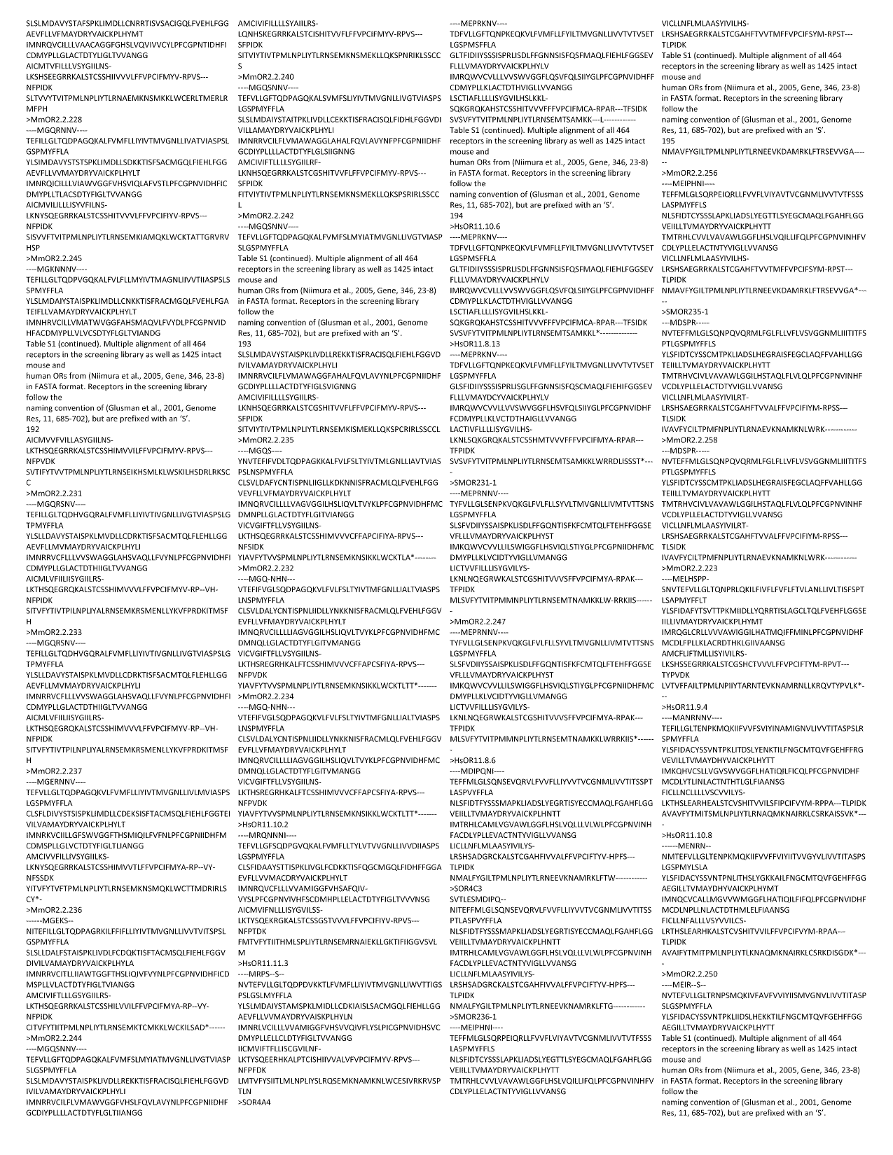

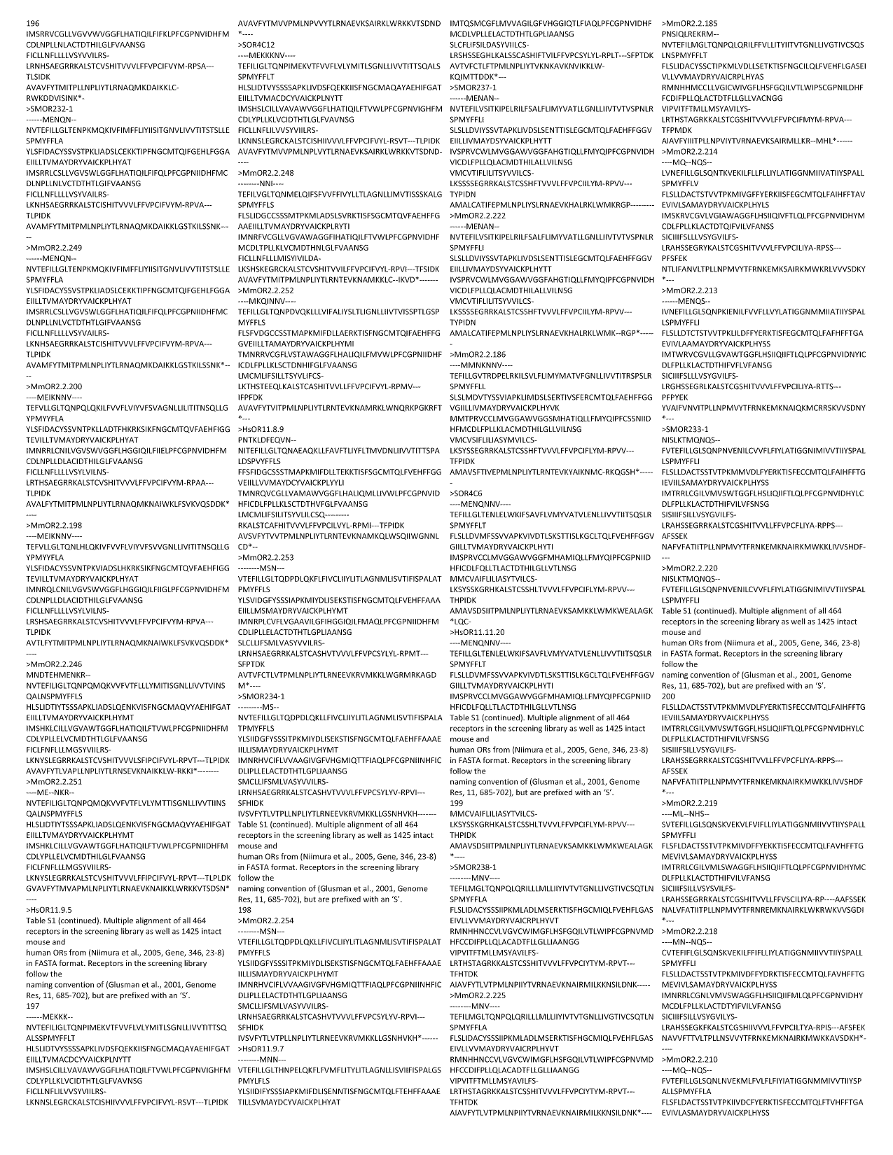

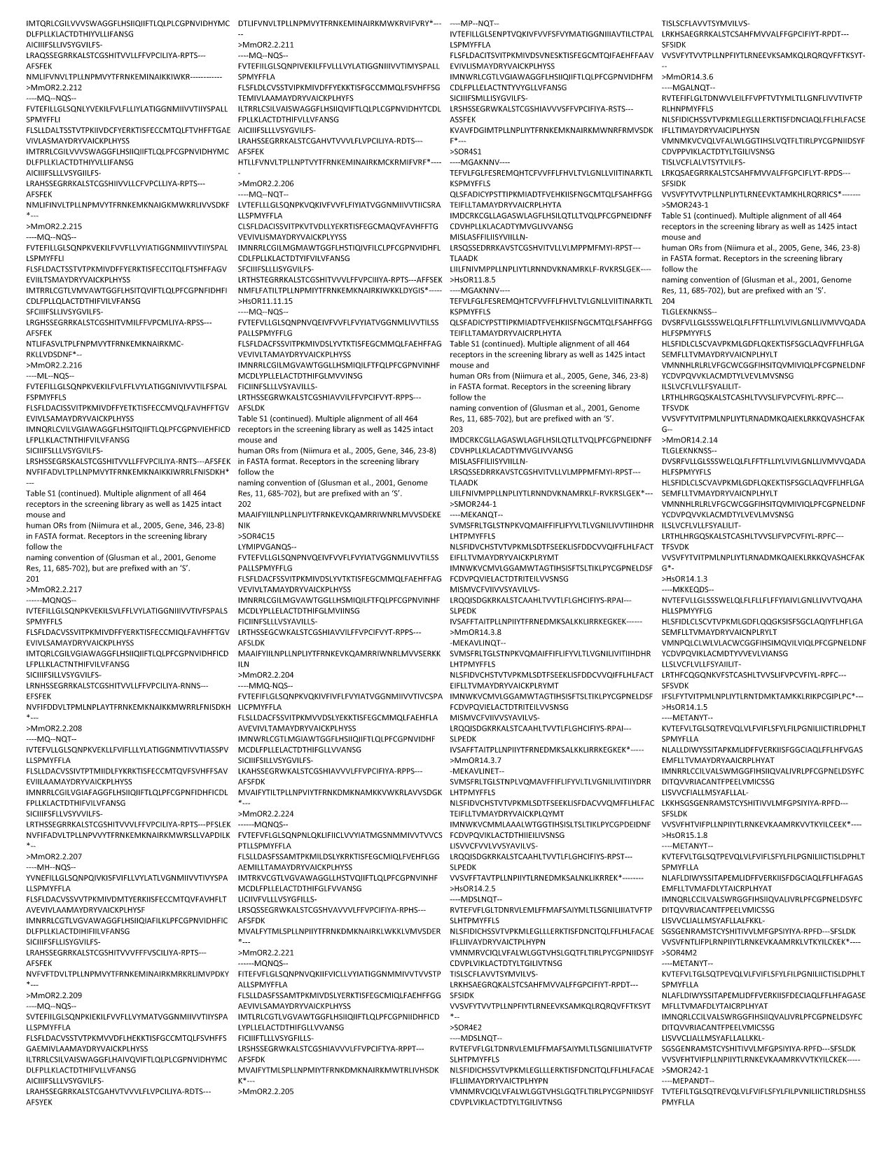

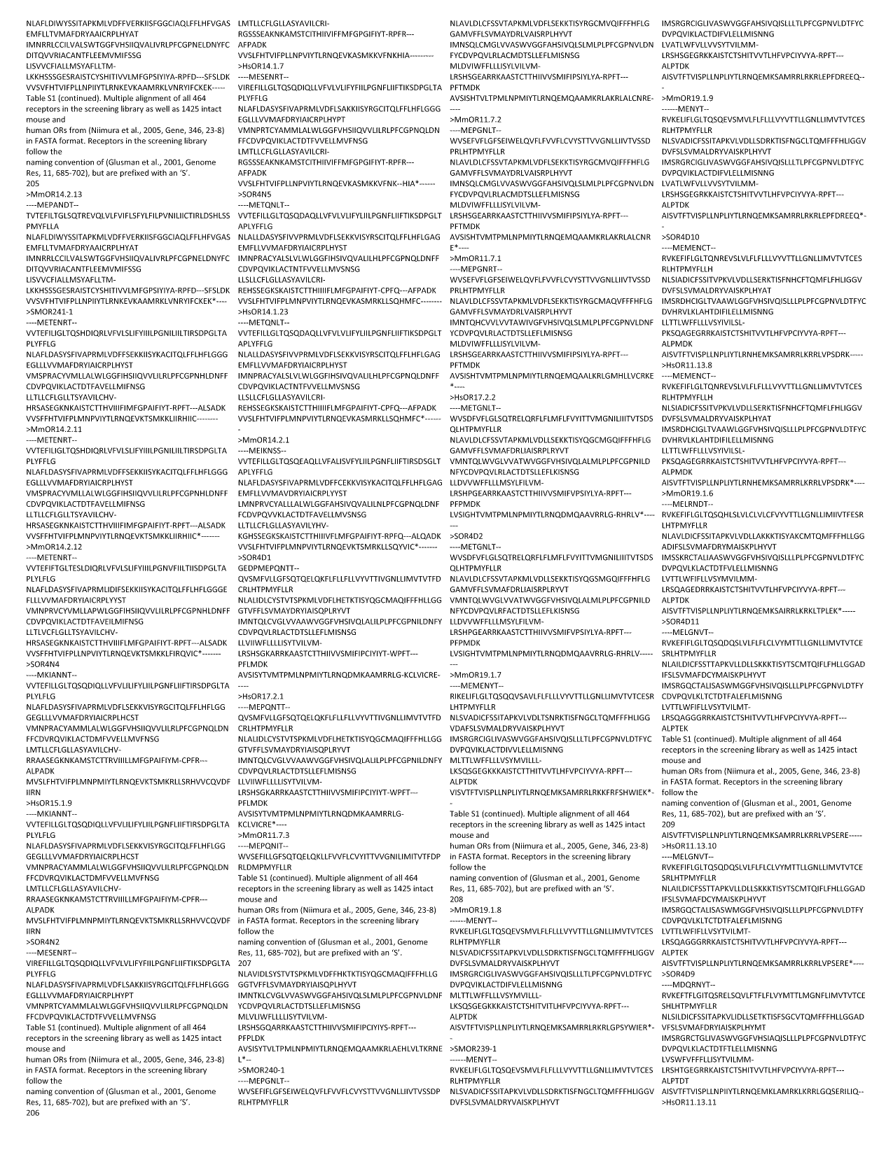

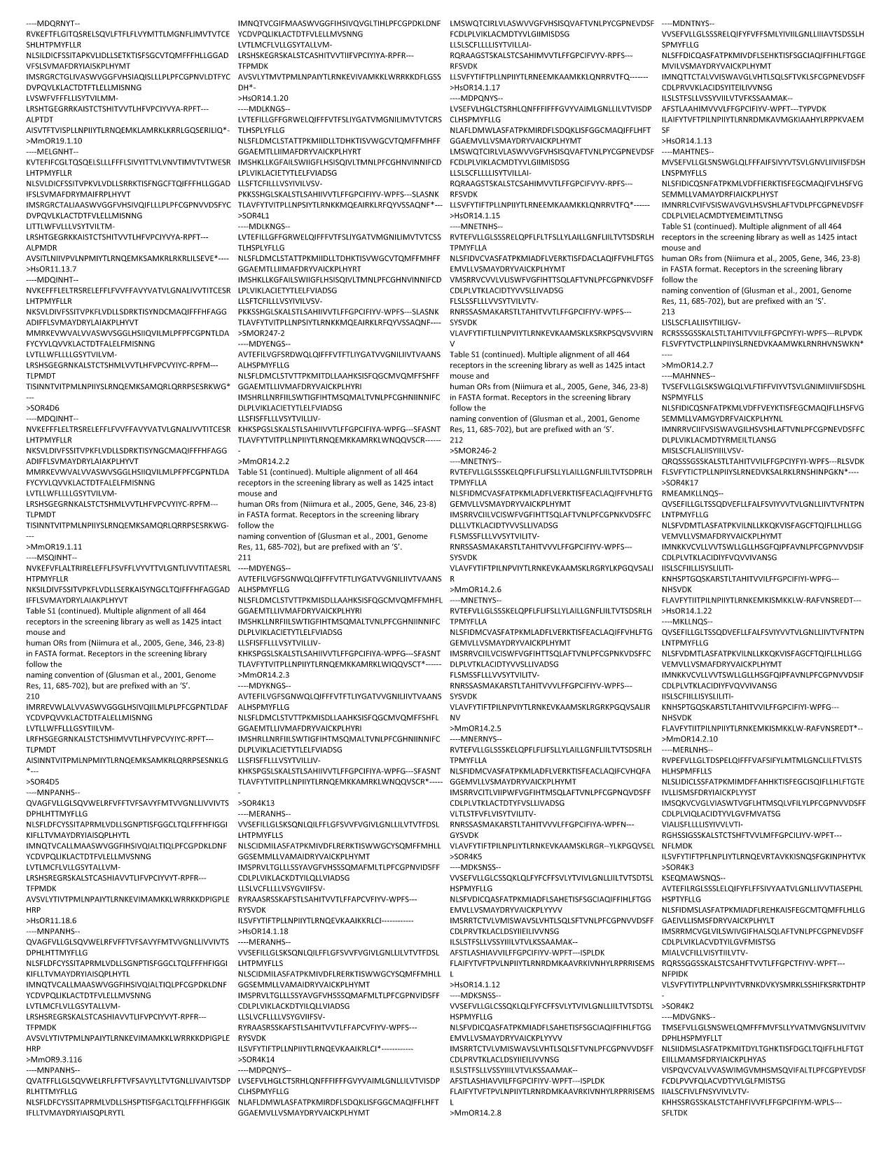

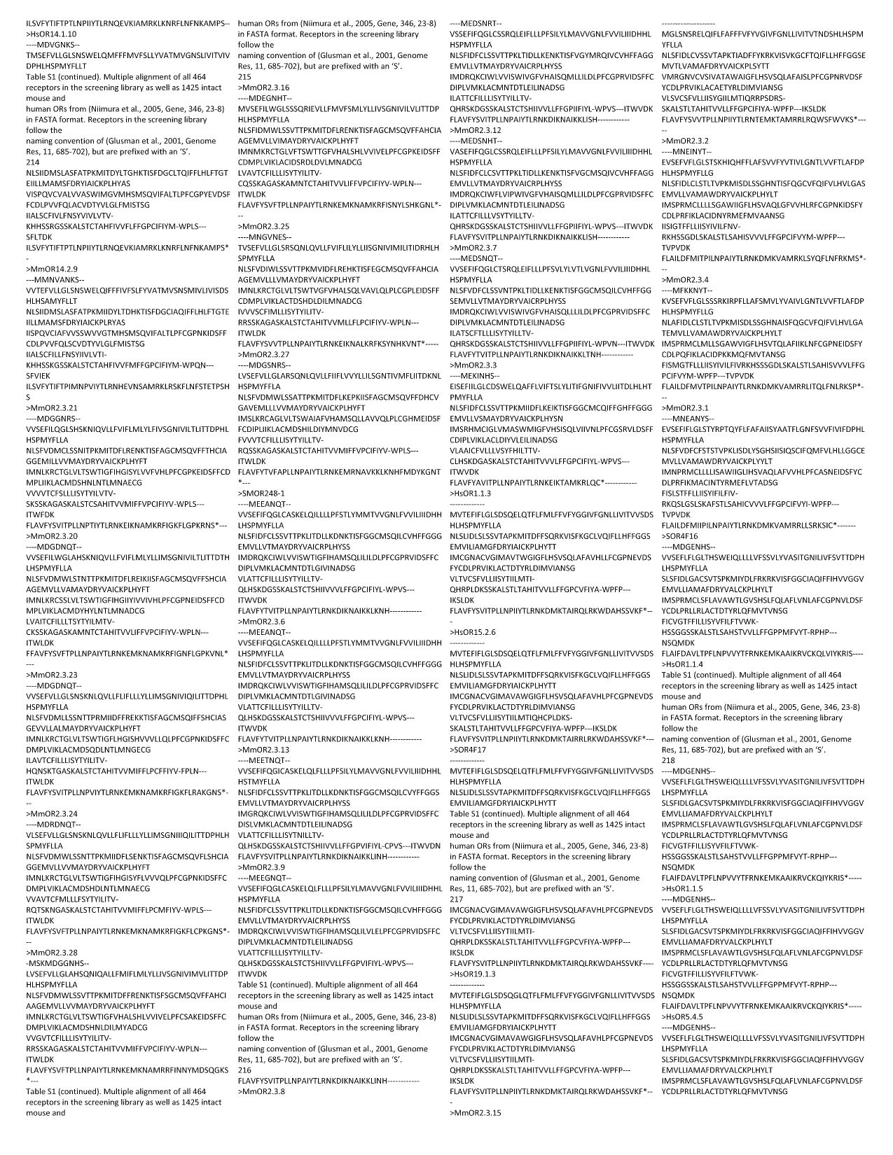

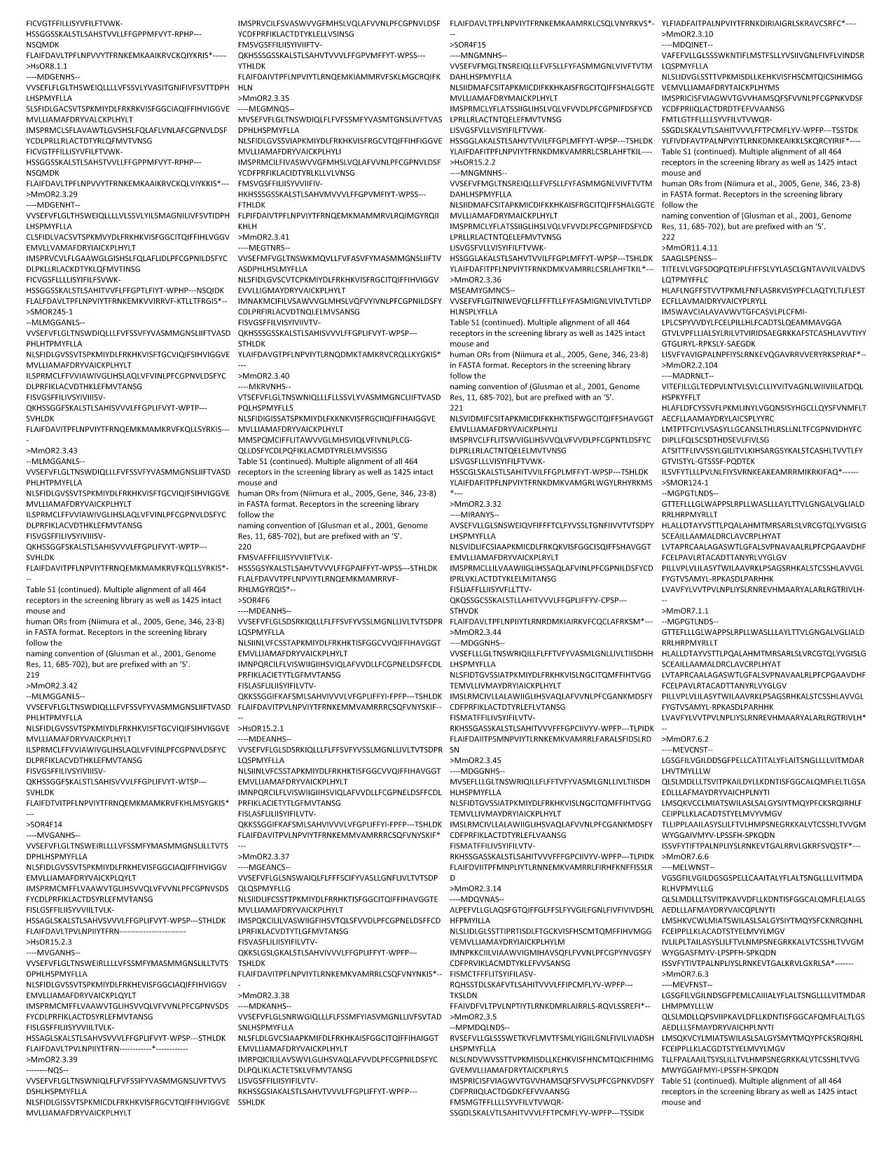

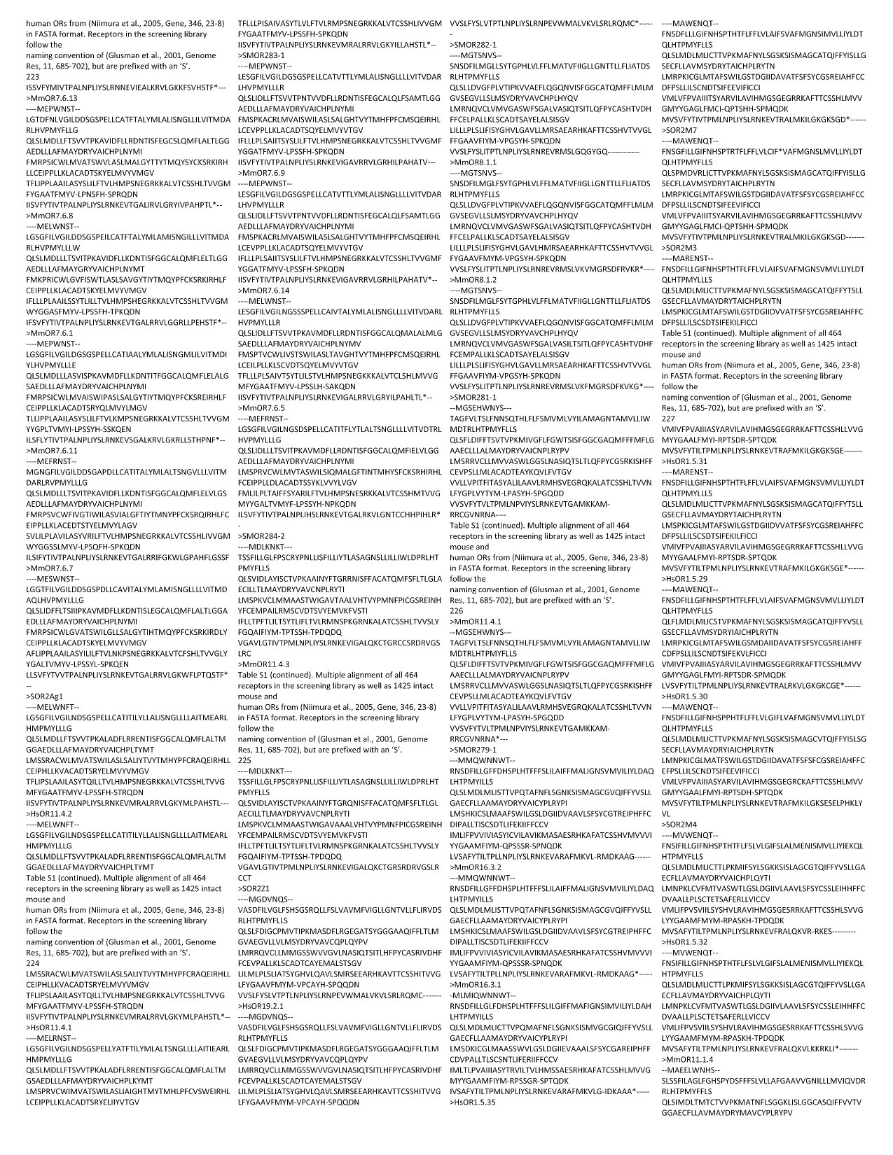

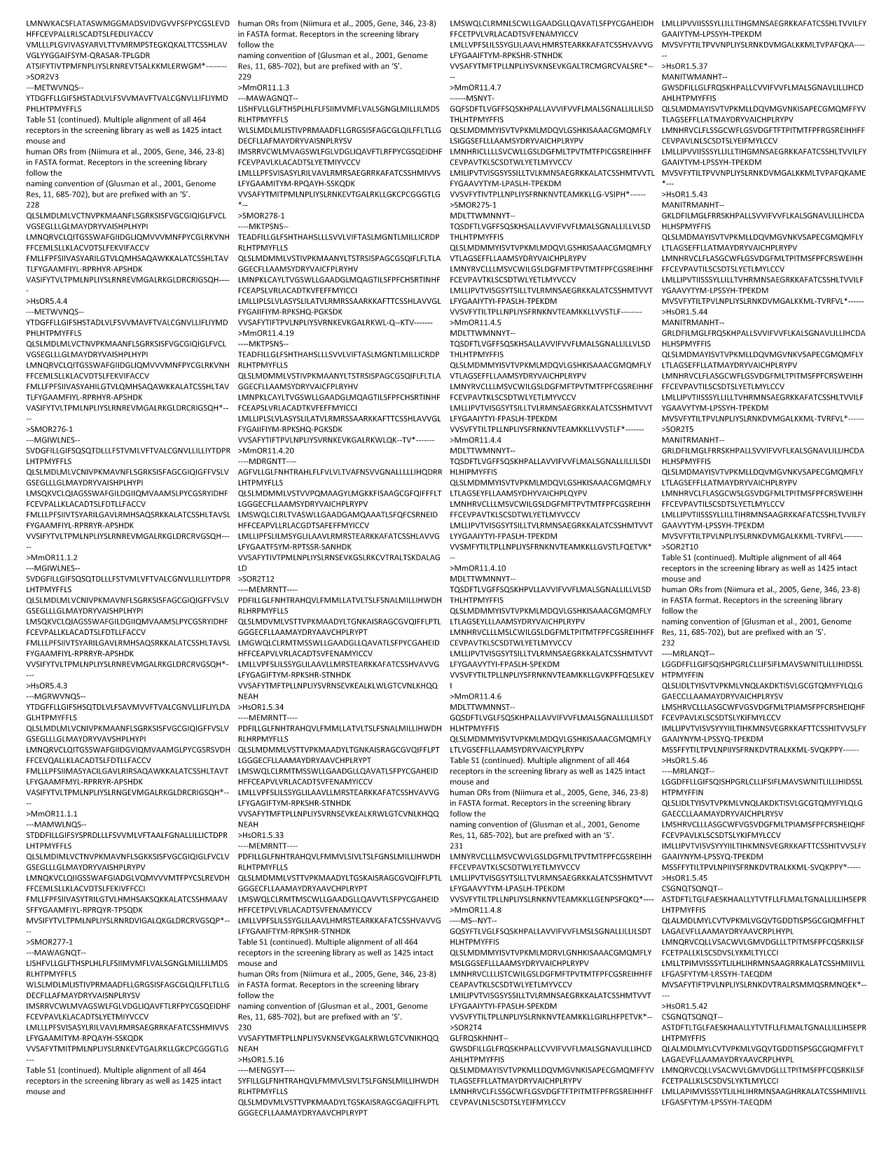

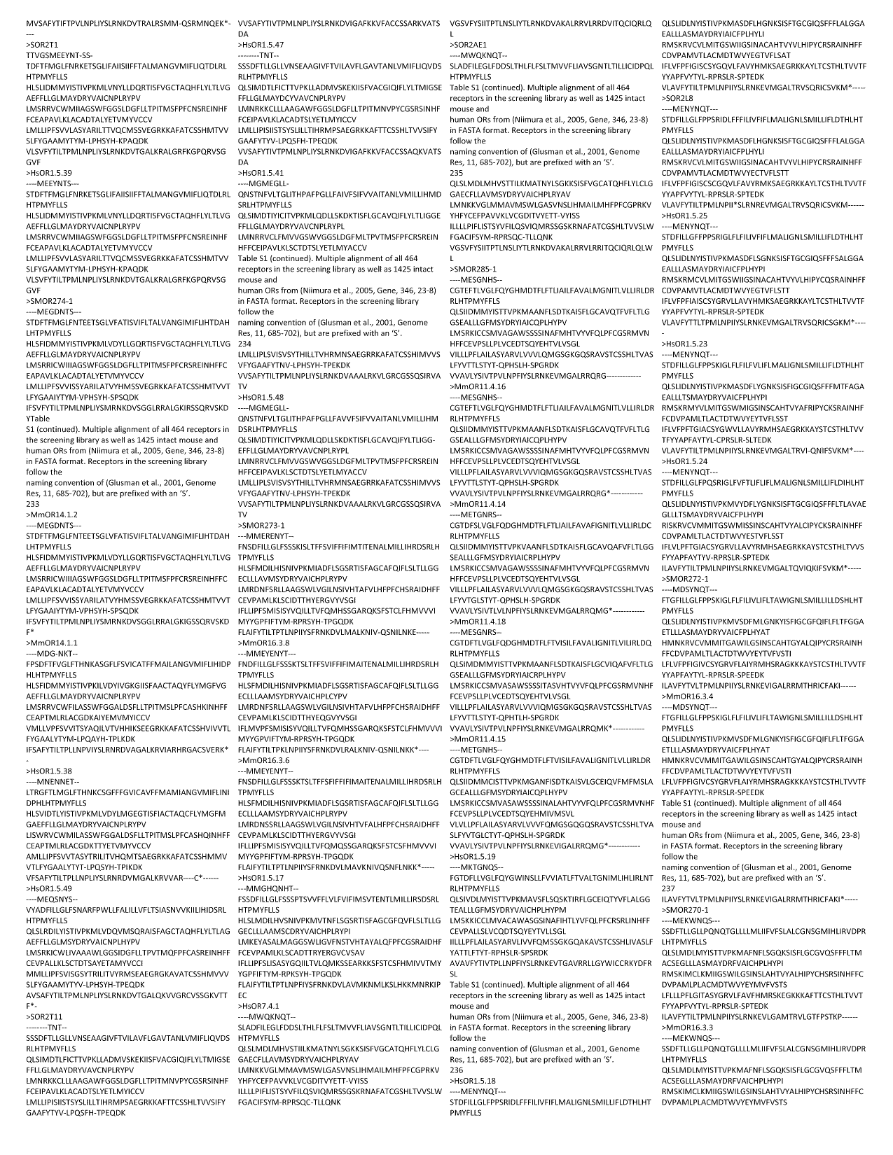

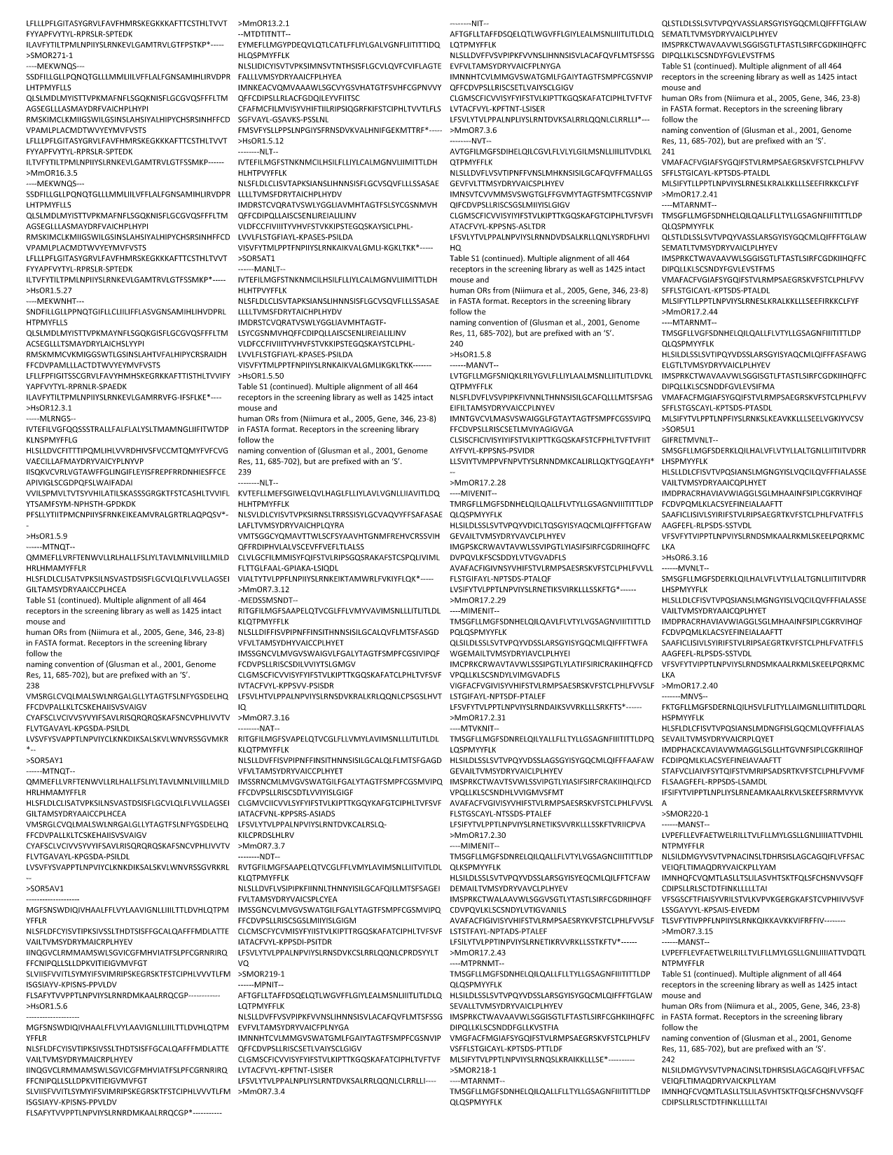

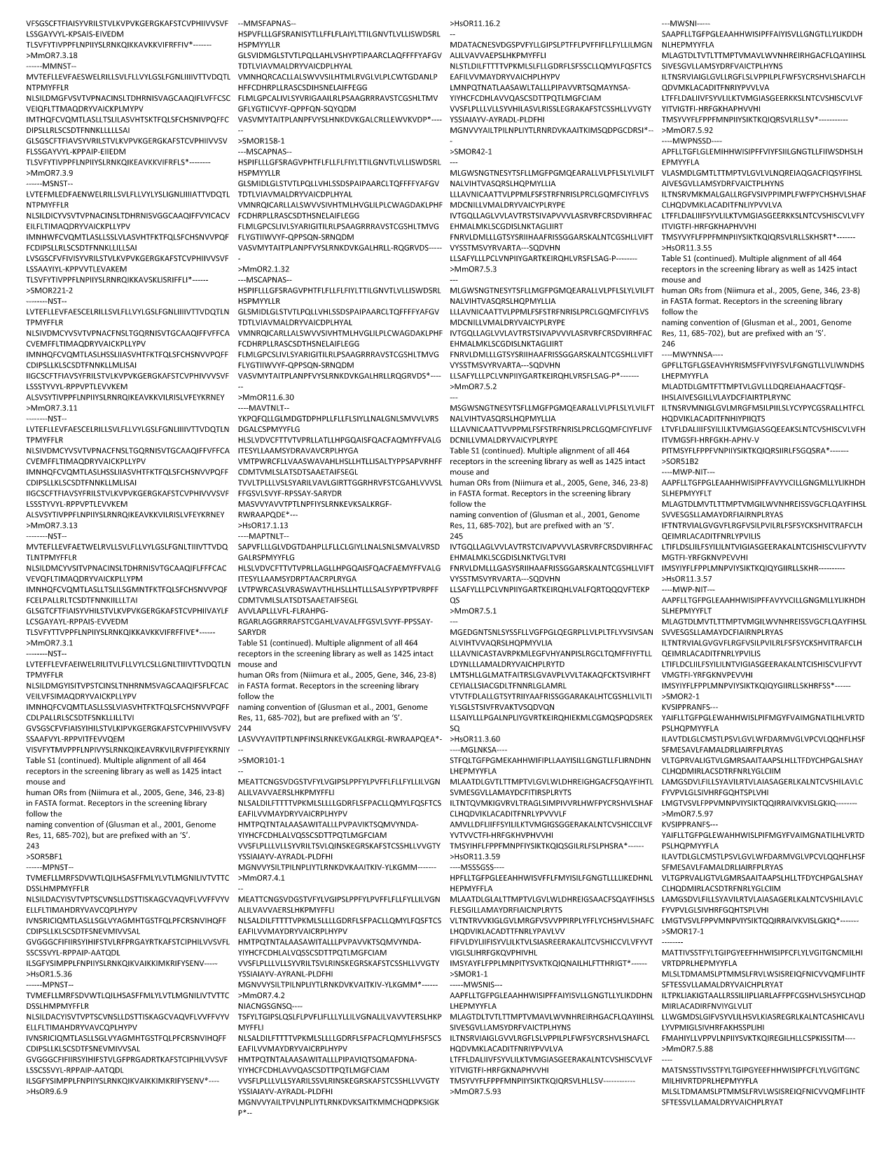

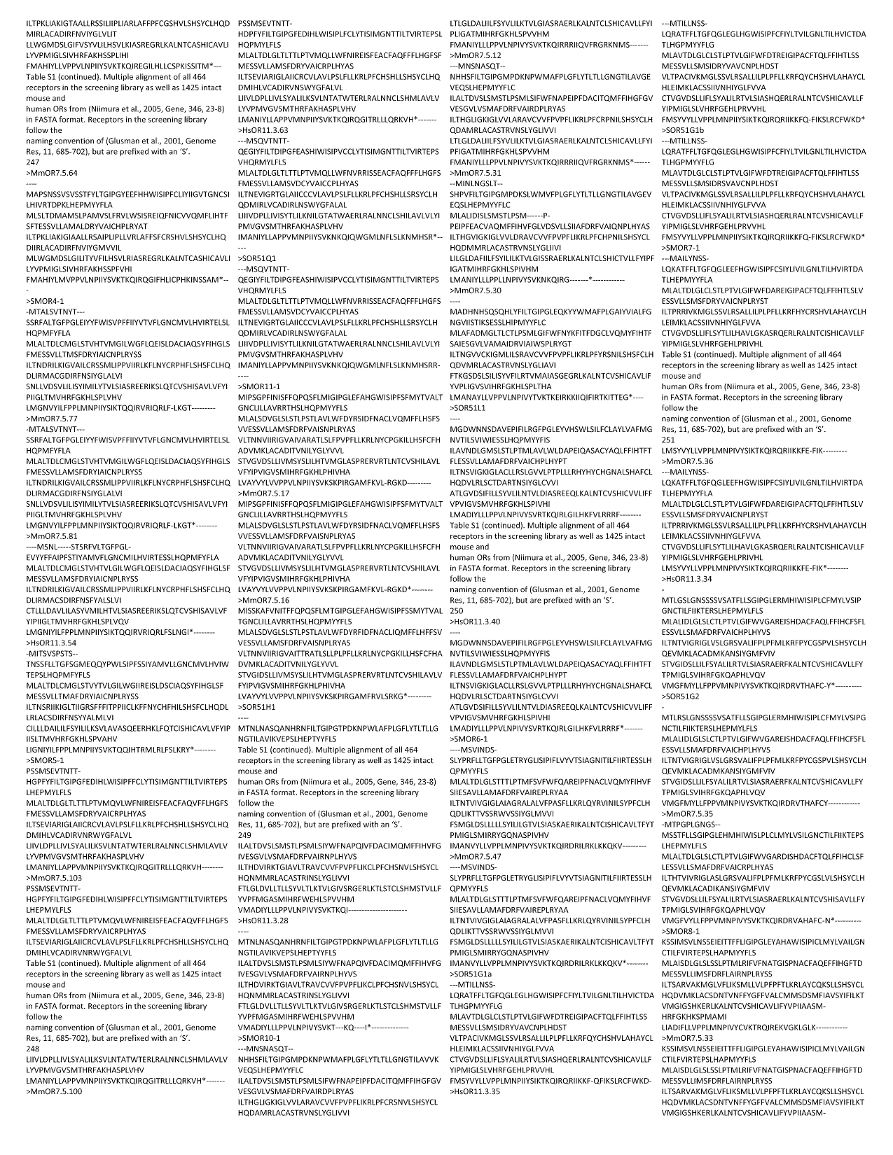

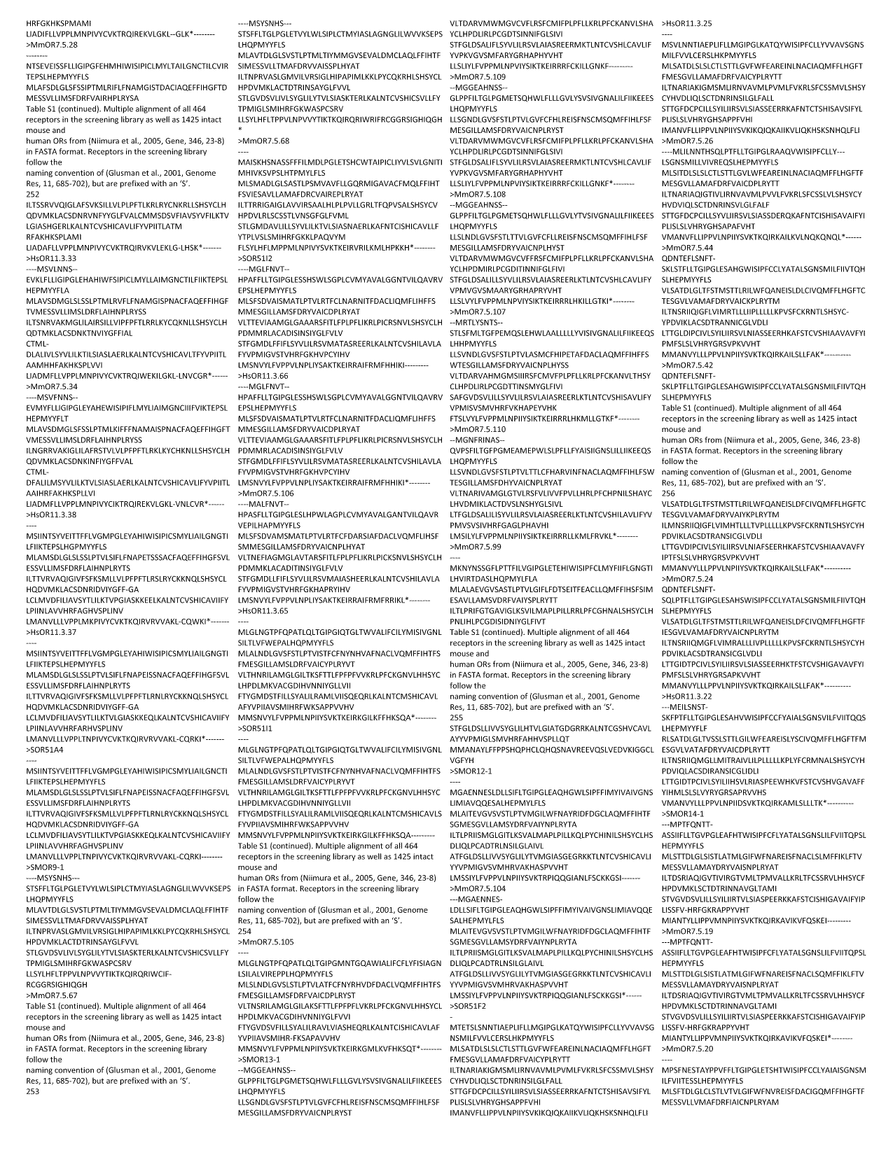

In color vision, three broadly-tuned receptors sense the entire visible range of wavelengths (De Valois et al., Vision Res 33, 1053-65 (1993); herein incorporated by reference in its entirety). In addition, approximately 3,500 narrowly-tuned cochlear hair cells sense the audible spectrum of frequencies. In olfaction, it is not clear if receptors are broadly or narrowly tuned. Given the aforementioned lack of an agreed-upon metric for measuring odorant similarity this question has traditionally been answered in terms of a receptor's number of agonists. These values (number of agonists) are listed for five receptors in FIG. 5A. On the second line of FIG. 5A the sensitivity of the receptor to each odorant is incorporated to create a tuning curve (for sensitivity ordered tuning curves for all receptors see FIG. 14). Note that the x-axis is ordered to place the most sensitive odorant in the center and is different for each receptor. A metric used to define an odorant receptor as “broadly” or “narrowly” tuned should preferable, however, take into account not only the number of agonists to which it responds, but also the similarity of those agonists to each other. On the third line of FIG. 5A the x-axis is ordered to reflect odorant similarity, in other words the x-axis represents the odorant's position along the first principal component of Haddad et al's 32-dimensional odorant space (Haddad et al., Nature Methods 5, 425-429 (2008); herein incorporated by reference in its entirety) (for one-dimensional tuning curves based on the first principal component for all receptors see FIG. 15). This first principal component only describes 19.4% of the variance in odorant space. Using more principal components, as in the fourth line of FIG. 5A, describes more of the variance in odorant space, but beyond three principal components is difficult to display in a figure (for two-dimensional tuning curves based on the first two principal components for all receptors see FIG. 16). The radius of a circle enclosing all of the agonists, as in the fourth line of FIG. 5A, gives a measure of tuning breadth that, unlike the figure, can be scaled to high dimensional space. The radius of a hypersphere enclosing all five receptors' agonists in Haddad et al's 32-dimensional odorant space is listed on the fifth line of FIG. 5A, and for all receptors in FIG. 5B. The results reveal that the mammalian ORs vary along a continuum of tuning breadths. In other words, some receptors are broadly tuned, responding to a large number of odorants that occupy a large area of odorant space (i.e. are structurally dissimilar), others are more narrowly tuned, and some respond to only a small number of closely related odorants.

### Receptor-Response to Enantiomers

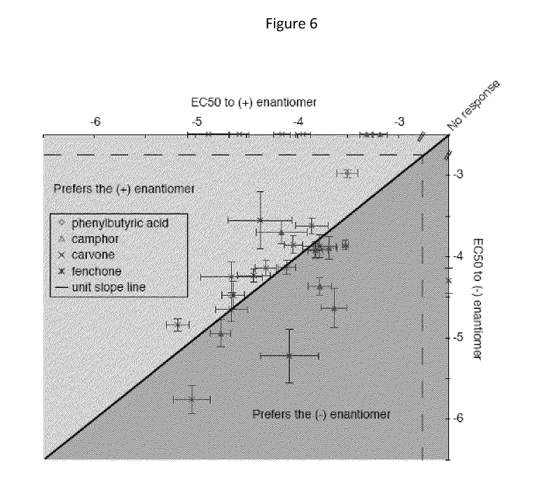

Enantiomers—stereoisomers that are nonsuperimposable mirror images of each other—impose a constraint on olfactory theories because, even though they have many identical properties, mammals can discriminate between the members of some pairs (Laska et al., Neuroscience 144, 295-301 (2007); Rubin et al., Nat Neurosci 4, 355-6 (2001); Joshi et al., Chemical Senses 31, 655-64 (2006); Linster et al., J Neurosci 22, 6842-5 (2002); each herein incorporated by reference in their entirety). Four pairs of enantiomers were examined: phenylbutyric acid, carvone, fenchone, and camphor. The (+) and (−) enantiomers of carvone activate overlapping but distinct sets of olfactory neurons in mice (Ma et al., PNAS, 97:12869 (2000); herein incorporated by reference in its entirety). Consistent with these previous reports, it was found that the response of an OR to one enantiomer is highly correlated with its response to the other enantiomer (r=0.85, p<0.0001). A receptor basis for the ability to perceptually distinguish between three of the four enantiomers was found: for example MOR107-1 is activated only by the (−) enantiomer of fenchone and MOR271-1 is only activated by the (+) enantiomer of fenchone. At some doses, MOR2-1 responded more strongly to the (+) enantiomer of 2-phenylbutyric acid, no receptors responded to only one enantiomer of 2-phenylbutyric acid (FIG. 6). Enantiomers were the most similar odorant pairs in terms of physicochemical descriptors (FIGS. 3B,D).

### Functional Comparison of Class I and Class II Odorant Receptors

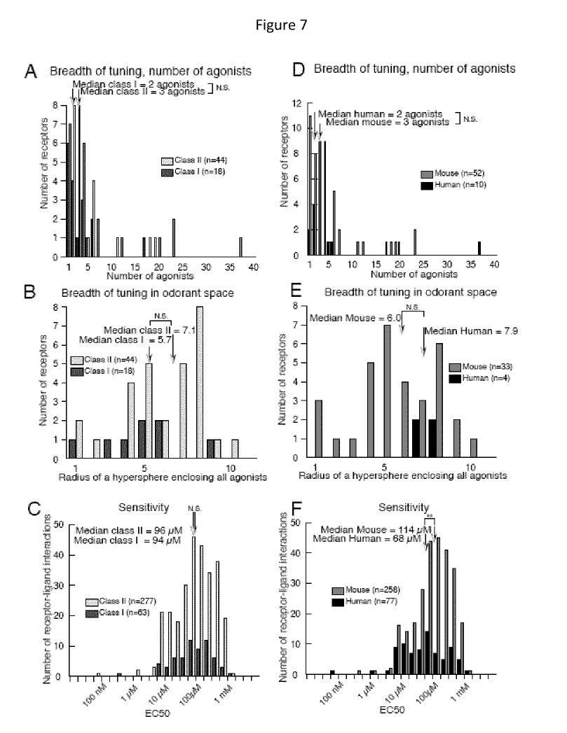

Class I and class II ORs did not differ in number of agonists (p=0.11, Mann-Witney U-test) (FIG. 7A), breadth of odor tuning in odorant space (p=0.21, Mann-Witney Utest) (FIG. 7B), or sensitivity to odorant concentration (p=0.99, Mann-Whitney U-test) (FIG. 7C). However, it was possible to differentiate class I OR agonists from class II OR agonists using a machine-learning algorithm trained on the physicochemical descriptors of the agonists (d′=1.98, p<0.0001).

It was then asked what molecular features best differentiated class I from class II agonists. The top 10 descriptors are shown in Table IA. Agonists for class I ORs are significantly more hydrophilic than agonists for class II ORs (median class I hydrophilic factor=−0.2440, mean class II hydrophilic factor=−0.8020, p<0.001, Mann-Whitney U-test after Bonferroni correction for 1664 descriptors), and have a higher topological polar surface area (median class I TPSA=37.3, mean class II TPSA=17.1, p<0.001, Mann-Whitney U-test after Bonferroni correction for 1664 descriptors).

### Functional Comparison of Human and Mouse Receptors

Humans have fewer than 400 ORs with an intact open reading frame whereas mice have more than 1000 (Zhang et al., Nat Neurosci 5, 124-33 (2002); Glusman et al., Genome Res 11, 685-702 (2001); Niimura et al., Proc Natl Acad Sci USA 100, 12235-40 (2003); Malnic et al., Proc Natl Acad Sci USA 101, 2584-9 (2004); Godfrey et al., Proc Natl Acad Sci USA 101, 2156-61 (2004); each herein incorporated by reference in their entirety). The present invention is not limited to a particular mechanism. Indeed, an understanding of the mechanism is not necessary to practice the present invention. Nonetheless, one hypothesis to account for this difference in OR number is that humans may have preferentially retained broadly-tuned receptors to retain the ability to detect most odorants at the expense of sensitivity to low concentration odorants (Lapidot et al., Genomics 71, 296-306 (2001); herein incorporated by reference in its entirety). There was not a significant difference between mouse and human receptors in the number of agonists (p=0.25, Mann-Whitney U-test) (FIG. 7D) or breadth of tuning in odorant space (p=0.14, Mann-Whitney U-test) (FIG. 7E). On average, human ORs were significantly more sensitive than mouse receptors (p<0.008, Mann-Whitney U-test) (FIG. 7F). This finding contradicts the hypothesis that mouse ORs are more sensitive to low-concentration odorants than human ORs.

Using a machine-learning algorithm trained on the physicochemical descriptors of the odorants as above, it was possible to differentiate odorants that activated human Ors from odorants that activated mouse ORs (d′=0.62, p<0.013). The 10 molecular features that best differentiate odorants activating human receptors from odorants activating mouse receptors are shown in Table 1B.

### Predicting Odorant-Receptor Interactions

Except for a few examples (Jacquier et al., J Neurochem 97, 537-44 (2006); Schmiedeberg et al., J Struct Biol 159, 400-12 (2007); Abaffy et al., J Neurochem 97, 1506-18 (2006); Sanz et al., Chemical Senses 33, 639-53 (2008); Araneda et al., Nat Neurosci 3, 1248-55 (2000); Katada et al., J Neurosci 25, 1806-15 (2005); each herein incorporated by reference in their entirety), knowledge of what makes an OR ligand interaction effective is very limited. The present experiments have greatly increased the number of ORs with known agonists. Moreover, all 62 receptors were tested with the same 63 odorants in a consistent assay, allowing for the determination of general rules that govern the interaction between odorant and OR.

Logistic regression was used to differentiate odorant-OR combinations that result in a response from odorant-OR combinations that fail to elicit a response. Both physicochemical descriptors and amino acid properties were combined to predict the interaction of novel odorants with novel ORs.

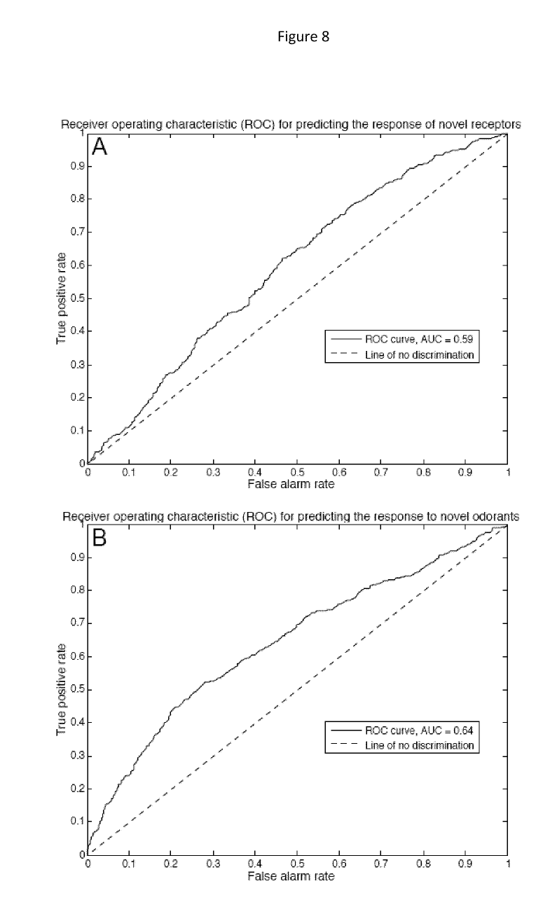

A leave-10-out cross validation procedure was used to validate the model, in each round selecting a set of descriptors that best predicted the training data and applying those descriptors to the test data. The values to leave out were selected using two different methods. First, 10% of the receptors were reserved, simulating a situation in which an investigator is searching for the response of novel ORs to odorants that activate a known OR. The model predicted the response of the ‘novel’ ORs to the 63 odorants [area under the receiver operating characteristic curve (AUC)=0.59, p<0.0001, Mann-Whitney U-test] (FIG. 8A). Second, 10% of the odorants were reserved, simulating a situation in which an investigator is searching for the response of Ors with known agonists to untested odorants. The model predicted the response of all 62 ORs to the ‘novel’ odorants (AUC=0.64, p<0.0001, Mann-Whitney U-test) (FIG. 8B).

TABLE 1
The top 10 physicochemical descriptors for distinguishing between agonists for (A)
class I and class II receptors or (B) human and mouse receptors.

| | Class I | | Class II | |
|---|---|---|---|---|
| A. Top ten descriptors that differentiate | average | | average | |
| class I from class II agonists | value | SE | value | SE |
| Geary autocorrelation - lag 2/weighted by | 0.643 | 0.0021 | 0.733 | 0.0007 |
| atomic masses | | | | |
| Eigenvalue sum from van der Waals weighted | −1.908 | 0.0103 | −0.954 | 0.002 |
| distance matrix | | | | |
| Eigenvalue sum from polarizability weighted | −2.4 | 0.0129 | −1.2 | 0.0026 |
| distance matrix | | | | |
| Radial Distribution Function - 4.5/weighted | 0.748 | 0.0213 | 1.759 | 0.0058 |
| by atomic polarizabilities | | | | |
| 3D-MoRSE - signal 25/unweighted | 0.376 | 0.0036 | 0.534 | 0.0007 |
| 3D-MoRSE - signal 23/weighted by atomic | −0.873 | 0.0067 | −0.445 | 0.0015 |
| Sanderson electronegativities | | | | |
| 2st component symmetry directional WHIM | 0.193 | 0.0008 | 0.23 | 0.0003 |
| index/unweighted | | | | |
| 3rd component accessibility directional WHIM | 0 | 0.0017 | 0.007 | 0.0004 |
| index/weighted by atomic electrotopological | | | | |
| states | | | | |
| H autocorrelation of lag 2/unweighted | 0.989 | 0.0064 | 1.405 | 0.0017 |
| R maximal autocorrelation of lag 2/weighted | 0.024 | 0.0001 | 0.035 | 0 |
| by atomic polarizabilities | | | | |
| | Human | | Mouse | |
| B. Top ten descriptors that differentiate | average | | average | |
| human from mouse agonists | value | SE | value | SE |
| molecular electrotopological variation | 7.347 | 0.0423 | 10.151 | 0.0105 |
| self-returning walk count of order 10 | 2528 | 40.9437 | 5378 | 21.7641 |
| Eigenvalue 01 from edge adjacency atrix | 3.562 | 0.0082 | 3.6245 | 0.0019 |
| weighted by dipole moments | | | | |
| Spectral moment 09 from edge adjacency | 11.4595 | 0.0306 | 11.6935 | 0.0068 |
| matrix weighted by dipole moments | | | | |
| 1st component symmetry directional WHIM | 0.186 | 0.0017 | 0.197 | 0.0007 |
| index/unweighted | | | | |
| 1st component symmetry directional WHIM | 0.177 | 0.0017 | 0.191 | 0.0004 |
| index/weighted by atomic van der Waals | | | | |
| volumes | | | | |
| 1st component symmetry directional WHIM | 0.177 | 0.0017 | 0.191 | 0.0004 |
| index/weighted by atomic Sanderson | | | | |
| electronegativities | | | | |
| leverage-weighted autocorrelation of lag 2/ | 0.055 | 0.001 | 0.076 | 0.0003 |
| weighted by atomic masses | | | | |
| first eigenvalue of the R matrix | 0.8295 | 0.0008 | 0.86 | 0.0003 |
| R autocorrelation of lag 2/weighted by atomic | 0.3085 | 0.0022 | 0.415 | 0.0007 |
| masses | | | | |

TABLE 3

| Odorant Set 1 | Odotant Set 2 | Odorant Set 3 | Odorant set 4 |
|---|---|---|---|
| Pentanoic acid | 4-Hydroxycoumarin | 2-Butanone | tert-Butyl propionate |
| Propionic acid | 4-Chromanone | 2-Pentanone | Methyl butyrate |
| Hexanoic acid | 2-Coumaranone | 2-Hexanone | Propyl butyrate |
| Heptanoic acid | γ-Caprotactone | 2-Heptanone | Pentyl acetate |
| Octanoic Acid | Coumarin | 3-Heptanone | Allyl heptanoate |
| Nonanoic acid | Cyclohexanone | 2-Octanone | Amyl hexanoate |
| Decanoic acid | | 3-Octanone | Amyl butyrate |
| Isovaleric acid | | 2-Nonanone | Butyl heptanoate |
| Thioglycolic acid | | 2,3-Hexanedione | Heptyl isobutyrate |
| Nonanedioic acid | | 3,4-Hexanedione | Hexyl acetate |
| Vanillic acid | | (+)-Carvone | Butyl butyryllactate |
| (+)-2-Phenylbutyric | | (−)-Carvone | Butyl formate |
| acid | | (+)-Dihydrocarvone | Ethyl pyruvate |
| (−)-2-Phenylbutyric | | (−)-Fenchone | Isoamyl acetate |
| acid | | (+)-Fenchone | Ethyl isobutyrate |
| | | (+)-Camphor | Prenyl acetate |
| | | (−)-Camphor | |
| | | Dihydrojasmone | |
| | | Acetophenone | |
| | | Benzophenone | |
| | | (+)-Pulegone | |
| | | 2-Furyl methyl ketone | |
| | | Dimedone | |
| | | (−)-Menthone | |
| | | Ionone | |
| Odorant set 5 | Odorant set 6 | Odorant set 7 | Odorant set 8 |
| 1-Butanol | Allyl phenylacetate | Octanethiol | Butanal |
| 1-Propanol | Benzene | Nonanethiol | Pentanal |
| 1-Pentanol | Benzyl acetate | | Hexanal |
| 1-Hexanol | Allylbenzene | | Heptanal |
| 1-Heptanol | Phenyl acetate | | Octanal |
| 1-Octanol | Prenyl acetate | | Nonanal |
| 1-Nonanol | | | Decanal |
| 1-Decanol | | | Acetal |
| (+)-2-Heptanol | | | Citral |
| (−)-2-Octanol | | | Hydroxycitronellal |
| (+)-2-Octanol | | | Lyral |
| (−)-β-Citronellol | | | |
| Geraniol | | | |
| Linalool | | | |
| 1-Undecanol | | | |

TABLE 4

| | (+)-2-Phenylbutyric acid | (+)-Camphol | (+)-Carvone | (+)-Dihydrocarvone | (+)-Fenchone | (−)-2-Phenylbutric acid | (−)-Camphor |
|---|---|---|---|---|---|---|---|
| MOR1-1 | 0 | 0 | 0 | 0 | 0 | 0 | 0 |
| MOR105-1 | 0 | 0 | 0 | 0 | 0 | 0 | 0 |
| MOR106-1 | 0 | 0 | 0 | 0 | 0 | 0 | 0 |
| MOR107-1 | 0 | −4.15 | 0 | 0 | 0 | 0 | −3.704 |
| MOR128-2 | 0 | −3.772 | 0 | 0 | 0 | 0 | −4.373 |
| MOR129-1 | 0 | 0 | −4.145 | 0 | 0 | 0 | 0 |
| MOR138-1 | 0 | −4.749 | 0 | −3.498 | −4.626 | 0 | −4.952 |
| MOR139-1 | 0 | −3.681 | −4.032 | −3.637 | −3.773 | 0 | −3.902 |
| MOR140-1 | 0 | 0 | 0 | 0 | 0 | 0 | 0 |
| MOR15-1 | 0 | 0 | 0 | 0 | 0 | 0 | 0 |
| MOR161-1 | 0 | 0 | 0 | 0 | 0 | 0 | 0 |
| MOR162-1 | 0 | 0 | 0 | 0 | 0 | 0 | 0 |
| MOR170-1 | 0 | 0 | 0 | 0 | 0 | 0 | 0 |
| MOR18-1 | 0 | 0 | 0 | 0 | 0 | 0 | 0 |
| MOR180-1 | 0 | 0 | 0 | 0 | 0 | 0 | 0 |
| MOR182-1 | 0 | 0 | 0 | −3.406 | 0 | 0 | 0 |
| MOR184-1 | 0 | 0 | −3.812 | −3.55 | 0 | 0 | 0 |
| MOR185-1 | 0 | −3.18 | 0 | 0 | 0 | 0 | 0 |
| MOR189-1 | 0 | −3.631 | −4.302 | −3.976 | −4.423 | 0 | −4.839 |
| MOR2-1 | −3.499 | 0 | 0 | 0 | 0 | −2.982 | 0 |
| MOR203-1 | 0 | 0 | −3.814 | −4.241 | 0 | 0 | 0 |
| MOR204-6 | 0 | 0 | −3.85 | 0 | 0 | 0 | 0 |
| MOR205-1 | 0 | 0 | 0 | 0 | 0 | 0 | 0 |
| MOR207-1 | 0 | 0 | 0 | 0 | 0 | 0 | 0 |
| MOR222-1 | 0 | 0 | 0 | 0 | 0 | 0 | 0 |
| MOR223-1 | 0 | 0 | 0 | 0 | 0 | 0 | 0 |
| MOR23-1 | 0 | 0 | 0 | 0 | 0 | 0 | 0 |
| MOR236-1 | 0 | 0 | 0 | 0 | 0 | 0 | 0 |
| MOR25-1 | 0 | 0 | 0 | 0 | 0 | 0 | 0 |
| MOR250-1 | 0 | 0 | −3.938 | 0 | 0 | 0 | 0 |
| MOR251-1 | 0 | −3.31 | 0 | 0 | 0 | 0 | 0 |
| MOR253-1 | 0 | 0 | 0 | 0 | 0 | 0 | 0 |
| MOR256-17 | 0 | 0 | 0 | 0 | 0 | 0 | 0 |
| MOR258-1 | 0 | 0 | 0 | 0 | 0 | 0 | 0 |
| MOR259-1 | 0 | 0 | 0 | 0 | 0 | 0 | 0 |
| MOR260-1 | 0 | 0 | 0 | 0 | 0 | 0 | 0 |
| MOR261-1 | 0 | 0 | 0 | 0 | 0 | 0 | 0 |
| MOR268-1 | 0 | 0 | 0 | 0 | 0 | 0 | 0 |
| MOR269-1 | 0 | 0 | 0 | 0 | 0 | 0 | 0 |
| MOR271-1 | 0 | 0 | −4.098 | −3.987 | −4.859 | 0 | 0 |
| MOR272-1 | 0 | 0 | −4.564 | −4.273 | −4.35 | 0 | 0 |
| MOR273-1 | 0 | 0 | 0 | 0 | −4.073 | 0 | 0 |
| MOR277-1 | 0 | −3.519 | 0 | 0 | 0 | 0 | −3.861 |
| MOR30-1 | 0 | 0 | 0 | 0 | 0 | 0 | 0 |
| MOR31-1 | 0 | 0 | 0 | 0 | 0 | 0 | 0 |
| MOR33-1 | 0 | 0 | 0 | 0 | 0 | 0 | 0 |
| MOR37-1 | 0 | 0 | 0 | 0 | 0 | 0 | 0 |
| MOR4-1 | 0 | 0 | 0 | 0 | 0 | 0 | 0 |
| MOR40-1 | 0 | 0 | 0 | 0 | 0 | 0 | 0 |
| MOR41-1 | 0 | 0 | −4.644 | −4.301 | 0 | 0 | 0 |
| MOR5-1 | 0 | 0 | 0 | 0 | 0 | 0 | 0 |
| MOR9-1 | 0 | 0 | 0 | 0 | 0 | 0 | 0 |
| OR10J5 | 0 | 0 | 0 | 0 | 0 | 0 | 0 |
| OR1A1 | 0 | 0 | −5.035 | −4.992 | 0 | 0 | 0 |
| OR2C1 | 0 | 0 | 0 | 0 | 0 | 0 | 0 |
| OR2J2 | 0 | 0 | 0 | 0 | 0 | 0 | 0 |
| OR2M7 | 0 | 0 | 0 | 0 | 0 | 0 | 0 |
| OR2W1 | 0 | 0 | −4.646 | −4.755 | 0 | 0 | 0 |
| OR51E1 | 0 | 0 | 0 | 0 | 0 | 0 | 0 |
| OR51E2 | 0 | 0 | 0 | 0 | 0 | 0 | 0 |
| OR51L1 | 0 | 0 | 0 | 0 | 0 | 0 | 0 |
| OR5P3 | 0 | 0 | −5.173 | 0 | 0 | 0 | 0 |

| | (−)-Carvone | (−)-Fenchone | (−)-b-C [symbol] inedol | 1-Decanol | 1-Heptanol | 1-Hexanol | 1-Nononol | 1-Octanol | 1-Pentanol |
|---|---|---|---|---|---|---|---|---|---|
| MOR1-1 | 0 | 0 | 0 | 0 | 0 | 0 | 0 | 0 | 0 |
| MOR105-1 | 0 | 0 | 0 | 0 | 0 | 0 | 0 | 0 | 0 |
| MOR106-1 | 0 | 0 | 0 | 0 | 0 | 0 | 0 | 0 | 0 |
| MOR107-1 | 0 | −4.302 | 0 | 0 | 0 | 0 | 0 | 0 | 0 |
| MOR128-2 | 0 | 0 | 0 | 0 | 0 | 0 | 0 | 0 | 0 |
| MOR129-1 | 0 | 0 | 0 | 0 | 0 | 0 | 0 | 0 | 0 |
| MOR138-1 | 0 | −4.481 | −3.347 | 0 | 0 | 0 | 0 | 0 | 0 |
| MOR139-1 | −3.861 | −3.879 | 0 | 0 | 0 | 0 | 0 | 0 | 0 |
| MOR140-1 | 0 | 0 | 0 | 0 | 0 | 0 | 0 | 0 | 0 |
| MOR15-1 | 0 | 0 | 0 | 0 | 0 | 0 | 0 | 0 | 0 |
| MOR161-1 | 0 | 0 | 0 | 0 | 0 | 0 | 0 | 0 | 0 |
| MOR162-1 | 0 | 0 | 0 | 0 | 0 | 0 | 0 | 0 | 0 |
| MOR170-1 | 0 | 0 | 0 | 0 | 0 | 0 | 0 | 0 | 0 |
| MOR18-1 | 0 | 0 | 0 | 0 | 0 | 0 | 0 | 0 | 0 |
| MOR180-1 | 0 | 0 | 0 | 0 | 0 | 0 | 0 | 0 | 0 |
| MOR182-1 | 0 | 0 | 0 | 0 | 0 | 0 | 0 | 0 | 0 |
| MOR184-1 | −3.92 | 0 | 0 | 0 | 0 | 0 | 0 | 0 | 0 |
| MOR185-1 | 0 | 0 | 0 | 0 | 0 | 0 | 0 | 0 | 0 |
| MOR189-1 | −4.147 | −4.239 | 0 | 0 | 0 | 0 | 0 | 0 | 0 |
| MOR2-1 | 0 | 0 | 0 | 0 | 0 | 0 | 0 | 0 | 0 |
| MOR203-1 | −3.938 | 0 | −3.147 | −3.299 | −3.387 | 0 | −4.456 | −3.563 | 0 |
| MOR204-6 | −3.625 | 0 | 0 | 0 | 0 | 0 | 0 | 0 | 0 |
| MOR205-1 | 0 | 0 | 0 | 0 | 0 | 0 | 0 | 0 | 0 |
| MOR207-1 | 0 | 0 | 0 | 0 | 0 | 0 | 0 | 0 | 0 |
| MOR222-1 | 0 | 0 | 0 | 0 | 0 | 0 | 0 | 0 | 0 |
| MOR223-1 | 0 | 0 | 0 | 0 | 0 | 0 | 0 | 0 | 0 |
| MOR23-1 | 0 | 0 | 0 | 0 | 0 | 0 | 0 | 0 | 0 |
| MOR236-1 | 0 | 0 | 0 | 0 | 0 | 0 | 0 | 0 | 0 |
| MOR25-1 | 0 | 0 | 0 | 0 | 0 | 0 | 0 | 0 | 0 |
| MOR250-1 | 0 | 0 | 0 | 0 | 0 | 0 | 0 | 0 | 0 |
| MOR251-1 | 0 | 0 | 0 | 0 | 0 | 0 | 0 | 0 | 0 |
| MOR253-1 | 0 | 0 | 0 | 0 | 0 | 0 | 0 | 0 | 0 |
| MOR256-17 | 0 | 0 | 0 | 0 | 0 | 0 | 0 | 0 | 0 |
| MOR258-1 | 0 | 0 | 0 | 0 | 0 | 0 | 0 | 0 | 0 |
| MOR259-1 | 0 | 0 | 0 | 0 | 0 | 0 | 0 | 0 | 0 |
| MOR260-1 | 0 | 0 | 0 | 0 | 0 | 0 | 0 | 0 | 0 |
| MOR261-1 | 0 | 0 | 0 | −3.273 | −3.382 | 0 | −4.914 | −5.192 | 0 |
| MOR268-1 | 0 | 0 | 0 | 0 | 0 | 0 | 0 | −3.63 | 0 |
| MOR269-1 | 0 | 0 | 0 | 0 | 0 | 0 | 0 | 0 | 0 |
| MOR271-1 | −4.14 | 0 | 0 | 0 | −3.47 | 0 | 0 | 0 | 0 |
| MOR272-1 | 0 | −3.558 | 0 | 0 | −4.181 | 0 | 0 | 0 | 0 |
| MOR273-1 | 0 | −5.231 | 0 | 0 | 0 | 0 | 0 | 0 | 0 |
| MOR277-1 | 0 | 0 | 0 | 0 | 0 | 0 | 0 | 0 | 0 |
| MOR30-1 | 0 | 0 | 0 | 0 | 0 | 0 | 0 | 0 | 0 |
| MOR31-1 | 0 | 0 | 0 | 0 | 0 | 0 | 0 | 0 | 0 |
| MOR33-1 | 0 | 0 | 0 | 0 | 0 | 0 | 0 | 0 | 0 |
| MOR37-1 | 0 | 0 | 0 | 0 | 0 | 0 | 0 | 0 | 0 |
| MOR4-1 | 0 | 0 | 0 | 0 | 0 | 0 | 0 | 0 | 0 |
| MOR40-1 | 0 | 0 | 0 | 0 | 0 | 0 | 0 | 0 | 0 |
| MOR41-1 | −4.257 | 0 | 0 | 0 | −3.68 | −4.064 | 0 | 0 | −3.537 |
| MOR5-1 | 0 | 0 | 0 | 0 | 0 | 0 | 0 | 0 | 0 |
| MOR9-1 | 0 | 0 | 0 | 0 | 0 | 0 | 0 | 0 | 0 |
| OR10J5 | 0 | 0 | 0 | 0 | 0 | 0 | 0 | 0 | 0 |
| OR1A1 | −5.765 | 0 | −4.039 | −3.082 | −3.013 | 0 | 0 | 0 | 0 |
| OR2C1 | 0 | 0 | 0 | 0 | 0 | 0 | 0 | 0 | 0 |
| OR2J2 | 0 | 0 | 0 | −3.416 | −3.271 | 0 | −4.16 | −4.16 | 0 |
| OR2M7 | 0 | 0 | −3.853 | 0 | 0 | 0 | 0 | 0 | 0 |
| OR2W1 | −4.657 | 0 | −5.108 | −4.365 | −4.831 | −4.852 | −4.63 | −5.128 | 0 |
| OR51E1 | 0 | 0 | 0 | 0 | 0 | 0 | 0 | 0 | 0 |
| OR51E2 | 0 | 0 | 0 | 0 | 0 | 0 | 0 | 0 | 0 |
| OR51L1 | 0 | 0 | 0 | 0 | 0 | 0 | 0 | 0 | 0 |
| OR5P3 | −4.645 | 0 | 0 | 0 | −2.962 | −3.758 | 0 | 0 | 0 |

| | 2-Counpronon [symbol] | 2-Haptonone | 2-Hexanone | 2-Noranone | 2-Octanone | 2-Penonone | 23-Haxanedione | 3-Haptonone |
|---|---|---|---|---|---|---|---|---|
| MOR1-1 | −4.138 | 0 | 0 | 0 | 0 | 0 | 0 | 0 |
| MOR105-1 | 0 | 0 | 0 | 0 | 0 | 0 | 0 | 0 |
| MOR106-1 | 0 | 0 | 0 | 0 | 0 | 0 | 0 | 0 |
| MOR107-1 | 0 | 0 | 0 | 0 | 0 | 0 | 0 | 0 |
| MOR126-2 | 0 | 0 | 0 | 0 | 0 | 0 | 0 | 0 |
| MOR129-1 | 0 | 0 | 0 | 0 | 0 | 0 | 0 | 0 |
| MOR136-1 | 0 | 0 | 0 | 0 | 0 | 0 | 0 | 0 |
| MOR139-1 | −3.585 | 0 | 0 | 0 | 0 | 0 | 0 | 0 |
| MOR140-1 | 0 | 0 | 0 | 0 | 0 | 0 | 0 | 0 |
| MOR15-1 | 0 | 0 | 0 | 0 | 0 | 0 | 0 | 0 |
| MOR161-1 | 0 | 0 | 0 | 0 | 0 | 0 | 0 | 0 |
| MOR162-1 | 0 | 0 | 0 | 0 | 0 | 0 | 0 | 0 |
| MOR170-1 | 0 | 0 | 0 | 0 | 0 | 0 | 0 | 0 |
| MOR18-1 | 0 | 0 | 0 | 0 | 0 | 0 | 0 | 0 |
| MOR180-1 | 0 | 0 | 0 | 0 | 0 | 0 | 0 | 0 |
| MOR182-1 | 0 | 0 | 0 | 0 | 0 | 0 | 0 | 0 |
| MOR184-1 | 0 | 0 | 0 | 0 | 0 | 0 | 0 | 0 |
| MOR185-1 | 0 | 0 | 0 | 0 | 0 | 0 | 0 | 0 |
| MOR189-1 | 0 | 0 | 0 | 0 | 0 | 0 | 0 | 0 |
| MOR2-1 | 0 | 0 | 0 | 0 | 0 | 0 | 0 | 0 |
| MOR203-1 | 0 | −4.76 | 0 | −5.064 | −5.023 | 0 | 0 | −4.099 |
| MOR204-6 | 0 | 0 | 0 | 0 | 0 | 0 | 0 | 0 |
| MOR205-1 | 0 | 0 | 0 | 0 | 0 | 0 | 0 | 0 |
| MOR207-1 | 0 | 0 | 0 | 0 | 0 | 0 | 0 | 0 |
| MOR222-1 | 0 | 0 | 0 | 0 | 0 | 0 | 0 | 0 |
| MOR223-1 | 0 | 0 | 0 | 0 | 0 | 0 | 0 | 0 |
| MOR23-1 | 0 | 0 | 0 | 0 | 0 | 0 | 0 | 0 |
| MOR236-1 | 0 | 0 | 0 | 0 | 0 | 0 | 0 | 0 |
| MOR25-1 | 0 | 0 | 0 | 0 | 0 | 0 | 0 | 0 |
| MOR250-1 | 0 | 0 | 0 | 0 | 0 | 0 | 0 | 0 |
| MOR251-1 | 0 | 0 | 0 | 0 | 0 | 0 | 0 | 0 |
| MOR253-1 | 0 | 0 | 0 | 0 | 0 | 0 | 0 | 0 |
| MOR256-17 | −4.575 | 0 | 0 | 0 | 0 | 0 | −4.838 | 0 |
| MOR258-1 | −4.268 | 0 | 0 | 0 | 0 | 0 | 0 | 0 |
| MOR259-1 | −3.63 | 0 | 0 | 0 | 0 | 0 | 0 | 0 |
| MOR260-1 | 0 | 0 | 0 | 0 | 0 | 0 | 0 | 0 |
| MOR261-1 | 0 | 0 | 0 | 0 | 0 | 0 | 0 | 0 |
| MOR268-1 | 0 | 0 | 0 | 0 | 0 | 0 | 0 | 0 |
| MOR269-1 | 0 | 0 | 0 | 0 | 0 | 0 | 0 | 0 |
| MOR271-1 | 0 | −4.581 | −4.936 | 0 | 0 | −5.111 | −4.824 | −5.235 |
| MOR272-1 | 0 | −5.073 | −4.251 | 0 | −4.575 | 0 | 0 | −5.221 |
| MOR273-1 | 0 | 0 | 0 | 0 | 0 | 0 | 0 | 0 |
| MOR277-1 | 0 | 0 | 0 | 0 | 0 | 0 | 0 | 0 |
| MOR30-1 | 0 | 0 | 0 | 0 | 0 | 0 | 0 | 0 |
| MOR31-1 | 0 | 0 | 0 | 0 | 0 | 0 | 0 | 0 |
| MOR33-1 | 0 | 0 | 0 | 0 | 0 | 0 | 0 | 0 |
| MOR37-1 | 0 | 0 | 0 | 0 | 0 | 0 | 0 | 0 |
| MOR4-1 | 0 | 0 | 0 | 0 | 0 | 0 | 0 | 0 |
| MOR40-1 | 0 | 0 | 0 | 0 | 0 | 0 | 0 | 0 |
| MOR41-1 | −3.96 | 0 | 0 | 0 | 0 | 0 | −5.008 | 0 |
| MOR5-1 | 0 | 0 | 0 | 0 | 0 | 0 | 0 | 0 |
| MOR9-1 | 0 | 0 | 0 | 0 | 0 | 0 | 0 | 0 |
| OR10J5 | 0 | 0 | 0 | 0 | 0 | 0 | 0 | 0 |
| OR1A1 | 0 | −4.399 | 0 | −3.577 | −4.37 | 0 | 0 | −4.712 |
| OR2C1 | 0 | 0 | 0 | 0 | 0 | 0 | 0 | 0 |
| OR2J2 | 0 | 0 | 0 | 0 | 0 | 0 | 0 | 0 |
| OR2M7 | 0 | 0 | 0 | 0 | 0 | 0 | 0 | 0 |
| OR2W1 | 0 | −5.142 | −4.135 | −4.701 | −4.399 | 0 | −3.363 | −4.477 |
| OR51E1 | 0 | 0 | 0 | 0 | 0 | 0 | 0 | 0 |
| OR51E2 | 0 | 0 | 0 | 0 | 0 | 0 | 0 | 0 |
| OR51L1 | 0 | 0 | 0 | 0 | 0 | 0 | 0 | 0 |
| OR5P3 | 0 | 0 | 0 | 0 | 0 | 0 | 0 | 0 |

| | 3-Octanone | 34-Hexanedione | 4-Chromanone | 4-Hydroxycoumarin | Acetophenone | Allyl benzene | Allyl heptanoate |
|---|---|---|---|---|---|---|---|
| MOR1-1 | 0 | 0 | 0 | 0 | 0 | 0 | 0 |
| MOR105-1 | 0 | 0 | 0 | 0 | −3.429 | −3.332 | 0 |
| MOR106-1 | 0 | 0 | 0 | 0 | −3.854 | −3.921 | 0 |
| MOR107-1 | 0 | 0 | 0 | 0 | 0 | 0 | 0 |
| MOR128-2 | 0 | 0 | 0 | 0 | 0 | 0 | 0 |
| MOR129-1 | 0 | 0 | −3.785 | 0 | −5.484 | 0 | 0 |
| MOR138-1 | 0 | 0 | 0 | 0 | 0 | 0 | 0 |
| MOR139-1 | 0 | 0 | −4.028 | 0 | −3.935 | 0 | 0 |
| MOR140-1 | 0 | 0 | 0 | 0 | 0 | 0 | 0 |
| MOR15-1 | 0 | 0 | 0 | 0 | 0 | 0 | 0 |
| MOR161-1 | 0 | 0 | 0 | 0 | −3.825 | 0 | 0 |
| MOR162-1 | 0 | 0 | −4.225 | 0 | −4.778 | 0 | 0 |
| MOR170-1 | 0 | 0 | −4.209 | 0 | −3.9 | 0 | 0 |
| MOR18-1 | 0 | 0 | 0 | 0 | 0 | 0 | 0 |
| MOR180-1 | 0 | 0 | 0 | 0 | 0 | 0 | 0 |
| MOR182-1 | 0 | 0 | 0 | 0 | 0 | 0 | 0 |
| MOR184-1 | 0 | 0 | 0 | 0 | 0 | 0 | 0 |
| MOR185-1 | 0 | 0 | −3.833 | 0 | −4.016 | 0 | 0 |
| MOR189-1 | 0 | 0 | −3.684 | 0 | −3.372 | 0 | 0 |
| MOR2-1 | 0 | 0 | 0 | 0 | 0 | 0 | 0 |
| MOR203-1 | −4.4 | 0 | 0 | 0 | 0 | 0 | 0 |
| MOR204-6 | 0 | 0 | 0 | 0 | 0 | 0 | 0 |
| MOR205-1 | 0 | 0 | 0 | 0 | 0 | 0 | 0 |
| MOR207-1 | 0 | 0 | −3.724 | 0 | −3.608 | 0 | 0 |
| MOR222-1 | 0 | 0 | −3.618 | 0 | 0 | 0 | 0 |
| MOR223-1 | 0 | 0 | 0 | 0 | 0 | 0 | 0 |
| MOR23-1 | 0 | 0 | 0 | 0 | 0 | 0 | 0 |
| MOR236-1 | 0 | 0 | 0 | 0 | 0 | 0 | 0 |
| MOR25-1 | 0 | 0 | 0 | 0 | 0 | 0 | 0 |
| MOR250-1 | 0 | 0 | 0 | 0 | −3.975 | 0 | 0 |
| MOR251-1 | 0 | 0 | 0 | 0 | 0 | 0 | 0 |
| MOR253-1 | o | 0 | 0 | 0 | 0 | 0 | 0 |
| MOR256-17 | 0 | −4.921 | 0 | 0 | 0 | −4.146 | 0 |
| MOR258-1 | 0 | 0 | 0 | 0 | −3.358 | 0 | 0 |
| MOR259-1 | 0 | 0 | 0 | 0 | 0 | 0 | 0 |
| MOR260-1 | 0 | 0 | 0 | 0 | 0 | 0 | 0 |
| MOR261-1 | 0 | 0 | 0 | 0 | 0 | 0 | 0 |
| MOR268-1 | 0 | 0 | 0 | 0 | 0 | 0 | 0 |
| MOR269-1 | 0 | 0 | 0 | 0 | 0 | 0 | 0 |
| MOR271-1 | −4.139 | −4.734 | −4.047 | 0 | −4.05 | 0 | 0 |
| MOR272-1 | −4.197 | 0 | −4.043 | 0 | −3.922 | 0 | 0 |
| MOR273-1 | 0 | 0 | −4.201 | 0 | 0 | 0 | 0 |
| MOR277-1 | 0 | 0 | −3.415 | 0 | 0 | 0 | 0 |
| MOR30-1 | 0 | 0 | 0 | 0 | 0 | 0 | 0 |
| MOR31-1 | 0 | 0 | 0 | 0 | 0 | 0 | 0 |
| MOR33-1 | 0 | 0 | 0 | 0 | 0 | 0 | 0 |
| MOR37-1 | 0 | 0 | 0 | 0 | 0 | 0 | 0 |
| MOR4-1 | 0 | 0 | 0 | 0 | 0 | 0 | 0 |
| MOR40-1 | 0 | 0 | 0 | 0 | 0 | 0 | 0 |
| MOR41-1 | 0 | −4.692 | −4.747 | −4.344 | −5.039 | 0 | 0 |
| MOR5-1 | 0 | 0 | 0 | 0 | 0 | 0 | 0 |
| MOR9-1 | 0 | 0 | 0 | 0 | 0 | 0 | 0 |
| OR10J5 | 0 | 0 | 0 | 0 | 0 | 0 | 0 |
| OR1A1 | −5.291 | 0 | −3.335 | 0 | 0 | 0 | −3.323 |
| OR2C1 | 0 | 0 | 0 | 0 | 0 | 0 | 0 |
| OR2J2 | 0 | 0 | 0 | 0 | 0 | 0 | 0 |
| OR2M7 | 0 | 0 | 0 | 0 | 0 | 0 | 0 |
| OR2W1 | −5.02 | −4.78 | −4.041 | 0 | −4.372 | 0 | 0 |
| OR51E1 | 0 | 0 | 0 | 0 | 0 | 0 | 0 |
| OR51E2 | 0 | 0 | 0 | 0 | 0 | 0 | 0 |
| OR51L1 | 0 | 0 | 0 | 0 | 0 | 0 | 0 |
| OR5P3 | 0 | 0 | 0 | 0 | −3.898 | 0 | 0 |

| | Allyl phenylacetate | Amyl hexanote | Benzene | Benzophenone | Benzyl acetate | Butyl butyryl actate | Butyl formate | Coumarin |
|---|---|---|---|---|---|---|---|---|
| MOR1-1 | −3.581 | 0 | 0 | 0 | 0 | 0 | 0 | 0 |
| MOR105-1 | 0 | 0 | 0 | 0 | 0 | 0 | 0 | 0 |
| MOR106-1 | 0 | 0 | 0 | 0 | 0 | 0. | 0 | −3.919 |
| MOR107-1 | 0 | 0 | 0 | 0 | 0 | 0 | 0 | 0 |
| MOR128-2 | 0 | 0 | 0 | 0 | 0 | 0 | 0 | 0 |
| MOR129-1 | 0 | 0 | 0 | 0 | 0 | 0 | 0 | −4.04 |
| MOR138-1 | 0 | 0 | 0 | 0 | 0 | 0 | 0 | 0 |
| MOR139-1 | −3.625 | 0 | 0 | 0 | −3.729 | 0 | 0 | −3.749 |
| MOR140-1 | 0 | 0 | 0 | 0 | 0 | 0 | 0 | 0 |
| MOR15-1 | 0 | 0 | 0 | 0 | 0 | 0 | 0 | 0 |
| MOR161-1 | 0 | 0 | 0 | 0 | 0 | 0 | 0 | −3.139 |
| MOR162-1 | 0 | 0 | 0 | −3.685 | 0 | 0 | 0 | −4.483 |
| MOR170-1 | 0 | 0 | 0 | 0 | 0 | 0 | 0 | −3.989 |
| MOR18-1 | 0 | 0 | 0 | 0 | 0 | 0 | 0 | 0 |
| MOR180-1 | 0 | 0 | 0 | 0 | 0 | 0 | 0 | 0 |
| MOR182-1 | 0 | 0 | 0 | 0 | 0 | 0 | 0 | 0 |
| MOR184-1 | 0 | 0 | 0 | 0 | 0 | 0 | 0 | 0 |
| MOR185-1 | 0 | 0 | 0 | 0 | 0 | 0 | 0 | −4.042 |
| MOR189-1 | 0 | 0 | 0 | 0 | 0 | 0 | 0 | −3.215 |
| MOR2-1 | −3.253 | 0 | 0 | 0 | 0 | 0 | 0 | 0 |
| MOR203-1 | −3.401 | 0 | 0 | 0 | −3.531 | 0 | 0 | 0 |
| MOR204-6 | 0 | 0 | 0 | 0 | 0 | 0 | 0 | −5.099 |
| MOR205-1 | 0 | 0 | 0 | 0 | 0 | 0 | 0 | 0 |
| MOR207-1 | 0 | 0 | 0 | 0 | 0 | 0 | 0 | −3.905 |
| MOR222-1 | −3.369 | 0 | 0 | 0 | 0 | 0 | 0 | 0 |
| MOR223-1 | −4.178 | 0 | 0 | 0 | −3.841 | 0 | 0 | 0 |
| MOR23-1 | 0 | 0 | 0 | 0 | 0 | 0 | 0 | 0 |
| MOR236-1 | −4.052 | 0 | 0 | 0 | 0 | 0 | 0 | 0 |
| MOR25-1 | 0 | 0 | 0 | 0 | 0 | 0 | 0 | 0 |
| MOR250-1 | 0 | 0 | 0 | 0 | 0 | 0 | 0 | −3.855 |
| MOR251-1 | 0 | 0 | 0 | 0 | 0 | 0 | 0 | 0 |
| MOR253-1 | 0 | 0 | 0 | 0 | 0 | 0 | 0 | 0 |
| MOR258-17 | −3.842 | −4.462 | 0 | 0 | 0 | 0 | 0 | 0 |
| MOR258-1 | 0 | 0 | 0 | 0 | 0 | 0 | 0 | −4.296 |
| MOR259-1 | 0 | 0 | 0 | −4.442 | 0 | 0 | 0 | −3.834 |
| MOR260-1 | 0 | 0 | 0 | 0 | 0 | 0 | 0 | 0 |
| MOR261-1 | −3.425 | 0 | 0 | 0 | −3.352 | 0 | 0 | 0 |
| MOR268-1 | −3.434 | 0 | 0 | 0 | 0 | 0 | 0 | 0 |
| MOR269-1 | 0 | 0 | 0 | −3.652 | 0 | 0 | 0 | 0 |
| MOR271-1 | −4.23 | 0 | −4.005 | 0 | −5.379 | 0 | 0 | 0 |
| MOR272-1 | −4.824 | 0 | 0 | 0 | −4.948 | 0 | 0 | 0 |
| MOR273-1 | 0 | 0 | 0 | 0 | 0 | 0 | 0 | 0 |
| MOR277-1 | 0 | 0 | 0 | 0 | 0 | 0 | 0 | 0 |
| MOR30-1 | 0 | 0 | 0 | 0 | 0 | 0 | 0 | 0 |
| MOR31-1 | 0 | 0 | 0 | 0 | 0 | 0 | 0 | 0 |
| MOR33-1 | 0 | 0 | 0 | 0 | 0 | 0 | 0 | 0 |
| MOR37-1 | 0 | 0 | 0 | 0 | 0 | 0 | 0 | 0 |
| MOR4-1 | 0 | 0 | 0 | 0 | 0 | 0 | 0 | 0 |
| MOR40-1 | 0 | 0 | 0 | 0 | 0 | 0 | 0 | 0 |
| MOR41-1 | 0 | 0 | 0 | 0 | −3.972 | 0 | 0 | −4.785 |
| MOR5-1 | 0 | 0 | 0 | 0 | 0 | 0 | 0 | 0 |
| MOR9-1 | 0 | 0 | 0 | 0 | 0 | 0 | 0 | 0 |
| OR10J5 | 0 | 0 | 0 | 0 | 0 | 0 | 0 | 0 |
| OR1A1 | −5.119 | 0 | 0 | −4.008 | −3.391 | 0 | 0 | 0 |
| OR2C1 | 0 | 0 | 0 | 0 | 0 | 0 | 0 | 0 |
| OR2J2 | 0 | 0 | 0 | 0 | 0 | 0 | 0 | −3.562 |
| OR2M7 | 0 | 0 | 0 | 0 | 0 | 0 | 0 | 0 |
| OR2W1 | −4.812 | 0 | 0 | −4.54 | −4.786 | 0 | −4.427 | 4.065 |
| OR51E1 | 0 | 0 | 0 | 0 | 0 | −3.473 | 0 | 0 |
| OR51E2 | 0 | 0 | 0 | 0 | 0 | 0 | 0 | 0 |
| OR51L1 | −4.17 | 0 | 0 | 0 | 0 | 0 | 0 | 0 |
| OR5P3 | 0 | 0 | 0 | 0 | 0 | 0 | 0 | −5.25 |

| | Cyclohexanone | Daconal | Decanoic acid | Dihydroiasmone | Ethyl isobutyrate | Getaniol | Heptonal | Heptanoic acid | Hexanol |
|---|---|---|---|---|---|---|---|---|---|
| MOR1-1 | 0 | 0 | 0 | 0 | 0 | 0 | 0 | −3.823 | −3.987 |
| MOR105-1 | 0 | 0 | 0 | 0 | 0 | 0 | 0 | 0 | 0 |
| MOR106-1 | 0 | 0 | 0 | 0 | 0 | 0 | 0 | 0 | 0 |
| MOR107-1 | 0 | 0 | 0 | 0 | 0 | 0 | 0 | 0 | 0 |
| MOR128-2 | 0 | 0 | 0 | 0 | 0 | 0 | 0 | 0 | 0 |
| MOR129-1 | 0 | 0 | 0 | 0 | 0 | 0 | 0 | 0 | 0 |
| MOR136-1 | −3.065 | 0 | 0 | 0 | 0 | 0 | 0 | 0 | 0 |
| MOR139-1 | −3.138 | 0 | 0 | −4.308 | 0 | −3.939 | 0 | 0 | 0 |
| MOR140-1 | 0 | 0 | 0 | −3.614 | 0 | 0 | 0 | 0 | 0 |
| MOR15-1 | 0 | 0 | 0 | 0 | 0 | 0 | 0 | 0 | 0 |
| MOR161-1 | 0 | 0 | 0 | 0 | 0 | 0 | 0 | 0 | 0 |
| MOR162-1 | 0 | 0 | 0 | 0 | 0 | 0 | 0 | 0 | 0 |
| MOR170-1 | 0 | 0 | 0 | 0 | 0 | 0 | 0 | 0 | 0 |
| MOR18-1 | 0 | 0 | 0 | 0 | 0 | 0 | 0 | 0 | 0 |
| MOR180-1 | 0 | 0 | 0 | 0 | 0 | 0 | 0 | 0 | 0 |
| MOR182-1 | 0 | 0 | 0 | 0 | 0 | 0 | 0 | 0 | 0 |
| MOR184-1 | 0 | 0 | 0 | 0 | 0 | 0 | 0 | 0 | 0 |
| MOR185-1 | −3.594 | 0 | 0 | 0 | 0 | 0 | 0 | 0 | 0 |
| MOR189-1 | 0 | 0 | 0 | −3.945 | 0 | 0 | 0 | 0 | 0 |
| MOR2-1 | 0 | 0 | 0 | 0 | 0 | 0 | 0 | 0 | 0 |
| MOR203-1 | 0 | 0 | −3.751 | 0 | 0 | −3.622 | 0 | 0 | 0 |
| MOR204-6 | 0 | 0 | 0 | 0 | 0 | 0 | 0 | 0 | 0 |
| MOR205-1 | 0 | 0 | 0 | −4.699 | 0 | 0 | 0 | 0 | 0 |
| MOR207-1 | 0 | 0 | 0 | 0 | 0 | 0 | 0 | 0 | 0 |
| MOR222-1 | 0 | 0 | 0 | 0 | 0 | 0 | 0 | 0 | 0 |
| MOR223-1 | 0 | 0 | 0 | 0 | 0 | 0 | 0 | 0 | 0 |
| MOR23-1 | 0 | 0 | 0 | 0 | 0 | 0 | 0 | −4.856 | 0 |
| MOR238-1 | 0 | 0 | 0 | 0 | 0 | −3.843 | 0 | 0 | 0 |
| MOR25-1 | 0 | 0 | −4.194 | 0 | 0 | 0 | 0 | 0 | 0 |
| MOR250-1 | 0 | 0 | 0 | 0 | 0 | 0 | 0 | 0 | 0 |
| MOR251-1 | −3.193 | 0 | 0 | 0 | 0 | 0 | 0 | 0 | 0 |
| MOR253-1 | −3.219 | 0 | 0 | 0 | 0 | 0 | 0 | 0 | 0 |
| MOR258-17 | −4.1 | 0 | −4.627 | 0 | 0 | 0 | 0 | 0 | 0 |
| MOR258-1 | −3.824 | 0 | 0 | 0 | 0 | 0 | 0 | 0 | 0 |
| MOR259-1 | 0 | 0 | 0 | 0 | 0 | 0 | 0 | 0 | 0 |
| MOR260-1 | 0 | 0 | −3.542 | 0 | 0 | 0 | 0 | 0 | 0 |
| MOR261-1 | 0 | 0 | 0 | 0 | 0 | 0 | 0 | 0 | 0 |
| MOR268-1 | 0 | 0 | 0 | 0 | 0 | −3.897 | 0 | 0 | 0 |
| MOR269-1 | 0 | 0 | 0 | 0 | 0 | 0 | 0 | 0 | 0 |
| MOR271-1 | −3.463 | 0 | 0 | −4.126 | −4.077 | 0 | 0 | 0 | −3.322 |
| M0R272-1 | −3.254 | 0 | 0 | −4.447 | −5.25 | 0 | 0 | 0 | 0 |
| MOR273-1 | −3.208 | 0 | 0 | −4.441 | −5.761 | 0 | 0 | 0 | 0 |
| MOR277-1 | −3.568 | 0 | 0 | 0 | 0 | 0 | 0 | 0 | 0 |
| MOR30-1 | 0 | −4.033 | −4.987 | 0 | 0 | 0 | 0 | −3.575 | 0 |
| MOR31-1 | 0 | 0 | 0 | 0 | 0 | 0 | 0 | −5.2 | 0 |
| MOR33-1 | 0 | 0 | −4.487 | 0 | 0 | 0 | 0 | −4.148 | 0 |
| MOR37-1 | 0 | −3.477 | −4.323 | 0 | 0 | 0 | 0 | 0 | 0 |
| MOR4-1 | 0 | 0 | 0 | 0 | 0 | 0 | 0 | 0 | −3.231 |
| MOR40-1 | 0 | 0 | −4.121 | 0 | 0 | 0 | 0 | 0 | 0 |
| MOR41-1 | −3.456 | 0 | 0 | −3.766 | 0 | 0 | 0 | 0 | 0 |
| MOR5-1 | 0 | 0 | −3.749 | 0 | 0 | 0 | 0 | −3.583 | 0 |
| MOR9-1 | 0 | 0 | 0 | 0 | 0 | 0 | 0 | 0 | 0 |
| OR10J5 | 0 | 0 | 0 | 0 | 0 | 0 | 0 | 0 | 0 |
| OR1A1 | 0 | 0 | 0 | −7.242 | 0 | −3.467 | 0 | 0 | 0 |
| OR2C1 | 0 | 0 | 0 | 0 | 0 | 0 | 0 | 0 | 0 |
| OR2J2 | 0 | 0 | 0 | 0 | 0 | 0 | 0 | 0 | 0 |
| OR2M7 | 0 | 0 | 0 | 0 | 0 | −3.203 | 0 | 0 | 0 |
| OR2W1 | 0 | 0 | −3.758 | −4.246 | 0 | −4.736 | −4.056 | 0 | −5.102 |
| OR51E1 | 0 | 0 | 0 | 0 | 0 | 0 | 0 | 0 | 0 |
| OR51E2 | 0 | 0 | 0 | 0 | 0 | 0 | 0 | 0 | 0 |
| OR51L1 | 0 | 0 | 0 | 0 | 0 | 0 | 0 | 0 | 0 |
| OR5P3 | 0 | 0 | 0 | 0 | 0 | 0 | 0 | 0 | 0 |

| | Hexanoic acid | Hexyl acetate | Lyral | Nonanal | Nonanethiol | Nonanoic acid | Octanal | Octanethiol | Octanoic acid |
|---|---|---|---|---|---|---|---|---|---|
| MOR1-1 | −4.625 | 0 | 0 | 0 | 0 | 0 | 0 | 0 | 0 |
| MOR105-1 | 0 | 0 | 0 | 0 | 0 | 0 | 0 | 0 | 0 |
| MOR106-1 | 0 | 0 | 0 | 0 | 0 | 0 | 0 | 0 | 0 |
| MOR107-1 | 0 | 0 | 0 | 0 | 0 | 0 | 0 | 0 | 0 |
| MOR128-2 | 0 | 0 | 0 | 0 | 0 | 0 | 0 | 0 | 0 |
| MOR129-1 | 0 | 0 | 0 | 0 | 0 | 0 | 0 | 0 | 0 |
| MOR138-1 | 0 | 0 | 0 | 0 | 0 | 0 | 0 | 0 | 0 |
| MOR139-1 | 0 | 0 | 0 | 0 | 0 | 0 | 0 | 0 | 0 |
| MOR140-1 | 0 | 0 | 0 | 0 | 0 | 0 | 0 | 0 | 0 |
| MOR15-1 | 0 | 0 | 0 | 0 | 0 | 0 | 0 | 0 | 0 |
| MOR161-1 | 0 | 0 | 0 | 0 | 0 | 0 | 0 | 0 | 0 |
| MOR162-1 | 0 | 0 | 0 | 0 | 0 | 0 | 0 | 0 | 0 |
| MOR170-1 | 0 | 0 | 0 | 0 | 0 | 0 | 0 | 0 | 0 |
| MOR18-1 | 0 | 0 | 0 | 0 | 0 | −4.077 | 0 | 0 | 0 |
| MOR180-1 | 0 | 0 | 0 | 0 | 0 | 0 | 0 | 0 | 0 |
| MOR182-1 | 0 | 0 | 0 | 0 | 0 | 0 | 0 | 0 | 0 |
| MOR184-1 | 0 | 0 | 0 | 0 | 0 | 0 | 0 | 0 | 0 |
| MOR185-1 | 0 | 0 | 0 | 0 | 0 | 0 | 0 | 0 | 0 |
| MOR189-1 | 0 | 0 | 0 | 0 | 0 | 0 | 0 | 0 | 0 |
| MOR2-1 | 0 | 0 | 0 | 0 | 0 | 0 | 0 | 0 | 0 |
| MOR203-1 | 0 | 0 | 0 | 0 | −3.528 | −3.478 | 0 | −3.356 | 0 |
| MOR204-6 | 0 | 0 | 0 | 0 | 0 | 0 | 0 | 0 | 0 |
| MOR205-1 | 0 | 0 | 0 | 0 | 0 | 0 | 0 | 0 | 0 |
| MOR207-1 | 0 | 0 | 0 | 0 | 0 | 0 | 0 | 0 | 0 |
| MOP222-1 | 0 | 0 | 0 | 0 | 0 | 0 | 0 | 0 | 0 |
| MOR223-1 | 0 | 0 | 0 | 0 | 0 | 0 | 0 | 0 | 0 |
| MOR23-1 | 0 | 0 | 0 | 0 | 9 | 0 | 0 | 0 | −5.053 |
| MOR236-1 | 0 | 0 | 0 | 0 | 0 | 0 | 0 | 0 | 0 |
| MOR25-1 | 0 | 0 | 0 | 0 | 0 | 0 | 0 | 0 | −3.689 |
| MOR250-1 | 0 | 0 | 0 | 0 | 0 | 0 | 0 | 0 | 0 |
| MOR251-1 | 0 | 0 | 0 | 0 | 0 | 0 | 0 | 0 | 0 |
| MOR253-1 | 0 | 0 | 0 | 0 | 0 | 0 | 0 | 0 | 0 |
| MOR256-17 | 0 | 0 | −4.255 | 0 | 0 | −4.694 | 0 | −4.388 | −4.41 |
| MOR258-1 | 0 | 0 | 0 | 0 | 0 | 0 | 0 | 0 | 0 |
| MOR259-1 | 0 | 0 | 0 | 0 | 0 | 0 | 0 | 0 | 0 |
| MOR260-1 | 0 | 0 | 0 | 0 | −4.488 | −4.108 | 0 | 0 | 0 |
| MOR261-1 | 0 | 0 | 0 | 0 | −3.376 | 0 | 0 | 0 | 0 |
| MOR268-1 | 0 | 0 | 0 | 0 | −3.589 | 0 | 0 | 0 | 0 |
| MOR269-1 | 0 | 0 | 0 | 0 | 0 | 0 | 0 | 0 | 0 |
| MOR271-1 | 0 | 0 | 0 | 0 | 0 | 0 | 0 | 0 | 0 |
| MOR272-1 | 0 | 0 | 0 | 0 | 0 | 0 | 0 | 0 | 0 |
| MOR273-1 | 0 | 0 | 0 | 0 | 0 | 0 | 0 | 0 | 0 |
| MOR277-1 | 0 | 0 | 0 | 0 | 0 | 0 | 0 | 0 | 0 |
| MOR30-1 | 0 | 0 | 0 | −3.914 | 0 | −4.396 | 0 | 0 | −3.68 |
| MOR31-1 | 0 | 0 | 0 | 0 | 0 | 0 | 0 | 0 | 0 |
| MOR33-1 | 0 | 0 | 0 | −3.234 | 0 | −4.606 | 0 | 0 | −4.212 |
| MOR37-1 | 0 | 0 | 0 | −3.295 | 0 | −3.9 | 0 | 0 | 0 |
| MOR4-1 | 0 | 0 | 0 | 0 | 0 | 0 | 0 | 0 | 0 |
| MOR40-1 | 0 | 0 | 0 | −3.194 | 0 | −4.128 | 0 | 0 | −3.617 |
| MOR41-1 | 0 | 0 | 0 | 0 | 0 | 0 | 0 | 0 | 0 |
| MOR5-1 | 0 | 0 | 0 | 0 | 0 | −4.029 | 0 | 0 | −3.711 |
| MOR9-1 | 0 | 0 | 0 | 0 | 0 | 0 | 0 | 0 | 0 |
| OR10J5 | 0 | 0 | −3.489 | 0 | 0 | 0 | 0 | 0 | 0 |
| OR1A1 | 0 | 0 | 0 | 0 | −3.243 | 0 | 0 | −3.226 | 0 |
| OR2C1 | 0 | 0 | 0 | 0 | −3.893 | 0 | 0 | −4.051 | 0 |
| OR2J2 | 0 | 0 | 0 | 0 | 0 | 0 | 0 | 0 | 0 |
| OR2M7 | 0 | 0 | 0 | 0 | 0 | 0 | 0 | 0 | 0 |
| OR2W1 | 0 | −4.863 | 0 | −3.598 | −4.065 | −3.723 | −4.361 | −4.033 | −4.847 |
| OR51E1 | 0 | 0 | 0 | 0 | 0 | −3.609 | 0 | 0 | 0 |
| OR51E2 | 0 | 0 | 0 | 0 | 0 | 0 | 0 | 0 | 0 |
| OR51L1 | −3.834 | 0 | 0 | 0 | 0 | 0 | 0 | 0 | 0 |
| OR5P3 | 0 | 0 | 0 | 0 | 0 | 0 | 0 | 0 | 0 |

| | Pentanoic acid | Phenyl acetate | Phenyl acetate | Propionic acid | Vanillic acid | α-Caprotactone |
|---|---|---|---|---|---|---|
| MOR1-1 | −4.813 | 0 | 0 | 0 | 0 | 0 |
| MOR105-1 | 0 | 0 | 0 | 0 | 0 | 0 |
| MOR106-1 | 0 | −3.874 | 0 | 0 | 0 | 0 |
| MOR107-1 | 0 | 0 | 0 | 0 | 0 | 0 |
| MOR128-2 | 0 | 0 | 0 | 0 | 0 | 0 |
| MOR129-1 | 0 | 0 | 0 | 0 | 0 | 0 |
| MOR138-1 | 0 | 0 | 0 | 0 | 0 | 0 |
| MOR139-1 | 0 | −3.421 | −3.385 | 0 | 0 | 0 |
| MOR140-1 | 0 | 0 | 0 | 0 | 0 | 0 |
| MOR15-1 | 0 | 0 | 0 | 0 | −3.587 | 0 |
| MOR161-1 | 0 | 0 | 0 | 0 | 0 | 0 |
| MOR162-1 | 0 | 0 | 0 | 0 | 0 | 0 |
| MOR170-1 | 0 | 0 | 0 | 0 | 0 | 0 |
| MOR18-1 | 0 | 0 | 0 | 0 | 0 | 0 |
| MOR180-1 | 0 | 0 | −3.18 | 0 | 0 | 0 |
| MOR182-1 | 0 | 0 | 0 | 0 | 0 | 0 |
| MOR184-1 | 0 | 0 | 0 | 0 | 0 | 0 |
| MOR185-1 | 0 | 0 | 0 | 0 | 0 | −3.185 |
| MOR189-1 | 0 | 0 | 0 | 0 | 0 | 0 |
| MOR2-1 | 0 | 0 | 0 | 0 | 0 | 0 |
| MOR203-1 | 0 | −3.039 | −3.907 | 0 | 0 | −4.001 |
| MOR204-6 | 0 | 0 | 0 | 0 | 0 | 0 |
| MOR205-1 | 0 | 0 | 0 | 0 | 0 | 0 |
| MOR207-1 | 0 | 0 | 0 | 0 | 0 | 0 |
| MOR222-1 | 0 | 0 | 0 | 0 | 0 | 0 |
| MOR223-1 | 0 | −3.487 | 0 | 0 | 0 | 0 |
| MOR23-1 | 0 | 0 | 0 | 0 | 0 | 0 |
| MOR236-1 | 0 | 0 | 0 | 0 | 0 | 0 |
| MOR25-1 | 0 | 0 | 0 | 0 | 0 | 0 |
| MOR250-1 | 0 | −3.545 | 0 | 0 | 0 | 0 |
| MOR251-1 | 0 | 0 | 0 | 0 | 0 | 0 |
| MOR253-1 | 0 | 0 | 0 | 0 | 0 | 0 |
| MOR256-17 | 0 | 0 | 0 | 0 | 0 | 0 |
| MOR258-1 | 0 | 0 | −3.099 | 0 | 0 | −3.38 |
| MOR259-1 | 0 | 0 | 0 | 0 | 0 | 0 |
| MOR260-1 | 0 | 0 | 0 | 0 | 0 | 0 |
| MOR261-1 | 0 | 0 | 0 | 0 | 0 | 0 |
| MOR268-1 | 0 | 0 | 0 | 0 | 0 | 0 |
| MOR269-1 | 0 | 0 | 0 | 0 | 0 | 0 |
| MOR271-1 | 0 | 0 | −5.033 | 0 | 0 | −3.18 |
| MOR272-1 | 0 | 0 | −5.098 | 0 | 0 | −3.272 |
| MOR273-1 | 0 | 0 | 0 | 0 | 0 | 0 |
| MOR277-1 | 0 | 0 | 0 | 0 | 0 | 0 |
| MOR30-1 | 0 | 0 | 0 | 0 | 0 | 0 |
| MOR31-1 | 0 | 0 | 0 | 0 | 0 | 0 |
| MOR33-1 | 0 | 0 | 0 | 0 | 0 | 0 |
| MOR37-1 | 0 | 0 | 0 | 0 | 0 | 0 |
| MOR4-1 | 0 | 0 | 0 | 0 | 0 | 0 |
| MOR40-1 | 0 | 0 | 0 | 0 | 0 | 0 |
| MOR41-1 | 0 | −4.143 | 0 | 0 | 0 | 0 |
| MOR5-1 | 0 | 0 | 0 | 0 | 0 | 0 |
| MOR9-1 | 0 | 0 | 0 | 0 | −6.334 | 0 |
| OR10J5 | 0 | 0 | 0 | 0 | 0 | 0 |
| OR1A1 | 0 | 0 | 0 | 0 | 0 | 0 |
| OR2C1 | 0 | 0 | 0 | 0 | 0 | 0 |
| OR2J2 | 0 | 0 | 0 | 0 | 0 | 0 |
| OR2M7 | 0 | 0 | 0 | 0 | 0 | 0 |
| OR2W1 | 0 | 0 | −4.071 | 0 | 0 | 0 |
| OR51E1 | 0 | 0 | 0 | 0 | 0 | 0 |
| OR51E2 | 0 | 0 | 0 | −3.963 | 0 | 0 |
| OR51L1 | 0 | 0 | 0 | 0 | 0 | 0 |
| OR5P3 | 0 | 0 | 0 | 0 | 0 | 0 |

[symbol]

indicates data missing or illegible when filed

TABLE 5

100-06-1
100-09-4
10022-28-3
10024-56-3
10024-57-4
10024-64-3
10024-97-2
1002-84-2
1003-04-9
10031-71-7
10031-82-0
10031-86-4
10031-87-5
10031-88-6
10031-90-0
10031-92-2
10031-93-3
10031-96-6
10032-00-5
10032-02-7
10032-05-0
10032-08-3
10032-13-0
10032-15-2
10039-39-1
100-42-5
100-51-6
100-52-7
100-53-8
10058-43-2
1006-27-5
100-66-3
100-68-5
10072-05-6
100-86-7
10094-34-5
10094-36-7
10094-41-4
101-39-3
101-41-7
101-48-4
101-49-5
10152-76-8
101-81-5
101-84-8
101-85-9
101-86-0
101-94-0
101-97-3
102-04-5
102-13-6
102-16-9
102-17-0
102-19-2
102-20-5
102-22-7
102369-06-2
102-69-2
102-76-1
103-05-9
103-07-1
103-13-9
10321-71-8
103-25-3
103-26-4
103-28-6
103-36-6
103-37-7
103-38-8
10339-55-6
10340-23-5
103-41-3
103-45-7
103-48-0
103-50-4
103-52-6
103-53-7
103-54-8
103-56-0
103-58-2
103-59-3
103-60-6
103-61-7
103-64-0
103694-68-4
103-82-2
103-93-5
103-95-7
10402-33-2
10402-47-8
10402-48-9
10402-52-5
104-09-6
10414-68-3
10415-87-9
10415-88-0
104-20-1
104-21-2
104-45-0
104-50-7
104-53-0
104-54-1
104-55-2
104-57-4
104-61-0
10461-98-0
104-62-1
104-64-3
104-65-4
104-67-6
104691-41-0
10471-14-4
10471-96-2
104-76-7
10482-55-0
10482-56-1
10482-77-6
10482-79-8
10484-09-0
10484-36-3
10486-14-3
10486-19-8
104-87-0
104-90-5
104-93-8
104986-28-9
105-01-1
105-13-5
10519-11-6
10519-33-2
105-21-5
10521-91-2
10522-18-6
105-37-3
105-43-1
10544-63-5
105463-44-3
105-53-3
105-54-4
105-57-7
105-60-2
105-66-8
105-68-0
105-79-3
10580-25-3
105-82-8
105-85-1
105-86-2
105-87-3
10588-10-0
10588-15-5
105-89-5
105-90-8
105-91-9
105-95-3
10599-70-9
106-02-5
106-18-3
106-21-8
106-22-9
106-23-0
106-24-1
106-25-2
106-26-3
106-27-4
106-28-5
106-29-6
106-30-9
106-32-1
106-33-2
106-35-4
106-36-5
106-44-5
106-65-0
106-68-3
106-70-7
106-72-9
106-73-0
107-03-9
107-10-8
1072-83-9
1073-11-6
1073-26-3
1073-29-6
107-35-7
107-41-5
107-43-7
1076-56-8
107-74-4
107-75-5
107-85-7
107-86-8
107-87-9
107898-54-4
1079-01-2
107-92-6
107-95-9
1080-12-2
108-10-1
108-21-4
108-22-5
108-39-4
108-47-4
108-48-5
108-50-9
108-64-5
108-82-7
108-83-8
108-94-1
108-95-2
108-98-5
109-05-7
109-08-0
109-15-9
109-19-3
109-20-6
109-21-7
109-25-1
109-29-5
109-42-2
109-52-4
109537-55-5
109-60-4
109-73-9
109-79-5
109-80-8
109-94-4
109-95-5
109-97-7
110-15-6
110-17-8
110-19-0
110-27-0
11031-45-1
110-38-3
110-39-4
110-40-7
110-41-8
110-42-9
110-43-0
110-44-1
110-45-2
110458-85-0
11050-62-7
110-58-7
110-62-3
110-66-7
110-74-7
110-81-6
110-85-0
110-86-1
110-89-4
110-93-0
110-98-5
111-11-5
111-12-6
111-13-7
111-14-8
111-26-2
111-27-3
111-28-4
111-30-8
1113-13-9
111-31-9
1113-21-9
1113-60-6
111-47-7
1115-11-3
111-66-0
111-70-6
111-71-7
1117-55-1
1117-61-9
111-79-5
111-80-8
111-81-9
111-82-0
1118-27-0
1118-39-4
111-87-5
111-90-0
1119-06-8
1119-44-4
112-05-0
112-06-1
112-12-9
112-14-1
112-17-4
112-19-6
112-23-2
1122-54-9
1122-62-9
112-30-1
112-31-2
112-32-3
112-37-8
1123-85-9
112-38-9
1124-11-4
112-42-5
112-43-6
112-44-7
112-45-8
1125-21-9
112-53-8
112-54-9
1125-88-8
112-63-0
112-66-3
112-80-1
1128-08-1
112-92-5
1129-47-1
1131-62-0
113486-29-6
113889-23-9
1139-30-6
1142-85-4
1153-51-1
115-95-7
115-99-1
116-02-9
116-26-7
116-53-0
117013-33-9
117933-89-8
117-98-6
1184-78-7
118-55-8
118-58-1
118-60-5
118-61-6
118-71-8
118-72-9
1188-02-9
1189-09-9
118-93-4
1191-16-8
1191-43-1
1191-62-4
1192-58-1
1192-62-7
1193-11-9
1193-18-6
119-36-8
1193-79-9
1195-32-0
119-53-9
1196-01-6
119-61-9
119-65-3
1197-01-9
119-84-6
1200-67-5
120-11-6
120-14-9
120-24-1
120-45-6
120-50-3
120-51-4
1205-17-0
120-57-0
120-72-9
120-92-3
1209-61-6
121-00-6
1211-29-6
121251-67-0
121251-68-1
121-32-4
121-33-5
121-34-6
121-39-1
121-44-8
121-79-9
121-98-2
122-00-9
122-03-2
1222-05-5
122-40-7
122-43-0
122-44-1
122-45-2
122-48-5
122-57-6
122-59-8
12262-03-2
122-63-4
122-67-8
122-68-9
122-69-0
122-70-3
122-72-5
122-73-6
122-74-7
122-78-1
122-79-2
122-84-9
122-91-8
122-97-4
122-99-6
123-07-9
123-08-0
123-11-5
123-15-9
123-19-3
123-25-1
123-29-5
123-32-0
123-35-3
123-38-6
123-51-3
123-54-6
123-63-7
123-66-0
123-68-2
123-72-8
123-75-1
123-76-2
123-86-4
123-96-6
124-04-9
124-06-1
124-07-2
124-10-7
124-12-9
124-13-0
124-19-6
124-25-4
124-76-5
125109-85-5
125-12-2
125352-06-9
126-64-7
126-91-0
127-17-3
127-41-3
127-42-4
127-43-5
127-51-5
127-91-3
128119-70-0
128-37-0
128-50-7
128-51-8
13002-09-0
13019-20-0
13019-22-2
13074-63-0
13074-65-2
13109-70-1
13112-65-7
13162-46-4
13171-00-1
131766-73-9
131812-67-4
13184-86-6
1319-88-6
1321-30-8
13215-88-8
1321-89-7
1322-17-4
1322-34-5
1322-58-3
1323-00-8
13246-52-1
13254-34-7
13257-44-8
133-18-6
1331-92-6
13327-56-5
1333-58-0
133-37-9
13341-72-5
1334-78-7
1334-82-3
13351-61-6
1335-44-0
1335-46-2
1335-66-6
13360-64-0
1337-83-3
134123-93-6
13419-69-7
134-20-3
134-28-1
13442-90-5
134454-31-2
13466-78-9
134769-33-8
13477-62-8
13481-87-3
13491-79-7
13494-06-9
13494-07-0
134-96-3
135-02-4
13532-18-8
13552-96-0
13567-39-0
135-79-5
13623-11-5
1365-19-1
136-60-7
13678-58-5
13678-59-6
13678-60-9
13678-67-6
13678-68-7
13679-46-4
13679-61-3
13679-70-4
13679-85-1
13679-86-2
136954-20-6
136954-21-7
136954-22-8
137-00-8
137-03-1
137-06-4
13706-86-0
13708-12-8
137-32-6
13794-15-5
13794-73-5
13816-33-6
138-22-7
138-23-8
13828-37-0
13851-11-1
13877-91-3
13877-93-5
138-86-3
138-87-4
13894-63-8
13925-00-3
13925-03-6
13925-05-8
13925-06-9
13925-07-0
139-45-7
139504-68-0
13952-84-6
139-70-8
13991-37-2
140-10-3
140-11-4
140-25-0
140-26-1
140-27-2
140-39-6
1405-92-1
140-67-0
14073-97-3
140-88-5
141-06-0
141-09-3
14109-72-9
141-10-6
141-11-7
141-12-8
141-13-9
141-14-0
141-15-1
141-16-2
141-25-3
14129-48-7
14159-61-6
14173-25-2
141-78-6
141-79-7
141-92-4
141-97-9
142-19-8
142-60-9
142-62-1
142-83-6
142-91-6
142-92-7
143-07-7
143-08-8
14309-57-0
143-13-5
143-14-6
143-28-2
14360-50-0
14371-10-9
14374-92-6
1438-91-1
1438-94-4
14400-67-0
14436-32-9
144-39-8
14481-52-8
14481-55-1
145-39-1
14576-08-0
14595-54-1
1460-34-0
14620-52-1
14667-55-1
1470-50-4
147159-49-7
14735-72-9
14765-30-1
147-85-3
14812-03-4
14901-07-6
149982-46-7
150-30-1
150436-68-3
1504-55-8
1504-74-1
1504-75-2
1506-02-1
150-60-7
150-78-7
150-84-5
150-86-7
151-05-3
151-10-0
15111-96-3
15111-97-4
151-19-9
1516-17-2
15186-51-3
15323-35-0
1534-08-3
15356-74-8
15373-31-6
154171-76-3
154171-77-4
1551-44-6
156-06-9
1565-76-0
1565-81-7
15679-12-6
15679-13-7
15679-19-3
1569-60-4
15706-73-7
15707-23-0
15707-24-1
1575-74-2
15760-18-6
15764-16-6
1576-77-8
1576-78-9
1576-85-8
1576-87-0
1576-95-0
1577-18-0
1577-19-1
15986-80-8
1599-47-9
1599-49-1
1604-28-0
1608-72-6
16128-68-0
1617-23-8
1617-40-9
1618-26-4
16251-77-7
1629-58-9
1632-73-1
16356-11-9
1639-09-4
16409-43-1
16409-45-3
16409-46-4
16429-21-3
1646-26-0
16491-24-0
16491-25-1
16491-36-4
16491-54-6
16493-80-4
1653-30-1
16587-71-6
16630-52-7
16630-55-0
16630-60-7
16630-66-3
166432-52-6
1669-44-9
1670-47-9
1679-07-8
169054-69-7
16930-93-1
16930-96-4
16939-73-4
1706-12-3
1708-34-5
1708-35-6
1708-81-2
17092-92-1
17102-64-6
1725-01-5
17283-81-7
1728-46-7
1731-84-6
1731-86-8
1733-25-1
17369-59-4
17369-60-7
17369-61-8
17373-84-1
17373-89-6
17488-65-2
17511-60-3
1754-62-7
17587-33-6
1759-28-0
17597-95-4
17619-36-2
17699-14-8
1775-43-5
1786-08-9
17909-77-2
1797-74-6
180031-78-1
18031-40-8
18060-79-2
18096-62-3
18114-49-3
181258-87-7
18127-01-0
18138-03-9
18138-04-0
18138-05-1
18172-67-3
18189-02-1
18189-07-6
182699-77-0
18277-27-5
18318-83-7
18368-91-7
18383-49-8
18402-83-0
18409-17-1
18409-18-2
18409-20-6
18409-21-7
18433-93-7
18436-37-8
18479-51-1
18479-54-4
18479-57-7
18479-58-8
18485-38-6
18492-65-4
18640-74-9
1866-31-5
18679-18-0
1871-67-6
1875-89-4
18776-92-6
1878-18-8
18794-77-9
18794-84-8
18824-63-0
18829-55-5
18829-56-6
18836-52-7
1883-78-9
1885-38-7
188570-78-7
188590-62-7
18871-14-2
1888-90-0
18999-28-5
19009-56-4
1901-26-4
1901-38-8
19089-92-0
19139-31-2
19141-40-3
19224-26-1
19269-28-4
19317-11-4
19322-27-1
19342-01-9
1946-00-5
19487-61-7
196109-18-9
1963-36-6
1968-40-7
19700-21-1
19788-49-9
19819-98-8
198404-98-7
19870-74-7
19872-52-7
19902-08-0
20053-88-7
20125-81-9
20125-84-2
2021-28-5
202188-43-0
20279-51-0
20286-45-7
20298-69-5
20333-39-5
2035-99-6
2040-10-0
20407-84-5
2044-73-7
2046-17-5
20474-93-5
20489-53-6
2049-96-9
53329
2050-08-0
53570
2050-87-5
2051-78-7
2051-96-9
2052-14-4
2052-15-5
20582-85-8
20662-84-4
20665-85-4
20680-10-8
20691-52-5
20777-49-5
20780-48-7
20780-49-8
20834-59-7
2084-18-6
2084-19-7
20920-83-6
2100-17-6
21016-46-6
21063-71-8
2109-22-0
2110-18-1
2111-75-3
21129-27-1
211323-05-6
2114-33-2
21145-77-7
21188-58-9
2120-70-9
21280-29-5
2142-94-1
2153-26-6
2153-28-8
21593-77-1
21661-97-2
21662-09-9
21662-13-5
21662-16-8
21722-83-8
2173-56-0
2173-57-1
2177-77-7
2179-57-9
2179-58-0
2179-59-1
2179-60-4
21834-92-4
21835-01-8
2186-92-7
21948-70-9
21963-26-8
21964-44-3
2198-61-0
22014-48-8
22029-76-1
22030-19-9
22047-25-2
22094-00-4
22104-80-9
22104-81-0
2216-45-7
2216-51-5
2216-52-6
2216-81-1
2217-33-6
117731
2235-83-8
22418-66-2
2244-16-8
22457-23-4
2253-73-8
129176
22629-49-8
22694-96-8
2270-60-2
227456-27-1
227456-28-2
22771-44-4
2277-16-9
2277-19-2
2278-53-7
22810-10-2
22882-89-9
2294-76-0
146589
2305-25-1
2306-78-7
2306-88-9
2306-91-4
2307-10-0
2308-18-1
2311-46-8
2315-68-6
23267-57-4
23328-62-3
23333-91-7
233665-96-8
2344-70-9
2345-24-6
2345-26-8
2345-28-0
162721
2349-13-5
23495-12-7
2351-90-8
23550-40-5
2363-88-4
23696-85-7
23726-91-2
23747-43-5
23747-45-7
23747-48-0
23787-80-6
23787-90-8
23811-08-7
23832-18-0
2396-77-2
2396-78-3
2396-83-0
2396-84-1
2403-58-9
24048-14-4
2408-20-0
2408-37-9
2412-80-8
24168-70-5
2416-94-6
24237-00-1
24295-03-2
2432-51-1
2432-77-1
2432-91-9
24330-52-7
2435-16-7
2437-25-4
2437-95-8
2439-44-3
24401-36-3
196780
2444-46-4
2444-49-7
2445-67-2
2445-72-9
2445-76-3
2445-77-4
2445-78-5
2463-53-8
24653-75-6
24680-50-0
24683-00-9
24691-15-4
24717-85-9
24720-09-0
24817-51-4
24851-98-7
2497-18-9
2497-21-4
2500-83-6
25013-16-5
2511-00-4
25152-84-5
25152-85-6
2520-60-7
25225-08-5
25225-10-9
25265-71-8
25279-09-8
228916
25304-14-7
25312-34-9
25409-08-9
25415-62-7
25415-67-2
25435-63-6
25485-88-5
2548-87-0
2550-11-0
2550-26-7
2550-40-5
2550-52-9
25524-95-2
238419
25564-22-1
240883
25634-93-9
2565-82-4
25680-57-3
25680-58-4
2568-25-4
25773-40-4
2623-23-6
2628-17-3
26303-90-2
2630-39-9
2639-63-6
2639-68-1
26446-31-1
26446-32-2
26553-47-9
26643-91-4
2679-87-0
26912-64-1
27039-84-5
2705-87-5
297890
2721-22-4
27538-09-6
2756-56-1
2758-18-1
27593-23-3
27606-09-3
27625-35-0
27743-70-0
27817-67-0
27829-72-7
2785-87-7
2785-89-9
27939-60-2
28043-10-9
28059-72-9
28069-74-1
28217-92-7
28219-60-5
28219-61-6
28231-03-0
2835-39-4
28371-99-5
2847-30-5
28588-73-0
28588-74-1
28588-75-2
28631-86-9
28645-51-4
28664-35-9
2882-20-4
28839-13-6
288-47-1
28897-58-7
28940-11-6
29021-37-2
290-37-9
29214-60-6
29350-73-0
29414-47-9
29460-90-0
29548-14-9
29548-30-9
29606-79-9
29725-66-4
29759-11-3
29811-50-5
2983-36-0
2983-37-1
2983-38-2
2986-54-1
29895-73-6
29896-45-5
29926-41-8
29926-42-9
3008-43-3
30086-02-3
30100-15-3
3016-19-1
30168-23-1
3025-30-7
302-72-7
30310-41-9
3033-23-6
30361-28-5
30361-29-6
30390-50-2
30408-61-8
30418-89-4
30453-31-7
3054-92-0
30640-46-1
30673-38-2
30772-79-3
30895-79-5
30897-75-7
3100-36-5
31375-17-4
313973-37-4
31416-78-1
3142-66-3
3142-72-1
3149-28-8
31499-71-5
31499-72-6
31501-11-8
31502-14-4
31539-84-1
3155-71-3
31704-80-0
3188-00-9
31906-04-4
3194-17-0
3208-16-0
3208-40-0
3209-13-0
32210-23-4
32214-91-8
32388-55-9
3239-35-8
3239-37-0
488211
3240-29-7
3249-68-1
32659-21-5
326-61-4
32665-23-9
3268-49-3
32736-91-7
32737-14-7
32764-98-0
32803-39-7
328-50-7
3289-28-9
3293-47-8
32974-92-8
3301-90-4
3301-94-8
33046-81-0
33049-93-3
3338-55-4
334-48-5
334-49-6
33467-73-1
33467-74-2
33467-76-4
33467-79-7
33522-69-9
33662-58-7
33673-62-0
33673-65-3
33704-61-9
3386-97-8
33885-51-7
543088
3391-83-1
3391-86-4
33922-73-5
34047-39-7
3407-42-9
34131-98-1
34135-85-8
34291-99-1
34300-94-2
34322-09-3
34365-79-2
34413-35-9
34451-19-9
34495-71-1
3452-97-9
34545-88-5
3460-44-4
34685-67-4
34686-71-0
34687-46-2
34764-02-8
3487-99-8
3488-00-4
3489-28-9
34902-57-3
350-03-8
35044-57-6
35044-58-7
35044-59-8
35087-49-1
3508-98-3
35154-45-1
35158-25-9
35178-55-3
35205-76-6
35206-51-0
35234-22-1
35250-53-4
352-93-2
35448-31-8
35472-56-1
3549-23-3
3558-60-9
3564-98-5
609379
3581-91-7
3583-00-4
35852-41-6
35852-46-1
35854-86-5
35926-04-6
3600-24-6
622683
3613-30-7
36219-73-5
36267-71-7
36306-87-3
36431-72-8
36438-54-7
3658-77-3
3658-79-5
3658-80-8
3658-93-3
3661-77-6
36653-82-4
36701-01-6
36789-59-0
36806-46-9
3681-71-8
3681-82-1
3682-42-6
37160-77-3
37172-02-4
37172-05-7
37172-53-5
3720-16-9
3724-61-6
3724-65-0
3738-00-9
37514-30-0
37526-88-8
3760-11-0
37609-25-9
37617-03-1
37674-63-8
37677-14-8
3777-69-3
3777-71-7
3782-00-1
37837-44-8
37887-04-0
3796-70-1
37973-51-6
38205-60-6
38205-64-0
38223-29-9
38285-49-3
38325-25-6
38363-23-4
707902
38433-74-8
38446-21-8
38462-22-5
38462-23-6
3848-24-6
3856-25-5
38713-41-6
38917-61-2
38917-62-3
39026-94-3
39067-39-5
39067-80-6
3913-71-1
3913-80-2
3913-81-3
3915-83-1
39212-23-2
39251-86-0
39251-88-2
39252-02-3
39252-03-4
39255-32-8
3943-74-6
39481-28-2
39711-79-0
39736-25-9
39741-41-8
39748-49-7
39770-05-3
39872-57-6
39900-38-4
39924-52-2
40010-99-9
40018-26-6
40188-41-8
40203-73-4
4023-65-8
40348-72-9
40527-42-2
40654-82-8
40716-66-3
4077-47-8
40785-62-4
40789-98-8
40790-04-3
40853-53-0
40853-56-3
40878-72-6
40910-49-4
40923-64-6
40942-73-2
4112-89-4
41199-19-3
41239-48-9
41270-80-8
41453-56-9
41496-43-9
41519-18-0
41519-23-7
41547-22-2
41653-97-8
4166-20-5
41724-19-0
4180-23-8
41816-03-9
41820-22-8
41847-88-5
41890-92-0
42075-45-6
42078-65-9
4208-49-5
4208-57-5
4230-97-1
42348-12-9
42370-07-0
42436-07-7
42474-44-2
4253-89-8
42604-12-6
426218-78-2
4265-16-1
4265-97-8
42822-86-6
42824-62-4
42919-64-2
43039-98-1
43040-01-3
431-03-8
4312-99-6
879933
43219-68-7
432-25-7
894025
4351-54-6
4359-31-3
4359-47-1
4359-57-3
4360-47-8
4361-23-3
4362-22-5
898649
4395-92-0
4410-99-5
4411-89-6
4426-79-3
4430-31-3
4430-36-8
4430-39-1
4430-42-6
4433-36-7
4437-20-1
4437-22-3
4437-51-8
443-79-8
4440-65-7
4442-79-9
4455-13-4
4466-24-4
4493-42-9
4500-58-7
4501-58-0
45019-28-1
4532-64-3
4536-23-6
4573-50-6
4586-22-5
459-80-3
4602-84-0
4606-15-9
992463
995835
4630-82-4
4634-89-3
4643-25-8
4643-27-0
464-43-7
464-45-9
464-49-3
465-24-7
4674-50-4
4695-62-9
469-61-4
470-67-7
4707-47-5
470-82-6
472-66-2
4728-82-9
472-97-9
473-15-4
4732-13-2
473-67-6
4740-79-8
1038568
4748-78-1
475-03-6
475-20-7
4798-44-1
4798-61-2
4819-67-4
4826-62-4
4861-58-9
4861-85-2
4864-61-3
488-10-8
4883-60-7
489-40-7
489-86-1
490-03-9
4906-24-5
491-04-3
491-07-6
491-09-8
4927-39-3
4938-52-7
1109189
4948-28-1
494-90-6
495-40-9
495-62-5
495-76-1
49576-57-0
496-77-5
497-03-0
497-23-4
49776-81-0
498-00-0
498-02-2
498-81-7
499-12-7
499-44-5
499-54-7
499-70-7
499-75-2
500-02-7
5009-32-5
501-52-0
501-92-8
50-21-5
502-47-6
502-61-4
502-69-2
502-72-7
50373-36-9
503-74-2
505-10-2
505-29-3
505-32-8
505-57-7
505-79-3
50607-64-2
50623-57-9
50626-02-3
50-69-1
50-70-4
507-09-5
50746-10-6
507-70-0
5077-67-8
50816-18-7
50-81-7
5090-41-5
51100-54-0
51115-67-4
51154-96-2
512-13-0
5132-75-2
513-44-0
51352-68-2
5137-52-0
513-86-0
5146-66-7
515-00-4
51532-26-4
51534-66-8
51566-62-2
515-69-5
51595-91-6
516-06-3
51608-18-5
51652-47-2
5166-53-0
51-67-2
51755-66-9
51755-83-0
51755-85-2
5182-36-5
1205849
1205978
52210-18-1
5240-32-4
52474-60-9
52517-67-6
52557-97-8
5271-38-5
527-60-6
52789-73-8
5287-45-6
52-90-4
5292-21-7
53018-24-9
53046-97-2
53053-51-3
531-26-0
531-59-9
5320-75-2
53243-59-7
53243-60-0
53263-58-4
5328-37-0
5331-14-6
5331-32-8
533-18-6
53338-05-9
53398-78-0
53398-80-4
53398-83-7
53398-85-9
53398-86-0
53398-87-1
53399-81-8
53405-97-3
534-15-6
534-22-5
53448-07-0
53475-15-3
53488-14-5
5349-62-2
53498-32-1
5362-56-1
536-50-5
536-59-4
536-60-7
536-78-7
53767-93-4
53833-30-0
53833-32-2
538-65-8
538-85-3
53889-39-7
5392-40-5
539-30-0
5396-89-4
539-82-2
539-88-8
539-90-2
540-07-8
540-18-1
540-42-1
5405-41-4
5405-58-3
1279415
540-63-6
5406-58-6
54082-68-7
54089-83-7
541-31-1
541-35-5
5413-60-5
541-47-9
541-58-2
5418-86-0
541-91-3
1284897
5421-17-0
5421-27-2
5422-34-4
542-46-1
542-55-2
5426-78-8
542-85-8
54300-08-2
54300-09-3
543-39-5
54340-90-8
5434-57-1
543-49-7
5435-64-3
5436-21-5
54393-36-1
54411-06-2
5442-00-2
544-40-1
54440-17-4
544-63-8
54464-57-2
54464-59-4
54484-73-0
5451-52-5
5451-80-9
5451-88-7
5451-95-6
1296065
1296855
1296920
5454-19-3
5454-21-7
5454-28-4
54546-26-8
5457-70-5
5458-59-3
1299385
1299690
54644-28-9
546-49-6
1301153
546-79-2
1301880
5471-51-2
547-63-7
54814-64-1
54815-13-3
54830-99-8
548774-80-7
54889-48-4
54947-74-9
54957-02-7
54982-83-1
55066-48-3
55066-49-4
55066-56-3
551-08-6
551-93-9
55253-28-6
554-12-1
55418-52-5
5552-30-7
5555-90-8
556-24-1
556-61-6
556-82-1
557-00-6
55704-78-4
55719-85-2
557-48-2
55764-22-2
55764-23-3
55764-25-5
55764-28-8
5577-44-6
5579-78-2
5595-79-9
56011-02-0
560-88-3
56-12-2
5616-51-3
56172-46-4
562-74-3
56-40-6
564-20-5
564-94-3
5655-61-8
5660-60-6
56700-78-8
56747-96-7
56767-18-1
56805-23-3
56-81-5
56836-93-2
56-84-8
56-85-9
56-86-0
56-87-1
56922-74-8
5694-72-4
56973-85-4
57-06-7
57074-37-0
57082-24-3
57094-40-3
57-10-3
57-11-4
57124-87-5
5724-81-2
57345-19-4
57378-68-4
57500-00-2
57-55-6
5756-24-1
57568-60-2
57576-09-7
5760-50-9
576-26-1
5764-85-2
577-16-2
57743-63-2
578-58-5
57893-27-3
579-07-7
57934-97-1
1420859
1420891
579-74-8
579-75-9
579-93-1
58-08-2
58214-97-4
58296-81-4
583-04-0
583-60-8
5837-78-5
583-92-6
58430-94-7
585-25-1
58567-11-6
585-84-2
58625-96-0
586-38-9
586-62-9
586-81-2
586-82-3
5870-58-8
5870-93-9
58-86-6
588-67-0
589-35-5
58936-30-4
589-38-8
589-59-3
589-66-2
589-75-3
589-82-2
58985-18-5
589-92-4
589-98-0
590-01-2
59020-85-8
59020-90-5
59021-02-2
59052-82-3
5905-46-4
5905-47-5
59056-62-1
59056-64-3
59056-70-1
590-86-3
5910-87-2
5910-89-4
591-12-8
591-24-2
591-49-1
59151-19-8
591-60-6
591-68-4
591-80-0
591-82-2
59191-78-5
592-20-1
59230-57-8
5925-68-8
5925-75-7
59259-38-0
592-82-5
592-84-7
592-88-1
593-08-8
59323-76-1
59324-17-3
59354-71-1
59376-58-8
5943-34-0
59471-80-6
5947-36-4
1477529
59-51-8
59558-23-5
5986-38-9
598-75-4
5988-91-0
5989-27-5
5989-54-8
600-14-6
600-18-0
60047-17-8
60-12-8
60-18-4
60241-53-4
60241-55-6
6028-61-1
6032-29-7
60-33-3
60335-71-9
60-35-5
60415-61-4
60523-21-9
606-45-1
6066-49-5
60763-41-9
60788-25-2
607-90-9
60-82-2
60826-15-5
6091-50-5
611-13-2
61114-24-7
61197-09-9
612-15-7
61295-41-8
61295-44-1
61295-50-9
61295-51-0
613-70-7
61444-38-0
61444-41-5
614-99-3
615-10-1
616-25-1
61692-83-9
61692-84-0
61699-38-5
617-01-6
617-35-6
617-50-5
6175-49-1
61792-11-8
61810-55-7
61826-53-7
619-01-2
61-90-5
6191-71-5
61920-45-4
61931-80-4
61931-81-5
620-02-0
620-23-5
620-79-1
62079-29-2
62147-49-3
621-82-9
6221-93-8
6222-35-1
622-39-9
622-45-7
622-62-8
622-78-6
623-05-2
623-15-4
623-17-6
623-19-8
623-21-2
623-22-3
623-30-3
623-36-9
623-37-0
623-42-7
623-70-1
62395-45-3
624-09-9
624-24-8
62439-41-2
624-41-9
624-51-1
624-54-4
62488-56-6
624-89-5
624-92-0
625-33-2
625-55-8
625-60-5
62563-80-8
625-80-9
6258-63-5
625-86-5
6259-76-3
626-11-9
6263-65-6
626-38-0
626-77-7
626-82-4
1594715
6270-56-0
627-90-7
628-00-2
628-03-5
6281-40-9
6284-46-4
628-46-6
628-63-7
628-97-7
628-99-9
6290-17-1
6290-37-5
629-12-9
629-19-6
629-33-4
629-45-8
629-59-4
629-62-9
629-63-0
629-70-9
6297-41-2
6304-24-1
6309-51-9
6314-97-2
63187-91-7
1616205
63449-64-9
63449-68-3
63500-71-0
635-46-1
63759-55-7
637-64-9
637-65-0
63767-86-2
637-78-5
6378-65-0
6380-23-0
638-02-8
638-11-9
638-17-5
638-25-5
638-49-3
638-53-9
63-91-2
63986-03-8
64001-15-6
64-04-0
1647153
6413-26-9
64165-57-7
64-17-5
64-18-6
64187-83-3
64-19-7
64275-73-6
644-08-6
644-13-3
644-35-9
644-49-5
64461-99-0
645-13-6
6454-22-4
645-56-7
6457-30-3
64577-91-9
646-07-1
6485-40-1
6493-80-7
65113-95-3
65113-99-7
1683469
65155-45-5
65330-49-6
65405-67-6
65405-68-7
65405-70-1
65405-72-3
65405-73-4
65405-76-7
65405-77-8
65405-80-3
65405-84-7
65416-14-0
65423-25-8
65442-31-1
65443-14-3
65504-97-4
65505-16-0
65505-17-1
65505-18-2
65505-24-0
65505-25-1
65530-53-2
65588-69-4
6561-39-3
65620-50-0
656-53-1
65737-52-2
65813-53-8
65817-24-5
65-85-0
65887-08-3
65894-82-8
65894-83-9
659-70-1
66062-78-0
66068-84-6
66072-32-0
6622-76-0
6624-71-1
66-25-1
6627-88-9
6628-18-8
66327-54-6
6635-22-9
1729185
66408-78-4
66576-71-4
6658-48-6
66634-97-7
66642-86-2
66848-40-6
67028-40-4
6707-60-4
6725-64-0
6728-26-3
6728-31-0
67355-38-8
6738-23-4
67452-27-1
675-09-2
6753-98-6
67601-05-2
67-63-0
67633-94-7
67633-96-9
67633-97-0
67633-99-2
67634-00-8
67634-01-9
67634-07-5
67634-11-1
67634-14-4
67634-15-5
67634-17-7
67634-20-2
67634-22-4
67634-23-5
67634-25-7
67534-26-8
67-54-1
67662-96-8
67663-01-6
67674-36-6
67674-46-8
67-68-5
67689-50-3
67707-75-9
67715-79-1
67715-80-4
67739-11-1
67746-30-9
67770-79-0
67785-76-6
67785-77-7
67801-20-1
67801-33-6
67801-38-1
67801-44-9
67801-47-2
67801-64-3
67801-65-4
6784-13-0
67845-30-1
67845-42-5
67845-46-9
67859-96-5
67860-38-2
67874-67-3
67874-69-5
67874-72-0
67874-78-6
67874-81-1
67879-60-1
67883-79-8
6789-80-6
6789-88-4
67905-40-2
6790-58-5
67919-67-9
67920-63-2
67952-59-4
67952-60-7
67952-65-2
67999-56-8
6803-40-3
68039-24-7
68039-26-9
68039-29-2
68039-39-4
68039-44-1
68039-47-4
68039-49-6
68039-69-0
68039-73-6
68083-58-9
68084-03-7
68084-04-8
68127-22-0
68132-80-9
68133-72-2
68133-73-3
68133-75-5
68133-76-6
68133-77-7
68133-78-8
68133-79-9
68140-48-7
68141-17-3
68155-66-8
68155-67-9
68213-87-6
68258-95-7
68259-33-6
68345-22-2
68378-13-2
68391-29-7
68391-39-9
68398-18-5
68411-38-1
68419-46-5
68480-06-8
68480-08-0
68480-11-5
68480-14-8
68480-15-9
68480-25-1
68480-26-2
68480-27-3
68480-28-4
68527-74-2
68527-76-4
68527-77-5
68555-53-3
68555-57-7
68555-58-8
68555-59-9
68555-61-3
68555-62-4
68555-63-5
68555-65-7
68555-94-2
68555-95-3
68683-20-5
68683-25-0
68698-57-7
68698-59-9
68705-63-5
68738-94-3
68738-96-5
68738-99-8
68739-00-4
6876-13-7
68845-00-1
68845-02-3
68845-36-3
68877-29-2
688-82-4
68901-15-5
68901-22-4
68901-32-6
68922-10-1
68922-11-2
68928-82-5
68959-28-4
68966-86-9
689-67-8
689-89-4
68991-97-9
69038-78-4
69103-20-4
6911-51-9
6915-15-7
6920-22-5
69226-05-7
692-86-4
69300-15-8
6931-54-0
693-54-9
69382-62-3
693-95-8
69486-14-2
695-06-7
6963-56-0
69668-87-7
69-72-7
6975-60-6
6976-72-3
698-10-2
698-27-1
6986-51-2
698-76-0
69882-09-3
699-10-5
699-17-2
69925-33-3
69929-16-4
69929-17-5
70092-23-8
7011-83-8
70214-77-6
705-73-7
705-86-2
706-14-9
7070-15-7
707-29-9
70788-30-6
70851-60-4
70851-61-5
71-00-1
710-04-3
71048-82-3
71077-31-1
71159-90-5
71-23-8
71298-42-5
71-36-3
713-95-1
71-41-0
7143-69-3
7149-26-0
7149-29-3
7149-32-8
71500-37-3
1918228
71566-51-3
71566-53-5
71648-34-5
71660-03-2
71735-79-0
71832-76-3
72007-81-9
72089-08-8
72117-72-7
7212-44-4
7217-59-6
72-18-4
72214-23-4
72257-53-5
72403-67-9
72429-08-4
72437-56-0
72437-68-4
7251-61-8
72797-17-2
72854-42-3
72881-27-7
72894-12-3
7289-52-3
72928-51-9
72928-52-0
73003-91-5
1973383
73157-43-4
7335-26-4
7341-17-5
73545-18-3
73545-19-4
7351-80-0
7367-81-9
7367-82-0
7367-88-6
73757-27-4
7392-19-0
7403-42-1
74094-60-3
74094-61-4
74094-62-5
74094-63-6
7416-35-5
74338-72-0
74356-31-3
74367-97-8
74410-10-9
7452-79-1
7460-74-4
74758-91-1
74758-93-3
74-79-3
7492-37-7
7492-39-9
7492-41-3
7492-44-6
7492-45-7
7492-65-1
7492-66-2
7492-67-3
7492-69-5
7492-70-8
74-93-1
7493-57-4
7493-58-5
7493-59-6
7493-63-2
7493-65-4
7493-66-5
7493-68-7
7493-69-8
7493-71-2
7493-72-3
7493-74-5
7493-76-7
7493-78-9
7493-79-0
7493-80-3
7493-82-5
75-04-7
75048-15-6
75-07-0
75-08-1
75147-23-8
75-18-3
75-21-8
75-31-0
2056136
75-33-2
7540-51-4
7540-53-6
7541-49-3
7549-33-9
7549-37-3
7549-41-9
75-50-3
2063957
75853-49-5
759-05-7
76-09-5
762-26-5
762-29-8
76238-22-7
764-39-6
764-40-9
76-49-3
765-05-9
76-50-6
765-70-8
7664-38-2
76649-14-4
76649-16-6
76649-17-7
76649-22-4
76649-23-5
76649-26-8
766-92-7
76788-46-0
770-27-4
77118-93-5
77311-02-5
7732-18-5
774-55-0
77-53-2
77-54-3
7756-96-9
7764-50-3
77-70-3
7774-44-9
7774-47-2
7774-60-9
7774-65-4
77-74-7
7774-74-5
7774-79-0
7774-82-5
7774-96-1
7775-00-0
7775-38-4
7775-39-5
7778-83-8
7778-87-2
7778-96-3
7779-16-0
7779-17-1
7779-23-9
7779-30-8
7779-41-1
7779-50-2
7779-65-9
7779-70-6
7779-72-8
7779-73-9
7779-75-1
7779-77-3
7779-78-4
7779-80-8
7779-81-9
7779-94-4
2146322
77-83-8
7784-67-0
7784-98-7
77851-07-1
7785-33-3
7785-53-7
7785-53-9
7785-66-2
7786-29-0
7786-47-2
7786-48-3
7786-58-5
7786-61-0
7787-20-4
77-92-9
77-93-0
78-35-3
78-36-4
78-37-5
78417-28-4
78-59-1
78649-62-4
78-69-3
78-70-8
78-81-9
78-83-1
78-84-2
78-93-3
78-96-6
78-98-8
78989-37-4
79-09-4
79-20-0
79-31-2
79-42-5
79-69-6
79771-15-8
79-77-6
79-78-7
79-89-0
79915-74-5
79-92-5
8007-35-0
80118-08-5
80-25-1
80-26-2
80-27-3
80417-97-6
80449-58-7
80480-24-6
80-54-6
80-56-8
80-57-9
80-59-1
80823-07-0
80-62-6
80857-64-3
80-71-7
80858-47-5
80866-83-7
81-14-1
81-15-2
814-67-5
816-68-0
81782-87-6
81782-77-6
81783-01-9
81786-73-4
81925-81-7
821-41-0
821-55-6
82 [symbol] 85-41-9
823-22-3
82358-51-2
82481-14-1
825-51-4
82854-98-5
82784-84-7
828-26-2
83-34-1
83-66-9
83-67-0
84012-84-8
84029-92-5
84029-93-8
84434-18-4
84842-60-4
84842-61-5
84-88-2
84881-92-5
84897-09- [symbol]
84788-08- [symbol]
85136-08-7
85213-22-5
85351-07-1
85392-03-8
85508-08-3
85554-72-9
85568-87-0
85761-70-2
85-91-8
88241-90-9
88803-90-9
868-57-5
870-23-5
87081-04-9
87118-95-4
87-19-4
87-20-7
87-22-9
87-25-2
87-29-6
87-41-2
87-44-5
874-88-8
874-90-8
87641-23-4
87841-24-5
87-89-4
87731-18-8
87-91-2
88-09-5
88-15-3
881-68-5
882-33-7
88-29-9
88-41-5
88-59-7
88-84-8
89-47-4
89-48-5
89534-38-3
89-65-6
89-74-7
89780-08-3
89-78-1
89-79-2
89-81-6
89-82-7
89-83-8
89-88-1
89-88-3
90-02-8
9003-73-0
90-05-1
90-12-0
90-17-5
90397-38-9
90-42-6
90-43-7
90530-04-4
90-87-9
91009-40-8
91-10-1
91-16-7
91-22-5
91482-37-0
91-60-1
91-61-2
91-62-3
91-64-5
91-87-2
92048-48-5
92-48-8
92-52-4
925-78-0
92585-24-5
927-49-1
928-80-3
928-91-5
928-92-7
928-94-9
928-95-0
928-96-1
928-97-2
93-04-9
93-08-3
93-15-2
93-18-3
93-18-5
93-19-5
932-16-1
93-28-7
93-29-8
93-51-6
93-53-8
93-54-9
93-55-0
93-58-3
93-80-7
937-30-4
93762-35-7
93840-90-5
93-89-0
93905-03-4
93-91-4
939-21-9
93-92-5
93939-86-7
939-48-0
93952-58-0
93981-50-1
94-02-0
94021-42-8
94087-23-7
94087-83-9
94089-01-7
94089-02-8
94-13-3
94134-03-9
94159-31-6
94159-32-7
941-98-0
94201-19-1
94201-73-7
94-26-8
94278-27-0
94293-57-9
94-30-4
94346-09-5
94386-48-8
94405-15-2
94-46-2
94-47-3
94-48-4
94-50-8
94-62-2
94-85-0
950-33-4
95-16-9
95-21-8
95-41-0
95-48-7
95-85-8
95-87-4
959 [symbol] 2-14-4
9 [symbol] -04-8
9 [symbol] -15-1
96-17-3
96-28-4
96-48-0
96-54-8
97358-54-8
97358-55-9
97384-48-0
97-41-6
97-42-7
97-45-0
97-53-0
97-54-1
97-61-0
97-62-1
97-64-3
97-87-8
97752-28-8
97-85-8
97-87-0
97890-13-6
97-89-2
97-98-1
97-99-4
98-00-0
98-01-1
98-02-2
98-52-2
98-53-3
98-54-4
98-55-5
98-85-1
98-85-2
98-89-5
99-48-9
99583-29-6
996-97-4
99-72-9
99-76-3
99-83-2
99-85-4
99-86-5
99-87-6
99-93-4
999-40-6
99-96-7
300-57-2
391-78-6
1455-21-6
94805-33-1
938-79-4
4288-15-1
1076-38-6
130086-44-3
484-48-2
491-37-2
5324-05-0
553-86-6
71-43-2 [symbol]
-11-1
123-92-2
123-99-9
126-81-8
6033-23-4
6169-08-8
20487-40-5 [symbol]
indicates data missing or illegible when filed

TABLE 6

| | Position in | |
|---|---|---|
| Property | Alignment | Weight |
| Volume | 24 | 1 |
| Volume | 31 | 1 |
| Composition | 43 | 1 |
| Volume | 52 | 1 |
| Volume | 71 | 1 |
| Composition | 56 | 1 |
| Polarity | 107 | 1 |
| Composition | 140 | 1 |
| Polarity | 149 | 1 |
| Volume | 160 | 1 |
| Polarity | 171 | 1 |
| Polarity | 210 | 1 |
| Volume | 255 | 2 |
| Composition | 257 | 1 |
| Composition | 267 | 2 |
| Polarity | 276 | 2 |

### Example 2

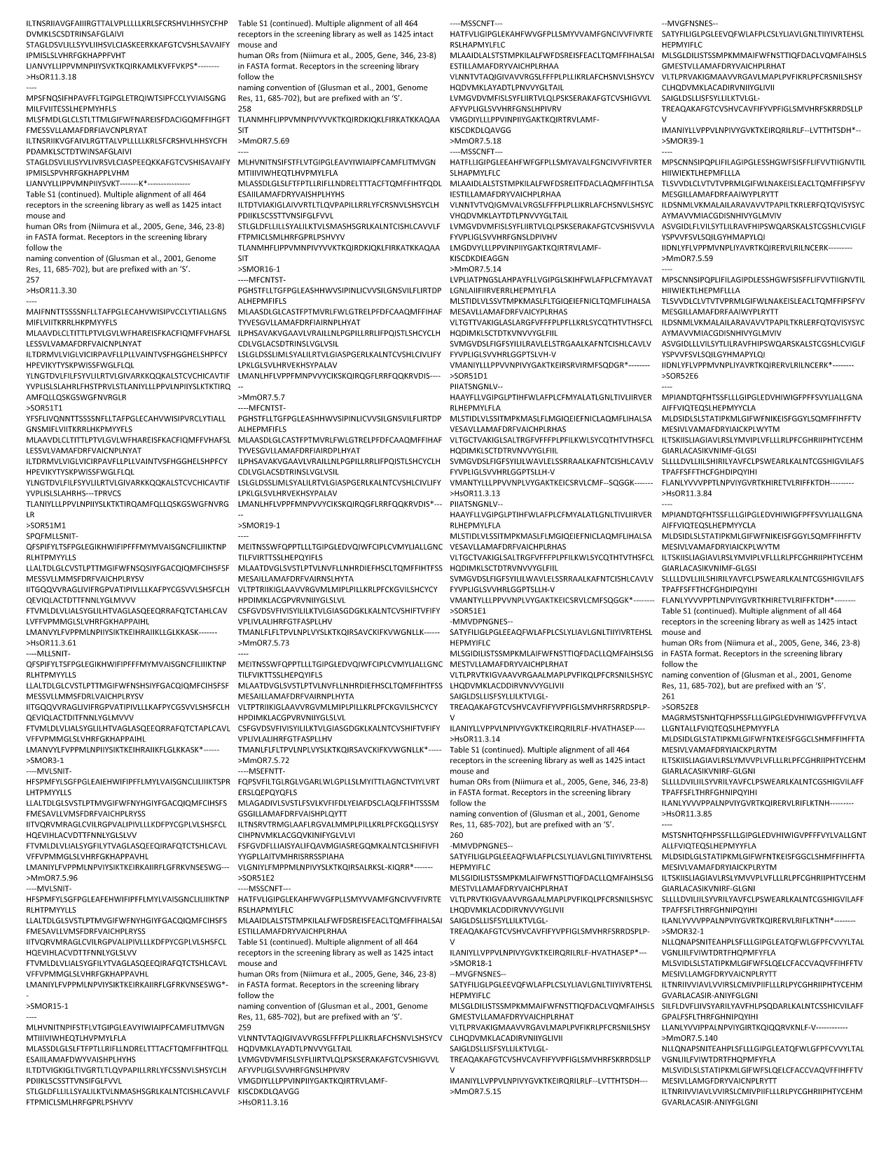

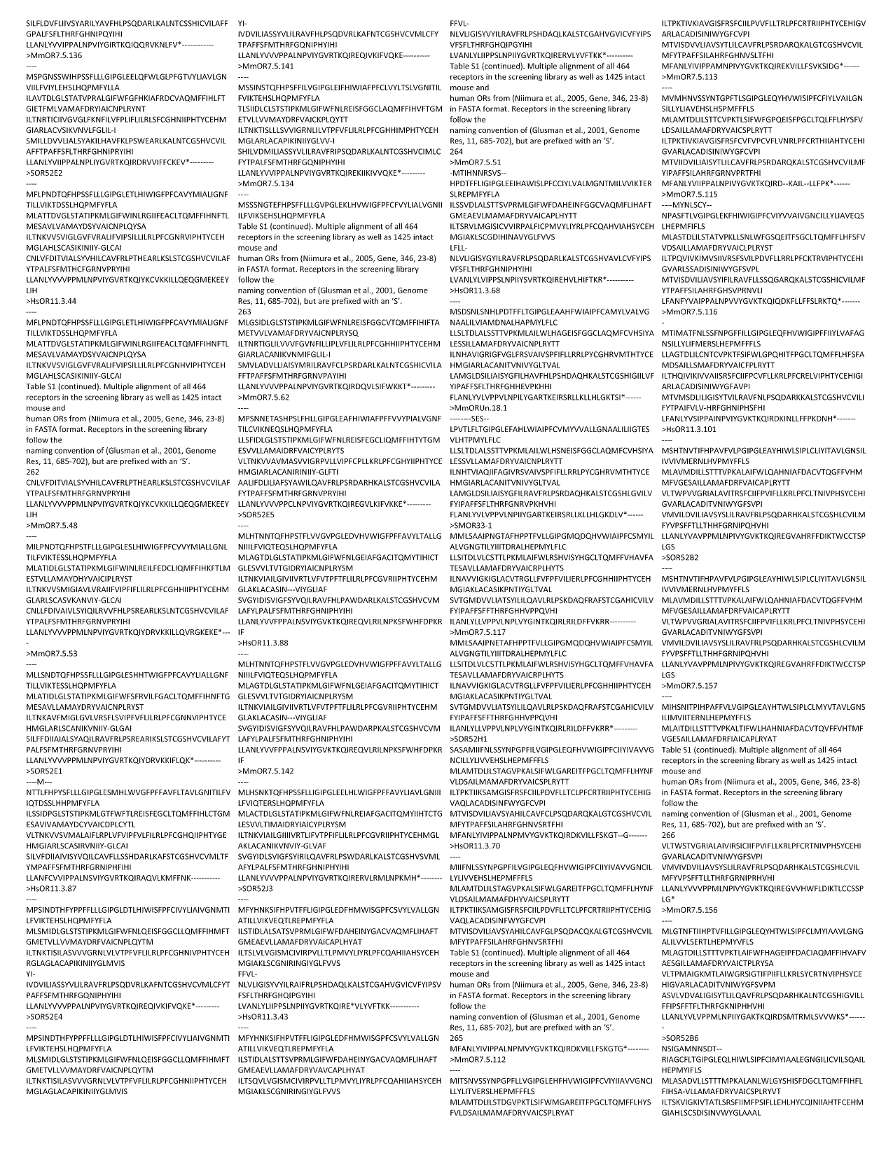

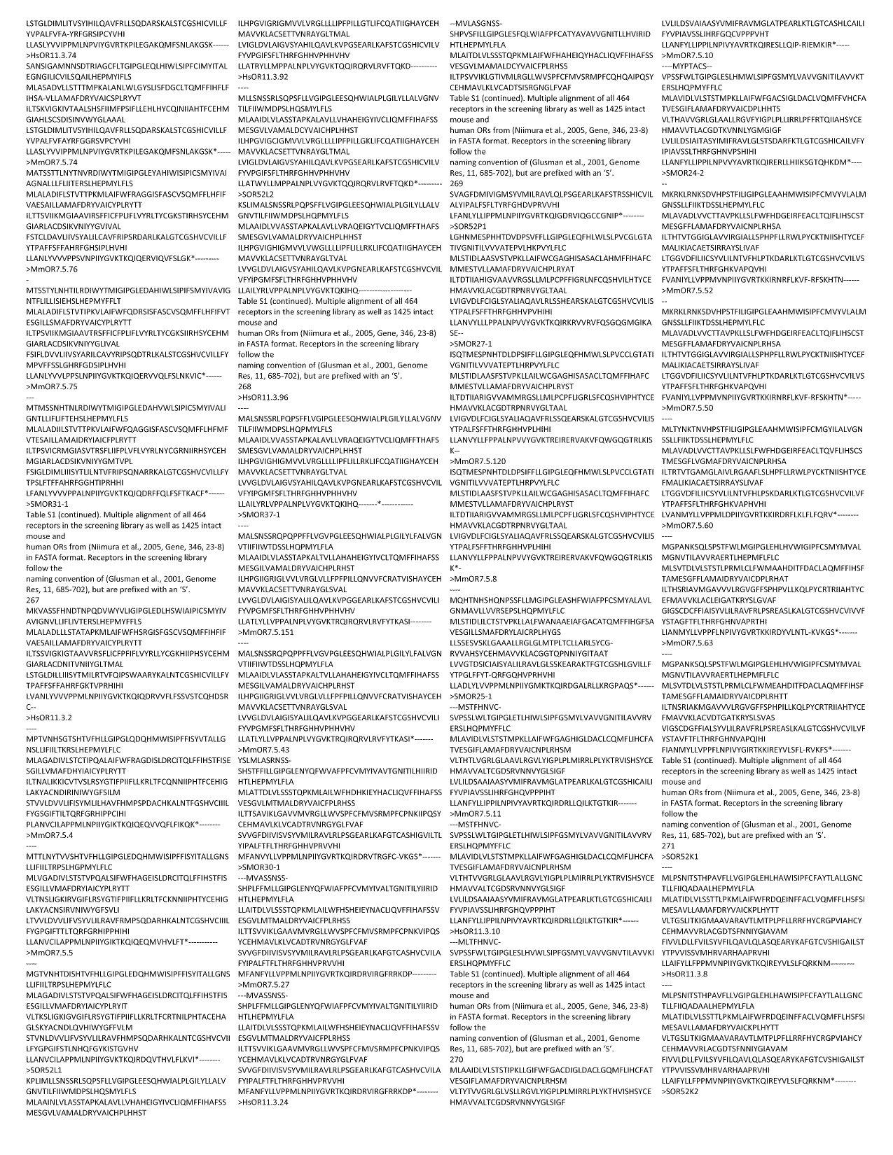

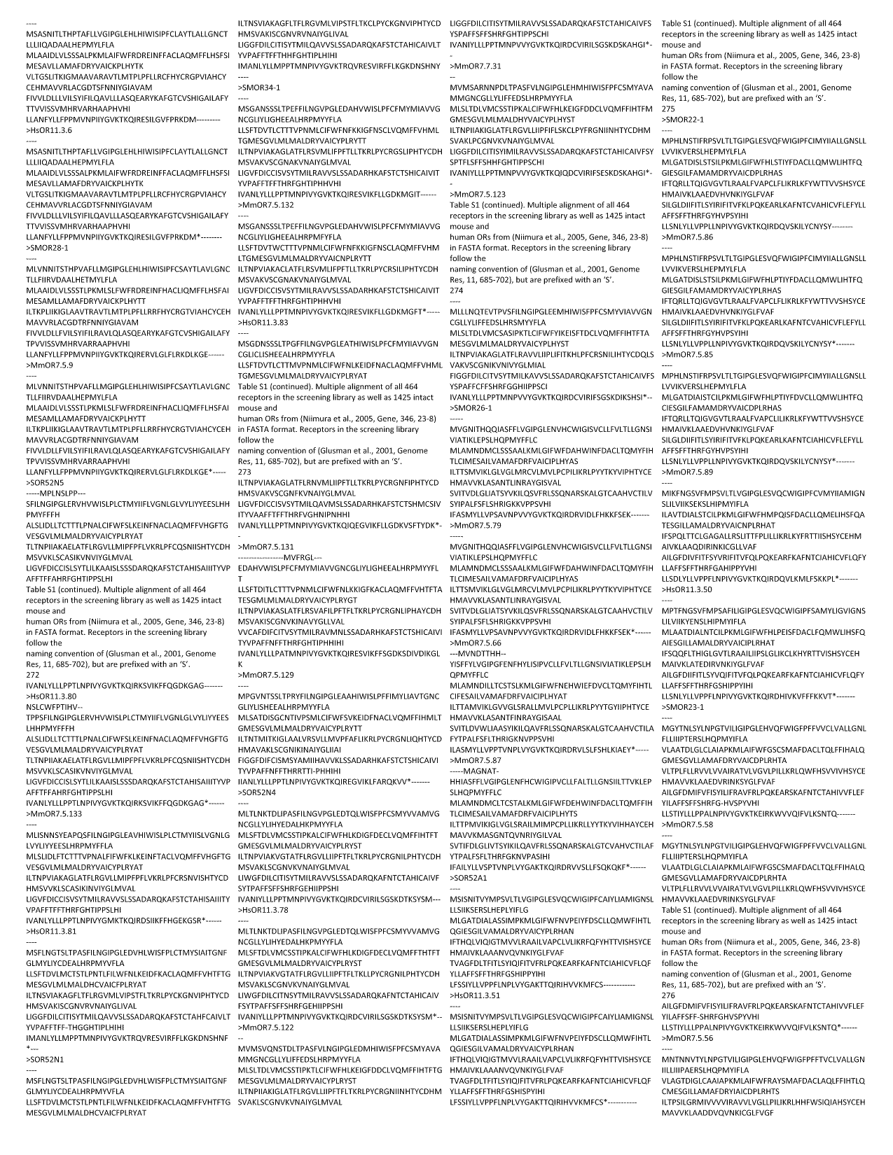

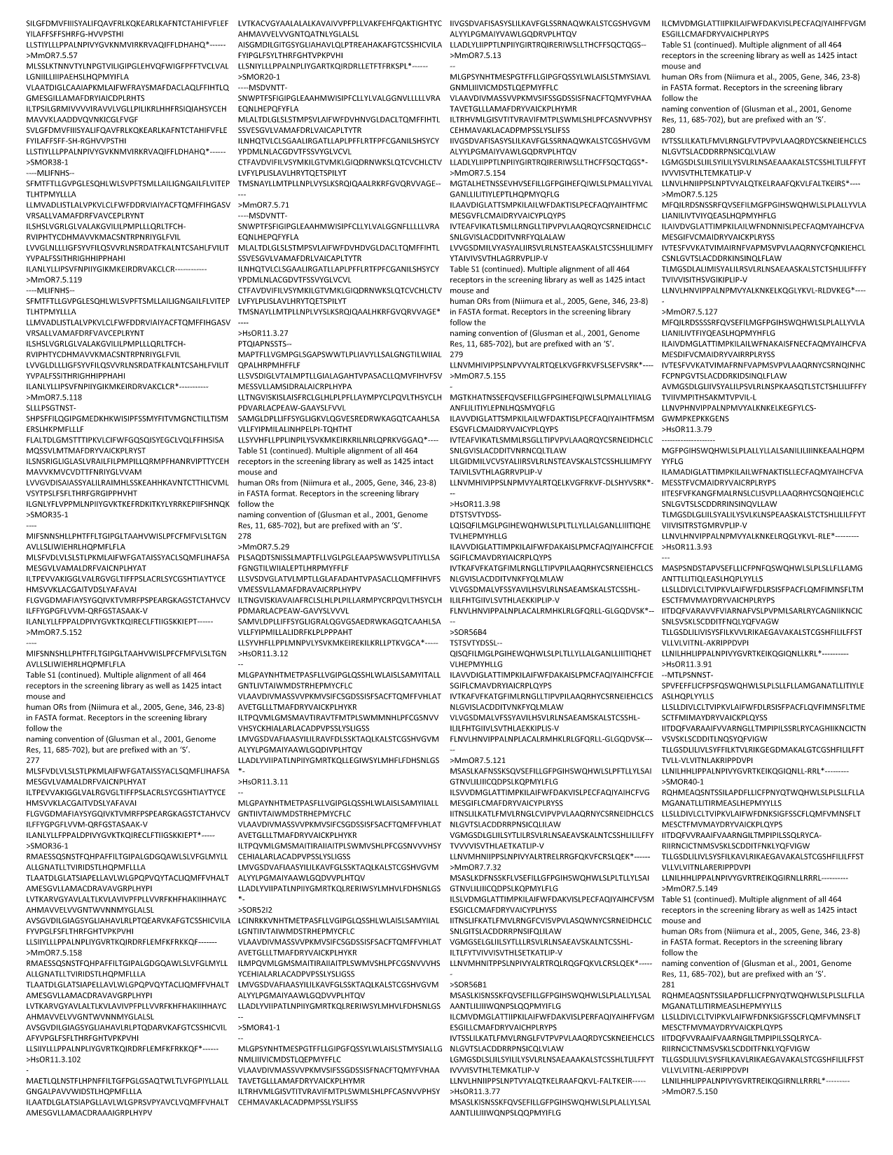

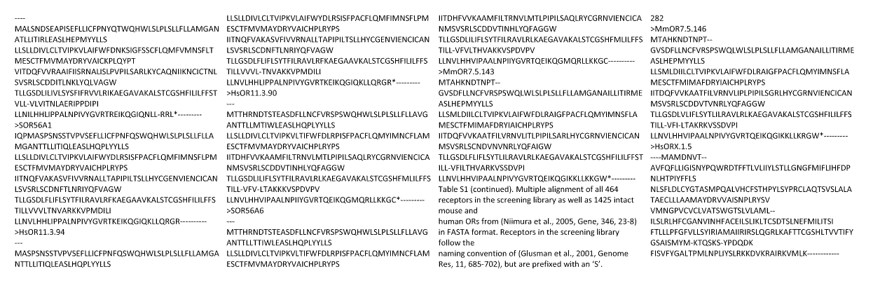

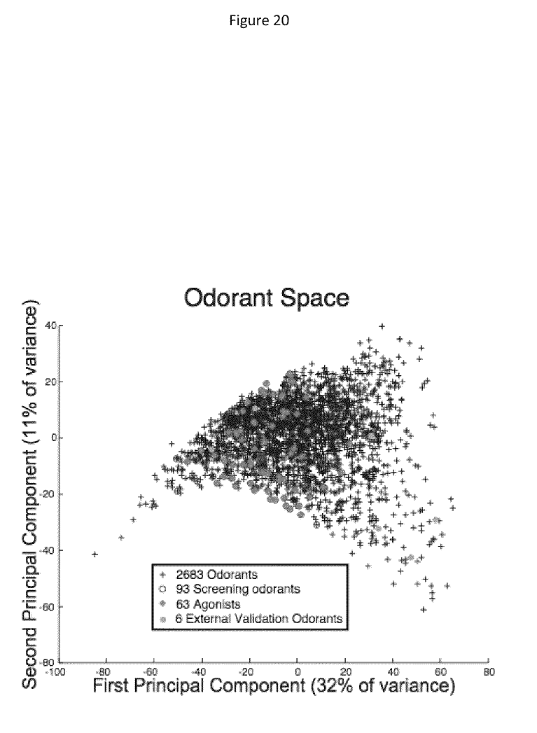

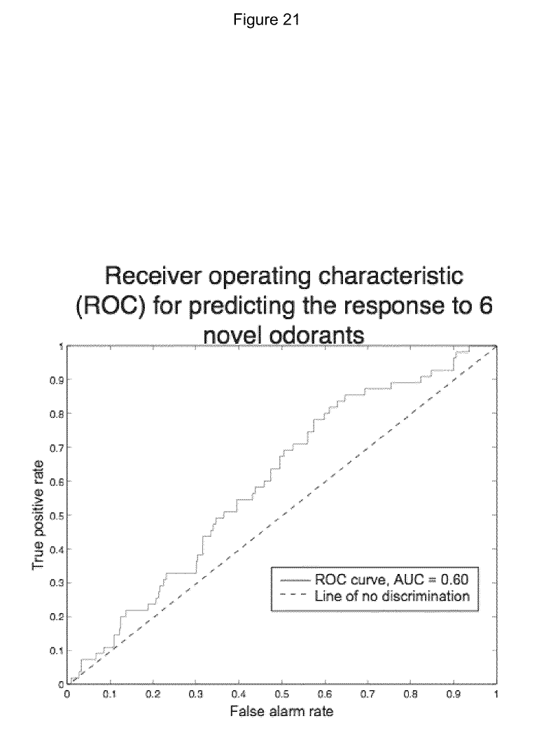

Six odorants (FIG. 20) were tested at 6 concentrations (300 μM, 100 μM, 33 μM, 11 μM, 3.7 μM, and no odor) against the 62 receptors from data set described herein. The odorants used were:

### Quinoline (91-22-5)

1,3-butanedithiol (24330-52-7)

### Pentadecalactone (106-02-5)

Geranyl linalool (113-21-9)
Hexyl octanoate (1117-55-1)
Octyl octanoate (2306-88-9)

The EC₅₀ for all 62×6 combinations was estimated as described previously. The odorant receptor model outputs the probability that an odorant activates a receptor. Using the new dataset described herein, the model has an AUC of 0.6.

### INCORPORATION BY REFERENCE

The entire disclosure of each of the patent documents and scientific articles referred to herein is incorporated by reference for all purposes.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method of identifying an odorant ligand for an odorant receptor, comprising:
a) comparing three or more amino acid property descriptors of an odorant receptor with three or more physicochemical properties of a plurality of odorants; and
b) identifying one or more odorant receptor for said odorant receptor based on said comparing.

2. The method of claim 1, wherein said physicochemical properties are selected from the group consisting of Harary H index, topological polar surface area using N, O polar contributions, leverage-weighted autocorrelation of lag 0/weighted by atomic polarizabilities, R autocorrelation of lag 6/weighted by atomic polarizabilities, topological polar surface area using N, O, S, P polar contributions, Radial Distribution Function—11.0/weighted by atomic masses, valence connectivity index chi-2, phenol/enol/carboxyl OH, R autocorrelation of lag 2/weighted by atomic Sanderson electronegativities, R maximal autocorrelation of lag 4/weighted by atomic masses, graph vertex complexity index, Geary autocorrelation—lag 1/weighted by atomic masses, Ha attached to C3(sp3)/C2(sp2)/C3(sp2)/C3(sp), molecular path count of order 04, leverage-weighted autocorrelation of lag 2/unweighted, hydrophilic factor, 2st component symmetry directional WHIM index/weighted by atomic van der Waals volumes, and R maximal index/weighted by atomic polarizabilities.

3. The method of claim 2, wherein said method comprises comparing all of said physiochemical properties.

4. The method of claim 1, wherein said amino acid property descriptors are selected from the group consisting of volume, composition and polarity.

5. The method of claim 4, wherein said amino acid property descriptors are compared at specific positions in an alignment of known odorant receptor sequences.

6. The method of claim 1, wherein said method is performed in silico.

7. The method of claim 1, further comprising the step of displaying the results of said comparing on a computer screen.

8. The method of claim 1, further comprising the step of screening odorants identified in said method using an vitro assay.

9. A system for identifying an odorant ligand for an odorant receptor, comprising:
a computer program and computer software configured for comparing three or more amino acid property descriptors of an odorant receptor with three or more physicochemical properties of a plurality of odorants; and identifying one or more odorant receptor for said odorant receptor based on said comparing.

10. The system of claim 9, wherein said physicochemical properties are selected from the group consisting of Harary H index, topological polar surface area using N, O polar contributions, leverage-weighted autocorrelation of lag 0/weighted by atomic polarizabilities, R autocorrelation of lag 6/weighted by atomic polarizabilities, topological polar surface area using N, O, S, P polar contributions, Radial Distribution Function—11.0/weighted by atomic masses, valence connectivity index chi-2, phenol/enol/carboxyl OH, R autocorrelation of lag 2/weighted by atomic Sanderson electronegativities, R maximal autocorrelation of lag 4/weighted by atomic masses, graph vertex complexity index, Geary autocorrelation—lag 1/weighted by atomic masses, Ha attached to C3(sp3)/C2(sp2)/C3(sp2)/C3(sp), molecular path count of order 04, leverage-weighted autocorrelation of lag 2/unweighted, hydrophilic factor, 2st component symmetry directional WHIM index/weighted by atomic van der Waals volumes, and R maximal index/weighted by atomic polarizabilities.

11. The system of claim 10, wherein said system comprises comparing all of said physiochemical properties.

12. The system of claim 9, wherein said amino acid property descriptors are selected from the group consisting of volume, composition and polarity.

13. The system of claim 12, wherein said amino acid property descriptors are compared at specific positions in an alignment of known odorant receptor sequences.

14. The system of claim 9, wherein said computer program and computer processor are further configured for displaying the results of said comparing on a computer screen.

15. The system of claim 9, wherein said system is accessed by users within a web-based platform via a web browser across the Internet.

16. The system of claim 9, wherein said system is provided as a software package and used as a stand-alone system on a single computer.

17. A method of identifying an odorant receptor for an odorant, comprising:
a) comparing three or more amino acid property descriptors of a plurality of odorant receptors with three or more physicochemical properties of an odorant; and
b) identifying one or more odorants receptors for said odorant based on said comparing.

18. A system for identifying an odorant receptor for an odorant, comprising:
a computer program and computer software configured for three or more amino acid property descriptors of a plurality of odorant receptors with three or more physicochemical properties of an odorant; and identifying one or more odorants receptors for said odorant based on said comparing.
